(19) United States
(12) Patent Application Publication
(10) Pub. No.: US 2026/0335468 A1
(43) Pub. Date: Oct. 29, 2026

(54) MOLECULAR RADIOAFTERGLOW PROBES FOR CANCER RADIODYNAMIC THERANOSTICS

(71) Applicant: NANYANG TECHNOLOGICAL UNIVERSITY, Singapore (SG)

(72) Inventors: Kanyi PU, Singapore (SG); Jingsheng HUANG, Singapore (SG)

(21) Appl. No.: 19/485,607

(22) PCT Filed: May 20, 2024
(86) PCT No.: PCT/SG2024/050333

§ 371 Date: Nov. 18, 2025

(30) Foreign Application Priority Data
May 19, 2023 (SG) 10202301413P

(51) Int. Cl. A61K 49/00; A61B 5/00; A61K 49/04; A61P 35/00

(52) U.S. Cl. CPC A61K 49/0021; A61K 49/0056; A61K 49/0438; A61P 35/00; A61B 5/0071

(57) ABSTRACT

Disclosed herein is a compound of formula Ia or Ib:

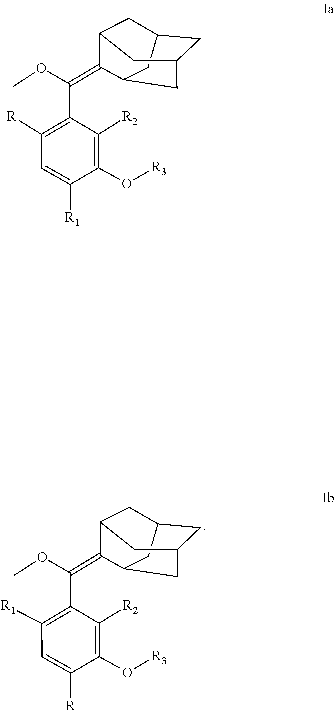

Also disclosed herein are a method for detection of cancer in vivo, the method comprising the following steps (ai) administering a compound of formula Ia or Ib, or pharmaceutically acceptable salt or solvate thereof, as aforementioned to a subject, and (aii) detecting any chemiluminescence, wherein the presence of cancer in vivo is indicated by chemiluminescence, and a method of treating cancer, the method comprising the following steps (bi) administering a compound of formula Ia or Ib, or pharmaceutically acceptable salt or solvate thereof, as aforementioned to a subject, and (bii) subjecting the subject to X-ray irradiation.

### FIELD OF INVENTION

The present disclosure generally relates to probes for cancer radiodynamic theranostics, and more particularly relates to molecular radio afterglow probes for cancer radiodynamic theranostics.

### BACKGROUND

The listing or discussion of a prior-published document in this specification should not necessarily be taken as an acknowledgement that the document is part of the state of the art or is common general knowledge.

Optical imaging agents have been pervasively applied in biology and medicine, providing molecular specificity for diagnosis and phototherapy. However, light-tissue interactions limit the imaging and therapeutic abilities of optical agents to superficial or endoscopically accessible tissues. Therefore, afterglow agents that can store photoenergy and release photons after the cessation of light irradiation have emerged to mitigate this issue. Due to the elimination of tissue autofluorescence, such photoafterglow agents can detect biomarkers in the deep tissue with high sensitivity. However, the prerequisite of light pre-irradiation causes difficulty in the repetitive induction of signal and phototherapeutic action of photoafterglow agents in deep tissues due to the limited penetration depth of light, constraining their translation potential.

In contrast to light, X-rays have deep-tissue penetration, permitting the delivery of high-energy photons into the human body for diagnostic imaging and cancer radiotherapy. Inspired by the radio-luminescence mechanism, X-rays have been lately exploited to replace light to induce afterglow imaging. However, such radio afterglow imaging (RAI) agents have been rarely reported and limited to a few rare-earth-doped inorganic nanophosphors. Similarly, X-rays can serve as the energy source to initiate the photodynamic process of optical agents to enable radiodynamic therapy (RDT). Due to the localized production of cytotoxic reactive oxygen species (ROS) from optical agents, RDT can minimize the radiation damage to normal tissues and exert tumour ablation in deep tissues. As most photosensitizers do not directly respond to X-rays, inorganic nanophosphors are often used as transducers to down-convert X-ray photons into light for the in situ excitation of photosensitizers.

Despite the translational potential of RAI and RDT in oncology, only inorganic-based nanoparticles are available; additionally, because their radio functions are biomarker independent and always switched on, cancer detection and treatment efficacies heavily rely on the concentration difference between tumour and normal tissues, leading to poor specificity. In contrast to inorganic nanoparticles, organic molecules have well-defined structures that facilitate the precision control of physiological properties. However, organic molecules often contain light atoms (such as hydrogen, carbon and oxygen) and weak spin-orbit coupling; thus, they have weak X-ray absorption, short radio-luminescence lifetime and poor singlet oxygen generation on X-ray irradiation. Consequently, no organic molecules have been reported to emit afterglow after X-ray irradiation.

Therefore, there exists a need for new molecular radio afterglow probes for cancer radiodynamic theranostics.

### SUMMARY OF INVENTION

Aspects and embodiments of the invention are provided in the following numbered clauses.
1. A compound of formula Ia or Ib:

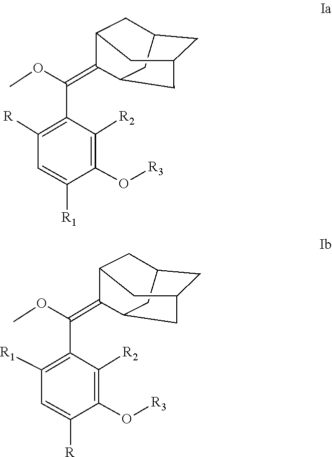

where:
R represents:

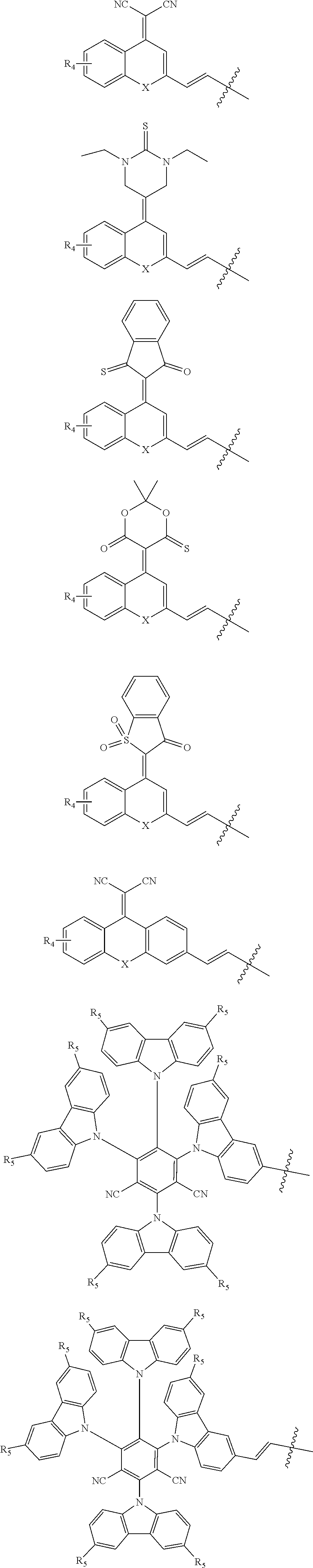

where the wiggly line represents the point of attachment to the rest of the molecule;
X represents NMe, NEt, CMe₂, SiMe₂, O, S or Se;
each R₄ is independently selected from:
   H;
   halo;

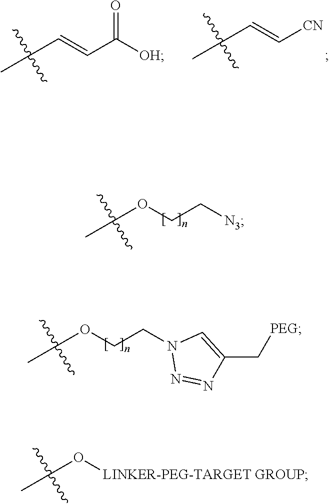

an aromatic or heteroaromatic group that is unsubstituted or substituted by CO₂H or OH,
where:
   the wiggly line represents the point of attachment to the rest of the molecule;
   PEG is PEG1K, PEG2K, or PEG5K;
   n is 0 to 11;
LINKER represents:

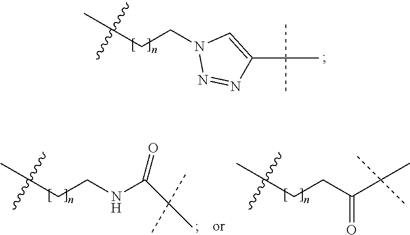

where n is 0 to 11 and the wiggly line represents the point of attachment to the oxygen atom and the dotted line represents the point of attachment to the TARGET GROUP;
TARGET GROUP represents:

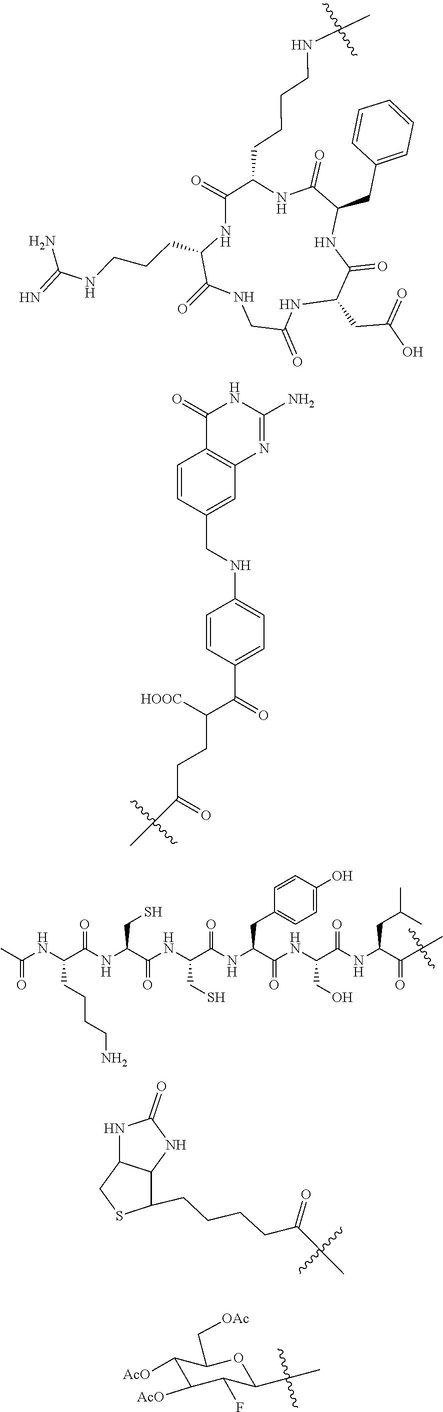

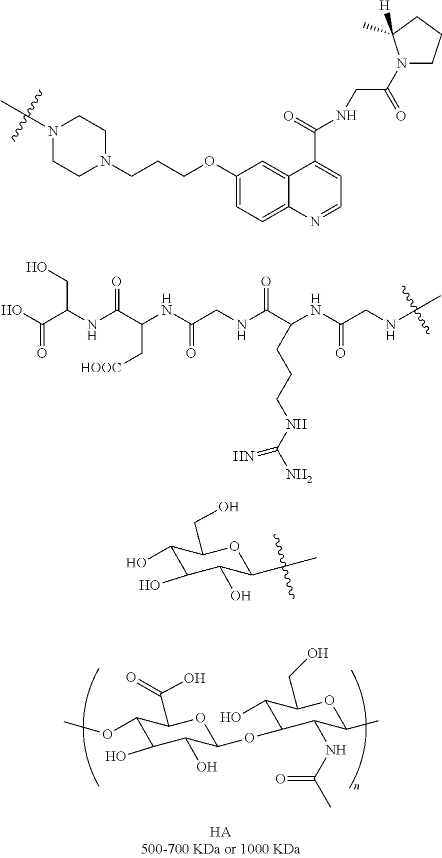

where the wiggly line represents the point of attachment to the rest of the molecule;
   each Rₛ independently represents H or halo;
   each R₁ represents H, Br or I;
   each R₂ represents H, Cl, Br, or I;
R₃ either represents:

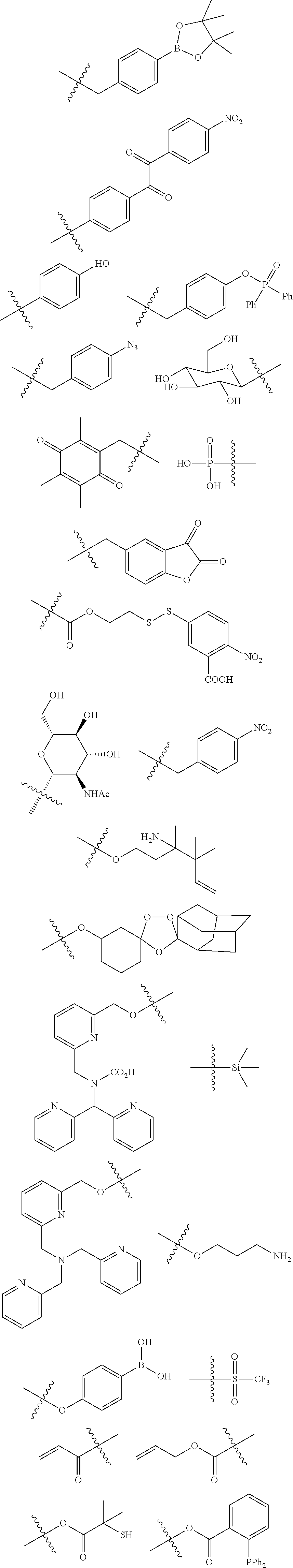

the wavy line represents the point of attachment to the rest of the molecule;
or R₃ represents -SELF INIMOLATIVE LINKER-PEPTIDE, where the self immolative linker is:

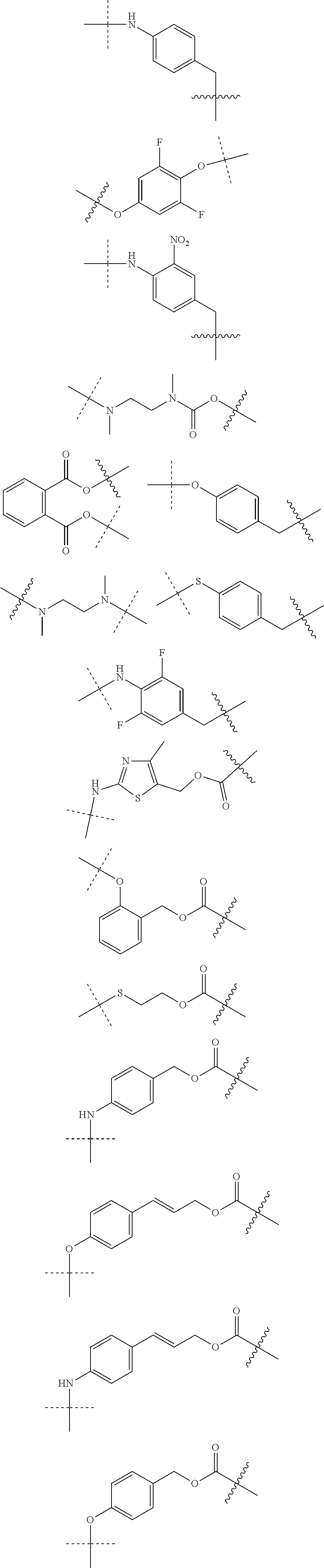

where the dotted line represents the point of attachment to the peptide and the wavy line represents the point of attachment to the rest of the molecule;
and the peptide is selected from:

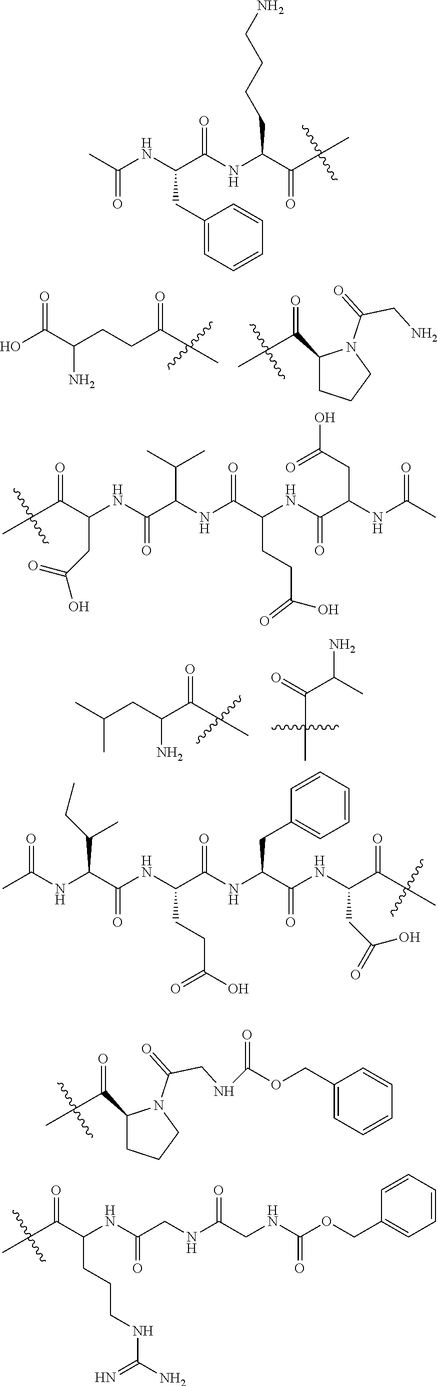

where the wavy line represents the point of attachment to the rest of the molecule,
or a pharmaceutically acceptable salt or solvate thereof.
2. The compound, or pharmaceutically acceptable salt or solvate thereof, according to Clause 1, wherein the compound has formula Ia.
3. The compound, or pharmaceutically acceptable salt or solvate thereof, according to Clause 1 or Clause 2, wherein R represents:

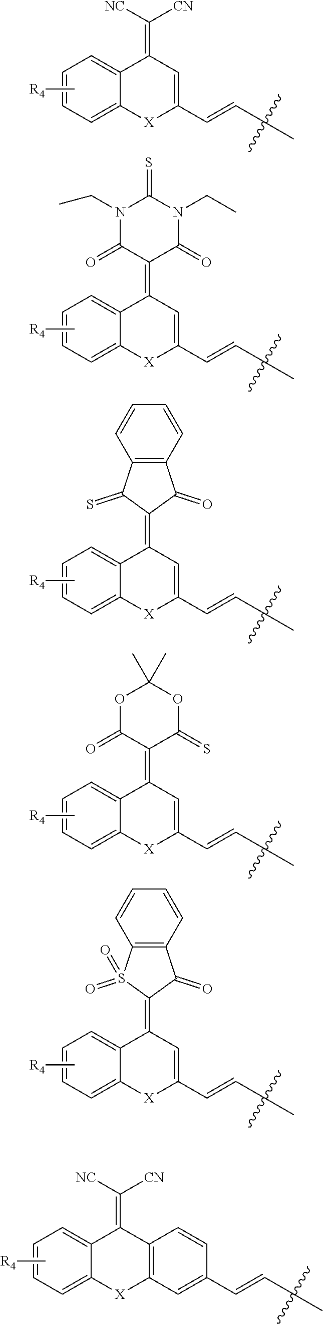

where the wiggly line represents the point of attachment to the rest of the molecule; and
X represents NEt, O, S or Se, such as 0, S or Se, such as S.
4. The compound, or pharmaceutically acceptable salt or solvate thereof, according to Clause 3, wherein R represents:

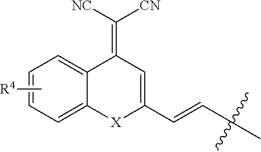

where the wiggly line represents the point of attachment to the rest of the molecule; and
X represents 0, S or Se, such as S.
5. The compound, or pharmaceutically acceptable salt or solvate thereof, according to any one of the preceding clauses, wherein R₄ is independently selected from:

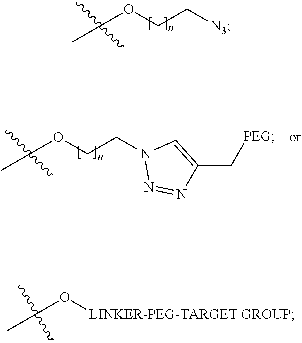

where:
   the wiggly line represents the point of attachment to the rest of the molecule;
   PEG is PEG1K, PEG2K, or PEG5K; and
   n is 0 to 11.
6. The compound, or pharmaceutically acceptable salt or solvate thereof, according to Clause 5, wherein R₄ is independently selected from:

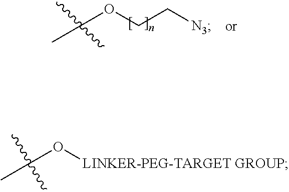

where:
   the wiggly line represents the point of attachment to the rest of the molecule;
   PEG is PEG1K, PEG2K, or PEG5K; and
   n is 3 to 5, such as 4.
7. The compound, or pharmaceutically acceptable salt or solvate thereof, according to any one of the preceding clauses, wherein, when present, LINKER represents:

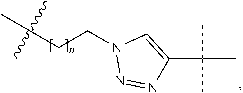

where n is 3 to 5, such as 4, and the wiggly line represents the point of attachment to the oxygen atom and the dotted line represents the point of attachment to the TARGET GROUP.
8. The compound, or pharmaceutically acceptable salt or solvate thereof, according to any one of the preceding clauses, wherein, when present, TARGET GROUP represents:

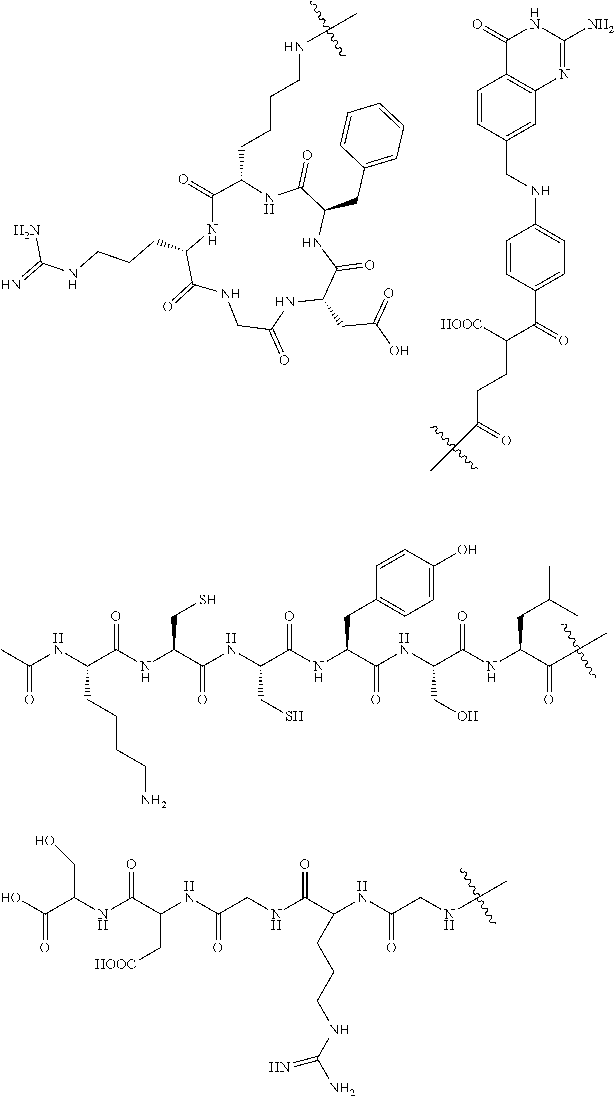 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O ; *C(=O)CCC(C(=O)O)C(=O)c1ccc(NCc2ccc3c(=O)[nH]c(N)nc3c2)cc1 ; *C(=O)[C@H](CC(C)C)NC(=O)[C@H](CO)NC(=O)[C@H](Cc1ccc(O)cc1)NC(=O)[C@H](CS)NC(=O)[C@H](CS)NC(=O)[C@H](CCCCN)NC(C)=O ; *NCC(=O)NC(CCCNC(=N)N)C(=O)NCC(=O)NC(CC(=O)O)C(=O)NC(CO)C(=O)O]

where the wiggly line represents the point of attachment to the rest of the molecule.
9. The compound, or pharmaceutically acceptable salt or solvate thereof, according to Clause 8, wherein, when present, TARGET GROUP represents:

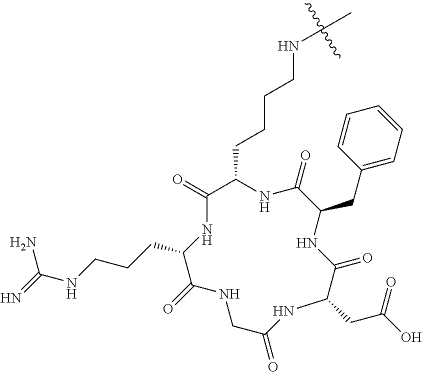 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O]

where the wiggly line represents the point of attachment to the rest of the molecule.
10. The compound, or pharmaceutically acceptable salt or solvate thereof, according to any one of the preceding clauses, wherein R₁ represents H or I.
11. The compound, or pharmaceutically acceptable salt or solvate thereof, according to any one of the preceding clauses, wherein R₂ represents H.
12. The compound, or pharmaceutically acceptable salt or solvate thereof, according to any one of the preceding clauses, wherein R₃ either represents:

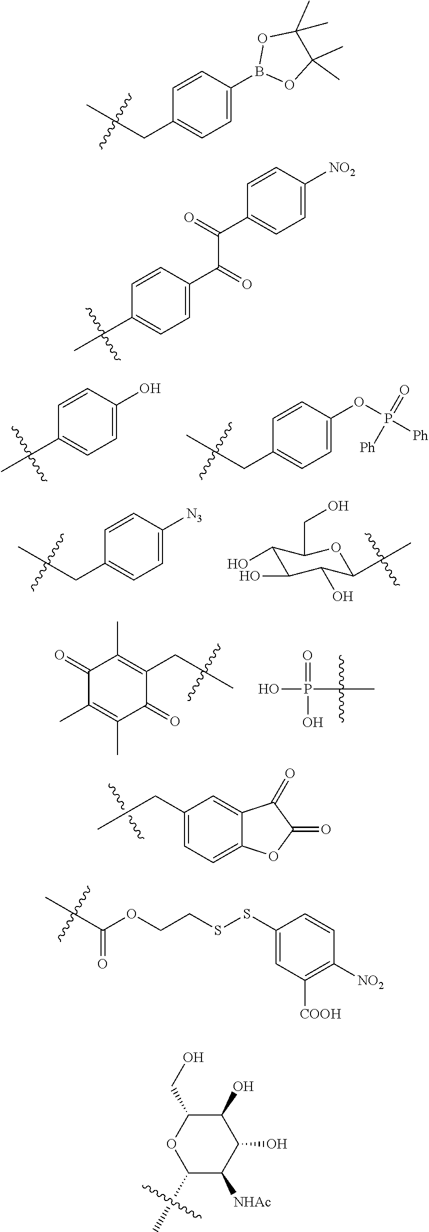

the wavy line represents the point of attachment to the rest of the molecule;
or R₃ represents -SELF INIMOLATIVE LINKER-PEPTIDE, where the self immolative linker is:

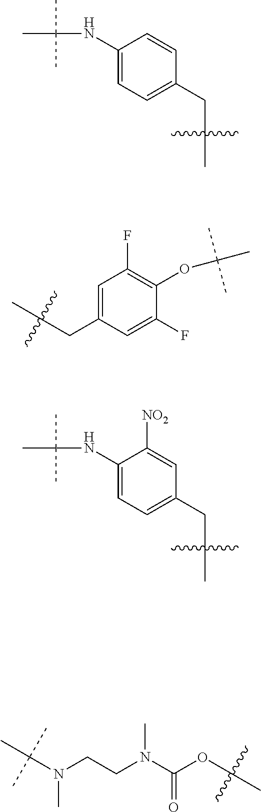

where the dotted line represents the point of attachment to the peptide and the wavy line represents the point of attachment to the rest of the molecule.
13. The compound, or pharmaceutically acceptable salt or solvate thereof, according to any one of the preceding clauses, wherein R₃ represents -SELF IMMOLATIVE LINKER-PEPTIDE, where the self immolative linker is:

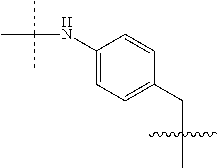

where the dotted line represents the point of attachment to the peptide and the wavy line represents the point of attachment to the rest of the molecule.
14. The compound, or pharmaceutically acceptable salt or solvate thereof, according to any one of the preceding clauses, wherein when R₃ represents -SELF IMMOLATIVE LINKER-PEPTIDE, the peptide is selected from:

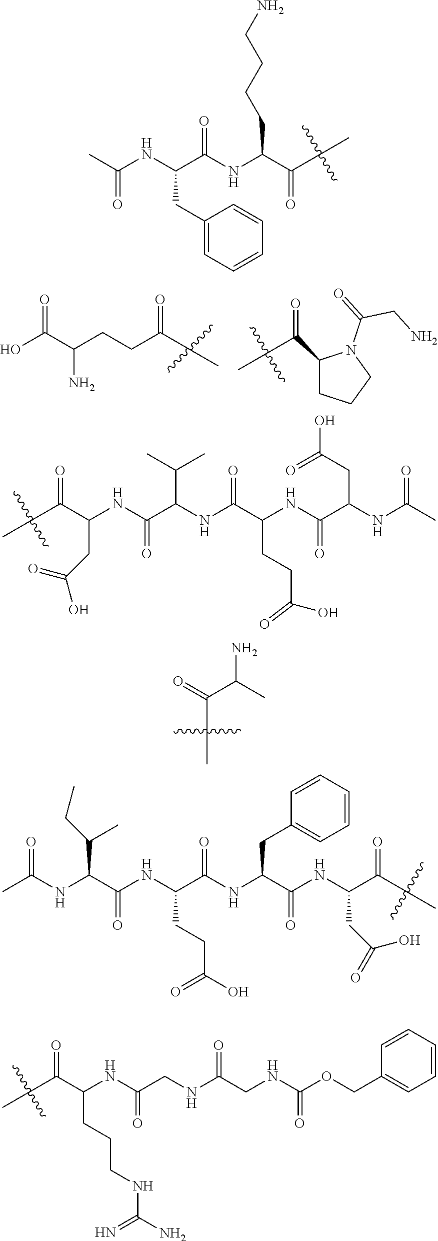

where the wavy line represents the point of attachment to the rest of the molecule.
15. The compound, or pharmaceutically acceptable salt or solvate thereof, according to Clause 14, wherein when R₃ represents -SELF IMMOLATIVE LIN4KER-PEPTIDE, the peptide is:

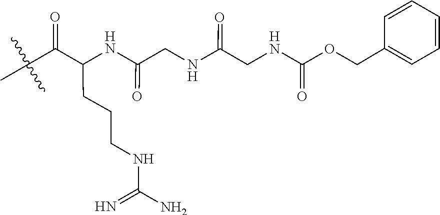 [SMILES: *C(=O)C(CCCNC(=N)N)NC(=O)CNC(=O)CNC(=O)OCc1ccccc1]

where the wavy line represents the point of attachment to the rest of the molecule.
16. The compound, or pharmaceutically acceptable salt or solvate thereof, according to any one of the preceding clauses, wherein:
   R₁ is I;
   R₂ is H;
   R is:

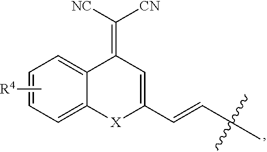

where the wavy line represents the point of attachment to the rest of the molecule;
R₄ is

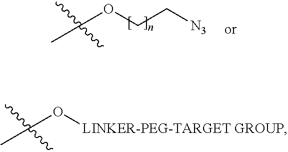

where LINKER is

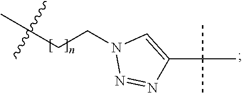

n is 4;
PEG is PEG2K;
Target Group is

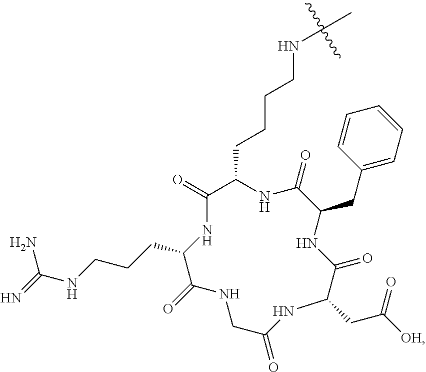 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O]

where the wiggly line represents the point of attachment to the rest of the molecule;
R₃ is -SELF IMMOLATIVE LINKER-PEPTIDE, where the self immolative linker is

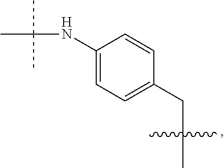

where the dotted line represents the point of attachment to the peptide and the wavy line represents the point of attachment to the rest of the molecule; and
the peptide is

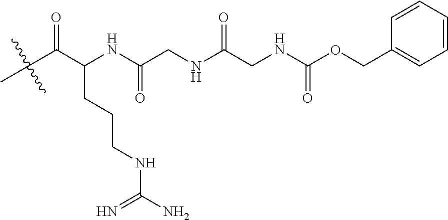 [SMILES: *C(=O)C(CCCNC(=N)N)NC(=O)CNC(=O)CNC(=O)OCc1ccccc1]

where the wavy line represents the point of attachment to the rest of the molecule.
17. A method for detection of cancer in vivo, the method comprising the following steps:
   (ai) administering a compound of formula Ia or Ib, or pharmaceutically acceptable salt or solvate thereof, according to any one of Clauses 1 to 16 to a subject; and
   (aii) detecting any chemiluminescence, wherein the presence of cancer in vivo is indicated by chemiluminescence.
18. A method of treating cancer, the method comprising the following steps:
   (bi) administering a compound of formula Ia or Ib, or pharmaceutically acceptable salt or solvate thereof, according to any one of Clauses 1 to 16 to a subject; and
   (bii) subjecting the subject to X-ray irradiation.
19. The method according to Clause 18, wherein the compound of formula Ia or formula Ib, or salt or solvate thereof, is a compound in which R₄ is

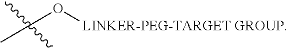

### BRIEF DESCRIPTION OF THE DRAWINGS

FIG. 1 depicts synthetic routes for radio afterglow candidates. (a) Synthesis of intermediate 4: (i) NaBH₄, methanol, r.t., 30 min, 100% yield; (ii) ICl, NaOH, methanol, 0° C., 1 h, 75%; (iii) pyridinium dichromate, pyridine, dichloromethane, r.t., 3 h, 68% yield. (b) Synthetic route for DPA_{N} and IDPA_{N}: (iv) quinoline, iodoethane, acetonitrile (ACN), reflux, overnight, 85% yield; (v) compound 5, Na, ethanol (EtOH), malononitrile, 0° C., 71% yield; (vi) for DPA_{N}: piperidine, DBQP, compound 1, ACN, reflux, N₂, 6 h, 92% yield; for IDPA_{N}: piperidine, DBQP, compound 4, ACN, reflux, N₂, 6 h, 87% yield. (c) Synthetic route for DPAₒ and IDPA_{O}. (vii) 2′-hydroxyacetophenone, ethyl acetate, NaH, THF, r.t. to reflux, overnight; then HCl, MeOH, r.t., 14 h, 65% yield; (viii) malononitrile, acetic anhydride, reflux, 2 h, 71% yield; (ix) for DPA_{O}: piperidine, DBP, compound 1, ACN, reflux, N₂, 2 h, 81%; for IDPA_{O}: piperidine, DBP, compound 4, ACN, reflux, N₂, 6 h, 67% yield. (d) Synthetic route for DPA_{Su} and IDPA_{Su}: (x) thiophenol, polyphosphoric acid, 90° C., 1 h, 68% yield; (xi) compound 6, malononitrile, acetic anhydride, reflux, 2 h, 53% yield; (xii) for DPA_{Su}: piperidine, DBTP, compound 1, ACN, reflux, N₂, 2 h, 76% yield; for IDPA_{Su}: piperidine, DBTP, compound 4, ACN, reflux, N₂, 2 h, 63% yield. (e) Synthetic route for DPA_{Se} and IDPA_{Se}: (xiii) 2-bromobenzaldehyde, propynylmagnesium bromide, THF, r.t., 1 h, 100% yield; (xiv) o-iodoxybenzoic acid, dimethylsulfoxide (DMSO), r.t., 96% yield; (xv) Se, NaBH₄, DMF, 105° C., 85% yield; (xvi) compound 10, Malononitrile, acetic anhydride, reflux, 2 h, 31% yield; (xvii) for DPA_{Se}: piperidine, DBSeP, compound 1, ACN, reflux, N₂, 2 h, 52%; for IDPA_{Se}: piperidine, DBSeP, compound 4, ACN, reflux, N₂, 2 h, 38% yield.

FIG. 2 depicts the normalized ultraviolet (UV) spectra of radio afterglow candidates (a,b) before and (c,d) after Xray irradiation at 250 μGy·s⁻¹ for 20 s in phosphate buffered saline (PBS, 0.01 M, pH 7.4, 50% DMSO, V/V) solution.

FIG. 3 depicts the normalized fluorescence spectra of radio afterglow candidates (a,b) before and (c,d) after X-ray irradiation at 250 μGy·s⁻¹ for 20 s in PBS (0.01 M, pH 7.4, 50% DMSO, V/V) solution at 450 nm excitation.

FIG. 4 depicts synthesis and characterization of radio afterglow dynamic luminophores. (a) Chemical structures of DPAs and IDPAs. (b) Schematic of the proposed radio afterglow mechanism. X-ray photons first interact with atoms in IDPA_{X} through the photoelectric effect and Compton scattering, ejecting high-energy electrons (ionization). These hot electrons further interact with the surrounding atoms, eliciting a cascade of secondary electrons with decreased energy and formation of electron-hole pairs (thermalization). Owing to the presence of iodine and sulfur, a large number of electrons are populated as triplet excitons. These excitons jump back to the ground state, transferring energy to the surrounding oxygen molecules and generating ¹O₂ (sensitization). The in situ-generated ¹O₂ reacts with IDPA_{X} via a cycloaddition reaction and converts it into a dioxetane intermediate. At last, this intermediate gradually decomposes into the corresponding activated products (activated IDPA_{X}) and releases photons (afterglow). (c-e) Radio afterglow spectra (c), intensities (d) and half-lives (e) of DPAs and IDPAs (100 μM) in PBS (0.01 M, pH 7.4, 50% DMSO) after X-ray irradiation (5 mGy, 250 μGy s⁻¹ for 20 s) (n=3 independent experiments). (f) Radio afterglow intensities of IDPA_{Su} after ten cycles of X-ray irradiation. X-ray was ‘on’ at 250 μGy s⁻¹ for 20 s and ‘off’ for 12 h in each cycle (n=3 independent experiments). (g) High-performance liquid chromatography (HPLC) analysis of IDPA_{Su} before and after X-ray irradiation (2 Gy, 100 mGy s⁻¹ for 20 s). (h) Electron spin resonance (ESR) spectra showing ¹O₂ generation from IDPA_{Su} (100 PM) in PBS (0.01 M, pH 7.4, 50% DMSO) before and after X-ray irradiation (5 mGy, 250 ρGy s⁻¹ for 20 s) by 2,2,6,6-tetramethylpiperidine (TEMP) as the trapping agent and NaN₃ (1 mM) as the ¹O₂ quencher. (i) ¹O₂ generation from DPAs, IDPAs (100 μM) and commercial radio sensitizers (acridine orange (AO), hematoporphyrin (HP) and verteporfin (VP)) in PBS (0.01 M, pH 7.4, 50% DMSO) after X-ray irradiation at 250 μGy s⁻¹ for 20 s (n=3 independent experiments). Data were presented as mean±s.d.

FIG. 5 depicts (a) decay of radio afterglow from candidates (100 M) in PBS (0.01 M, pH 7.4, 50% DMSO) solution after X-ray irradiation at 250 μGy·s⁻¹ for 20 s. (b) Representative images of radio afterglow from IDPASu solution at different timepoints (10, 120, 360, 720, 1200 and 1500 min) after X-ray irradiation at 250 μGy·s⁻¹ for 20 s.

FIG. 6 depicts HPLC analysis of IDPA_{Su} after one and 10 on-off cycles of radio afterglow (each cycle at 250 μGy·s⁻¹ for 20 s).

FIG. 7 depicts radio afterglow mechanism and photophysical property study. (a) Proposed radio afterglow mechanism of IDPA_{Su} and HRMS analysis of the new peaks at 6.0 (b) and 14.8 min (c) in FIG. 4g, confirming the generation of dioxetane intermediate and activated IDPA_{Su}. (d) Radio afterglow intensity of IDPA_{Su} as a function of its concentration in PBS (0.01 M, pH 7.4, 50% DMSO) after X-ray irradiation at 250 μGy·s⁻¹ for 20 s. Data were presented as mean±s.d (n=3 independent experiments). (e) Temperature of PBS and IDPA_{Su} (100 mM) in PBS (0.01 M, pH 7.4, 50% DMSO) before and after X-ray irradiation at 250 μGy·s⁻¹ for 20 s at room temperature. Data were presented as mean±s.d (n=3 independent experiments).

FIG. 8 depicts (a) chemical structure of DIDPA_{O} and DIDPA_{S}. Radio afterglow intensity of DIDPA_{O} (b) and DIDPA_{S} (c) (100 mM) after X-ray irradiation at different dosages (0, 5, 50 and 500 mGy) in PBS (0.01 M, pH 7.4, 50% DMSO, V/V) solution. ESR spectra for ¹O₂ generation from DIDPA_{O} (d) and DIDPA_{S} (e) with and without X-ray irradiation (5 mGy). (f) Quantification of ESR signal from (d) and (e).

FIG. 9 depicts standard curve relationship between the ESR signal and different concentrations of ¹O₂ when incubated with TEMP (10 mM). Data were presented as mean±s.d., n=3 independent experiments. ¹O₂ was quantitatively generated by addition of NaOCl to H₂O₂.

FIG. 10 depicts detection of O₂·⁻ and ·OH generated from IDPA_{Su} solution upon X-ray irradiation. (a) Mechanism of DHR123 probe for O₂·⁻ detection. (b) Fluorescence signals of DHR123 as a function of concentrations of O₂·⁻ at λₑₓ 505 nm and λₑₘ 534 nm. Data were presented as mean±s.d., n=3 independent experiments. (c) Fluorescence signals of DHR123 in the presence of IDPA_{Su}(10 M) upon X-ray irradiation of 0, 5 mGy (250 μGy·s⁻¹ for 20 s), and 2 Gy (100 mGy·s⁻¹ for 20 s) in PBS (0.01 M, pH 7.4, containing 50% DMSO) solution. Statistical significance was calculated via one-way ANOVA with a Tukey post-hoc test, DHR123 (+X-ray, 250 μGy·s⁻¹ for 20 s or 100 mGy·s⁻¹ for 20 s) versus DHR123 (−X-ray), ns: not significant while DHR123+IDPA_{Su} (+X-ray, 100 mGy·s⁻¹) versus DHR123+IDPA_{Su} (−X-ray), ****p=2×10⁻⁶. Data were presented as mean±s.d., n=3 independent experiments. (d) Mechanism of HPF probe for ·OH detection. (e) Fluorescence signals of HPF as a function of the concentrations of ·OH at λₑₓ 450 nm and λₑₘ 515 nm. Data were presented as mean±s.d., n=3 independent experiments. (f) Fluorescence signals of DHR123 in the presence of IDPA_{Su} upon different dosages of X-ray irradiation in PBS (0.01 M, pH 7.4, containing 50% DMF) solution. Data were presented as mean±s.d (n=3 independent experiments). Statistical significance was calculated via one-way ANOVA with a Tukey post-hoc test; HPF (+X-ray, 250 μGy·s⁻¹ for 20 s or 100 mGy·s⁻¹ for 20 s) versus HPF (−X-ray): ns: not significant; HPF+IDPA_{Su}(+X-ray, 250 μGy·s⁻¹ for 20 s or 100 mGy·s⁻¹ for 20 s) versus HPF+IDPA_{Su}(−X-ray): ns: not significant.

FIG. 11 depicts deep-tissue radio afterglow dynamic process. (a) Schematic of afterglow induction through chicken breast tissue by X-ray or light irradiation and detection without chicken breast tissue. The X-ray source and xenon lamp were optimized to ensure that all the samples at 0 cm (without coverage) received the same dose of X-rays (5 mGy, 250 μGy·s⁻¹ for 20 s) or light (180 mW cm⁻² for 20 s) and then the afterglow signal was recorded without chicken breast tissues. (b) Schematic of the advantages of radio afterglow over photoafterglow. (c) ¹O₂ generation from IDPA_{Su} (100 μM) in PBS (0.01 M, pH 7.4, 50% DMSO) after X-ray or light irradiation through chicken breast tissues (n=3 independent experiments). (d) Afterglow images of IDPA_{Su} (100 M) in PBS (0.01 M, pH 7.4, 50% DMSO) after X-ray or light irradiation at different tissue depths. (e) Signals of radio afterglow and photoafterglow at different tissue depths (scale breaks from 0.01 to 0.02) (n=3 independent experiments). Data are presented as mean±s.d.

FIG. 12 depicts (a,b) cell viabilities of U87 cells incubated with IDPA_{Su} at different concentrations (0, 2.5, 5, 10, 25, 50, and 100 μM) for 24 h without (a) and with X-ray irradiation through chicken breast tissue of different thicknesses (0, 5, 8 and 15 cm) (b). All data were presented as mean±s.d., n=5 independent experiments.

FIG. 13 depicts (a) representative radio afterglow images of tumor-free Balb/c nude mice subcutaneously injected with 20 μl of IDPA_{Su} solution at different concentrations (100, 50, 20, 10, 5, 1, 0.5, 0 M). (b) Radio afterglow intensities as a function of IDPA_{Su} concentrations in (a). Mice were irradiated with Xray at 5 mGy (250 μGy·s⁻¹ for 20 s) prior to signal detection with the acquisition time of 60 s. Data were presented as mean±s.d (n=3 independent mice).

FIG. 14 depicts in vivo radio afterglow imaging and radiodynamic therapy after intratumoral injection of IDPA_{Su}. (a) Schematic illustration of IDPA_{Su}-mediated cancer theranostics in subcutaneous U87 tumor-bearing mice. (b) In vivo radio afterglow spectra on U87 tumors of nude mice at 0.5 h post-injection of IDPA_{Su} (0.1 μmol·kg⁻¹) with and without X-ray irradiation at 5 mGy (250 μGy·s⁻¹ for 20 s) with each emission with automatic exposure time. (c,d) Representative radio afterglow images (c) and radio afterglow intensities (d) on tumors of living nude mice at 0, 0.5, 1, 4, 6, 8, 12, 16, or 24 h post-injection of IDPA_{Su}(0.1 μmol·kg⁻¹) in PBS (0.01 M, pH 7.4). IDPA_{Su} was solubilized by (2-carboxyethyl)-o-cyclodextrin sodium salt (β-CD, 0.1 μmol·kg⁻¹). At each timepoint, mice were irradiated with X-ray at 5 mGy (250 μGy·s⁻¹ for 20 s) followed by signal detection with the acquisition time of 60 s. Data were presented as mean±s.d., n=3 independent mice. (e) Confocal fluorescence images of tumor tissues from PBS and IDPA_{Su}-injected (0.1 μmol·kg⁻¹) U87-tumor-bearing mice with or without X-ray irradiation (0.1 Gy, 5 mGy·s⁻¹ for 20 s). Blue fluorescence signals from cell nucleus stained with DAPI, and green fluorescence signals from tumor tissues stained with Singlet Oxygen Sensor Green (SOSG). (f) Quantification of SOSG signals in tumor tissues (e) (n=3 mice). (g,h) Tumor growth (g) and body weight (h) curves of U87 tumor-bearing mice after indicated treatments (n=5 mice). Statistical significance in (g) was calculated by two-tailed Student's t-test; PBS+X-ray versus PBS group: ns: not significant while IDPA_{Su}+X-ray versus PBS group ****p=3.7×10⁻⁵. (i, j) Confocal fluorescence images of TUNEL-stained tumor tissues after indicated treatments. Nuclei were stained with DAPI (blue), and DNA fragments were stained with TUNEL (green) (n =3 independent experiments). (j) Representative images of haematoxylin and eosin (H&E)-stained tumor tissues after indicated treatments (n=3 independent experiments).

FIG. 15 depicts in vitro studies for radio afterglow dynamic cancer theranostics. (a) Schematic of the sensing mechanism of MRAP for cathepsin B (CatB). (b) Radio afterglow spectra of molecular radio afterglow dynamic probe (MRAP, 100 μM) in the presence and absence of CatB (5 μg ml⁻¹) in PBS (0.01 M, pH 7.4), EDTA (1 mM) and glutathione (5 mM) for 6 h incubation. X-ray irradiation: 5 mGy, 250 μGy s⁻¹ for 20 s. (c) Radio afterglow signals of MRAP (100 μM) after 6 h incubation with different enzymes (ALP, APN, furin, GGT, NTR, (β-gal, uPA and CatB). X-ray irradiation: 5 mGy, 250 μGy s⁻¹ for 20 s (n=3 independent experiments). (d) ESR spectra for ¹O₂ generation from MRAP before and after the cleavage of CatB with and without X-ray irradiation (2 Gy, 100 mGy s⁻¹ for 20 s). (e) Schematic of activatable RAI and RDT of MRAP for CatB-overexpressed U87 cells. (f) Radio afterglow intensities of U87 and 3T3 cells at different numbers (1, 2 and 4×10⁴ per well) after incubation with MRAP (100 μM) for 10 h. X-ray irradiation: 5 mGy, 250 μGy s⁻¹ for 20 s. The control group (U87+CA-074): U87 cells were preincubated with CA-074 (1 mM) for 10 h (n=4 independent experiments). The inset shows the representative radio afterglow images of U87 and 3T3 cells after the indicated treatments. (g) Viabilities of CatBnegative cells (3T3, RAW 264.7, THP-1 and 293-T) and CatB-positive cells (U87, 4T1, B16-F10 and A549) after incubation with MRAP at different concentrations for 24 h. X-ray irradiation: 2 Gy, 100 mGy s⁻¹ for 20 s (n=5 independent experiments). Data are presented as mean±s.d.

FIG. 16 depicts (a) synthesis of uncaged MRAP: (i) ethyl acetoacetate, 4-methoxybenzenethiol, polyphosphate, 90° C., 30 min, 68% yield; (ii) compound 11, BBr₃, dichloromethane (DCM), 0° C., 6 h, 95% yield; (iii) compound 12, K₂CO₃, 1-azido-4-iodobutane, DMF, 80° C., overnight, 87% yield; (iv) compound 13, malononitrile, AcOAc, 140° C., 60 min, 45% yield; (v) compound 14, piperidine, compound 4, ACN, reflux, N₂, 2 h, 92% yield; (vi) compound 15, CuSO₄·5H₂O, ascorbic acid sodium, DMSO/H₂O (V/V, 9/1), alkyne-PEG2000-cRGD, 95% yield. (b) Synthetic route for activatable MRAP and cRGD-free MRAP: (vii) NaHCO₃, dimethoxyethane, 16 h, from 0° C. to r.t., 96% yield, (viii) N-ethoxycarbonyl-2-ethoxy-1,2-dihydroquinoline, THF, 8 h, r.t., 90% yield; (ix) PBr₃, THF, 0° C., without purification; (x) Cs₂CO₃, K₂CO₃, ACN, overnight, r.t., 67% yield; (xi) water-soluble moiety (alkyne-PEG2000-cRGD or alkyne-PEG20000Me), CuSO₄5H₂O, ascorbic acid sodium, DMSO/H₂O (V/V, 9/1), equivalent production (MRAP and cRGD-free MRAP).

FIG. 17 depicts (a,b) absorbance (a), fluorescence (b) spectra of MRAP (100 μM) in absence and presence of cathepsin B (CatB, 5 g/mL) in PBS (0.01 M, pH 7.4, 1 mM EDTA, 5 mM glutathione) for 6 h incubation at X-ray irradiation (5 mGy: 250 μGy·s⁻¹ for 20 s). (c) HPLC analysis of MRAP precursor compound 18 before and after cleavage by CatB. (d) Radio afterglow intensities of MRAP (100 μM) after incubation with CatB activation, followed by X-ray irradiation (n=3 independent experiments, mean±s.d.). (e) Radio afterglow intensities of MRAP as a function of CatB concentrations after X-ray irradiation (n=3 independent experiments, mean±s.d.). (f) ¹O₂ generation of MRAP (100 μM) in the presence of CatB (5 g/mL) upon X-ray irradiation through different chicken breast tissue depths (n=3 independent experiments, mean±s.d.). Two-tailed Student's t-test; thickness at 15 cm group versus -CatB group, *p=0.023.

FIG. 18 depicts (a) viabilities of U87 cells incubated with MRAP at different concentrations for 24 h (n=5 independent experiments, mean±s.d.). (b,c) Viabilities of U87 (b) and 3T3 cells (c) incubated with MRAP at different concentrations for 24 h and irradiated with X-ray through chicken breast tissues of different thicknesses (n=5 independent experiments, mean±s.d.). (d) Viability of U87 cells pre-treated with CA-074 (1 mM, CatB inhibitor) for 10 h and incubated with MRAP without covered tissue, followed by X-ray irradiation at 0 and 2 Gy (100 mGy·s⁻¹ for 20 s) (n=5 independent experiments). Data were shown as mean±s.d.

FIG. 19 depicts MRAP-mediated RAI and RDT. (a) Schematic of MRAP for RAI and RDT of orthotopic glioblastoma. (b) Representative radio afterglow images of mice at different timepoints (0, 2, 6, 12, 24, 36 and 72 h) after an intravenous injection of MRAP and cRGD-free MRAP (10 μmol kg⁻¹) with X-ray irradiation (5 mGy, 250 μGy s⁻¹ for 20 s). (c) Radio afterglow intensities of brain areas of mice in b as a function of post-injection time (n=3 independent mice). Two-tailed Student's t-test; MRAP versus MRAP (no tumour), ***p=8×10⁴. (d,e) Tumour growth (d) and survival rate (e) curves of U87-tumour-bearing mice after the indicated treatments (n=5 independent mice). The irradiation groups under X-ray irradiation at 0.8 Gy (40 mGy S⁻¹ for 20 s) for four fractions. Two-tailed Student's t-test; MRAP+X-ray versus PBS, ****p=6×10⁻⁶. (f) Representative images and tumour areas of H&E-stained brain sections of mice treated with PBS+X-ray and MRAP+X-ray on day 21. Tumour areas: PBS+X-ray (average, ~9.24 mm²), MRAP+X-ray (average, ~1.61 mm²). Data are presented as mean±s.d., n=3 independent mice. (g) Histological analysis of H&E- and immunofluorescence-stained sections of the orthotopic glioblastoma of mice treated with PBS and MRAP with or without X-ray irradiation. Nuclei stained with DALPI (blue), tissue ¹O₂ shown by SOSG (green), caspase-1 stained with anti-caspase-1 antibody (red) and DNA fragments stained with TUNEL (green) (n=3 independent experiments).

FIG. 20 depicts biodistribution and pharmacokinetics studies. (a) Quantification of cRGD-free MRAP and MRAP in main organs from orthotopic U87 tumor-bearing nude mice at 24 h after intravenous injection of the corresponding probe (10 μmol·kg⁻¹) (n=3 independent mice). (b,c) The percent injected dose (ID %) of MRAP (b) and cRGD-free MRAP (c) in blood circulation after intravenous injection (10 μmol·kg⁻¹) into healthy nude mice (n=3 independent mice). Data were shown as mean±s.d.

FIG. 21 depicts bioluminescence imaging of orthotopic U87-Luc tumor-bearing mice at day 0, 3, 6, 9, 12, 15, 18, and 21 during indicated treatments (n=5 mice).

FIG. 22 depicts (a) representative images of H&E staining of whole brain sections from PBS and MRAP groups at day 21 and an enlarged view of tumor area from MRAP+X-ray and PBS+X-ray groups (FIG. 19f). (b) Body weight of U87 tumor-bearing mice after indicated treatments (n=5 mice, mean±s.d.). (c) Histological analysis of the major organs. Histological H&E-stained main organs of mice collected staining of heart, liver, lung and kidney from intracranial U87 tumor-bearing nude mice sacrificed after 21 days of evaluation. Images are representative of three biologically independent mice.

FIG. 23 depicts MRAP-mediated ultrasmall-tumour detection and precision tumour ablation. (a) Schematic of radio afterglow and bioluminescence for ultrasmall-tumour detection. (b) Representative bioluminescence and radio afterglow images of U87-Luc-tumour-bearing mice with different tumour sizes. The mice were intraperitoneally injected with d-luciferin, sodium salt (500 μmol kg⁻¹) or intravenously injected with MRAP (10 μmol kg⁻¹) with X-ray irradiation (5 mGy, 250 μGy S⁻¹ for 20 s) before imaging. (c) Radio afterglow and bioluminescence intensities as a function of tumour sizes (n=3 independent mice). (d) Schematic, in vivo and ex vivo RAI of brain areas for biocompatibility and precise therapy studies. U87-Luc-tumour-bearing mice were locally injected with MRAP or uncaged MRAP (0.5 mol kg⁻¹) at 30 min before X-ray irradiation (2 Gy, 100 mGy S⁻¹ for 20 s) and RAI (120 s acquisition time). (e) Chemical structure of always-on uncaged MRAP. (f) Radio afterglow intensities of in vivo and ex vivo images of the brain areas in d (n=3 independent mice). (g) H&E and immunofluorescent section staining studies of the brain sections from d. Nuclei were stained with DAPI (blue) and caspase-3 was stained by anti-caspase-3 antibody (red).

FIG. 24 depicts routine blood analysis of normal Kunming mice after intravenous injections with PBS, cRGD-free MRAP (20 μmol·kg⁻¹) and MRAP (20 μmol·kg⁻¹) with a dose interval of 24 h for once and five times, respectively (n=3 independent mice). Data were shown as mean±s.d.. WBC: white blood cells (normal range: 0.8-6.8); Lymph #: lymphocyte number (normal range: 0.7-5.7); Mon #: monocyte number (normal range: 0.0-0.3); Gran #: granulocyte number (normal range: 0.1-1.8); Lymph %: lymphocyte (%) (normal range: 55.8-90.6); Mon %: monocyte (%) (normal range: 1.8-6.0); Gran %: granulocyte (%) (normal range: 8.6-38.9); RBC: red blood cells (normal range: 6.36-9.42); HGB: hemoglobin (normal range: 110-143); HCT: hematocrit (normal range: 34.6-44.6); MCV: Mean Corpuscular Volume (normal range: 48.2-58.3); MCH: Mean Corpuscular Hemoglobin (normal range: 15.8-19); MCHC: Mean Corpuscular Hemoglobin Concentration (normal range: 302-353); RDW: Red Cell Distribution Width (normal range: 13-17); PLT: Platelets (normal range: 450-1590); MPV: Mean Platelet Volume (normal range: 3.8-6.0).

FIG. 25 depicts (a) schematic illustration of radio afterglow and photoafterglow induction and detection through chicken breast tissue. (b) Signal-to-background ratio (SBR) of radio afterglow and photoafterglow of IDPA_{Su} (100 μM) in PBS (0.01 M, pH 7.4, 50% DMSO) placed under chicken breast tissue with different thickness (0, 1, 2, 4, 4.5 and 5 cm) after X-ray irradiation or white light irradiation. Data were presented as mean±s.d. (n=3 independent experiments). X-ray source and Xenon lamp were optimized to ensure that all samples at 0 cm (without coverage) received same dose of X-ray (5 mGy, 250 μGy·s⁻¹ for 20 s) or light (180 mW·cm² for 20 s) and then afterglow signal was recorded with chicken breast tissues. (c) Representative images of radio afterglow and photoafterglow of IDPA_{Su} (100 M) in PBS (0.01 M, pH 7.4, 50% DMSO) placed under chicken breast tissue with different thickness (0, 1, 2, 4, 4.5 and 5 cm) after X-ray irradiation or white light irradiation.

### DESCRIPTION OF INVENTION

It has been surprisingly found that the compounds disclosed herein are activatable near-infrared radio afterglow probes for cancer radiodynamic theranostics. These compounds allow the detection of a diminutive tumour with superb contrast and perform radiotherapy for deep-seated tumour with molecular precision at a low dosage. Further, modification of these compounds can potentially allow for image-guided surgery and for monitoring cell therapy.

Thus, in a first aspect of the invention, there is provided a compound of formula Ia or Ib:

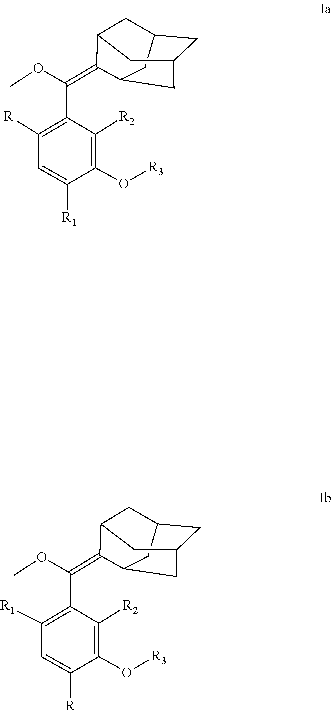

where:
R represents:

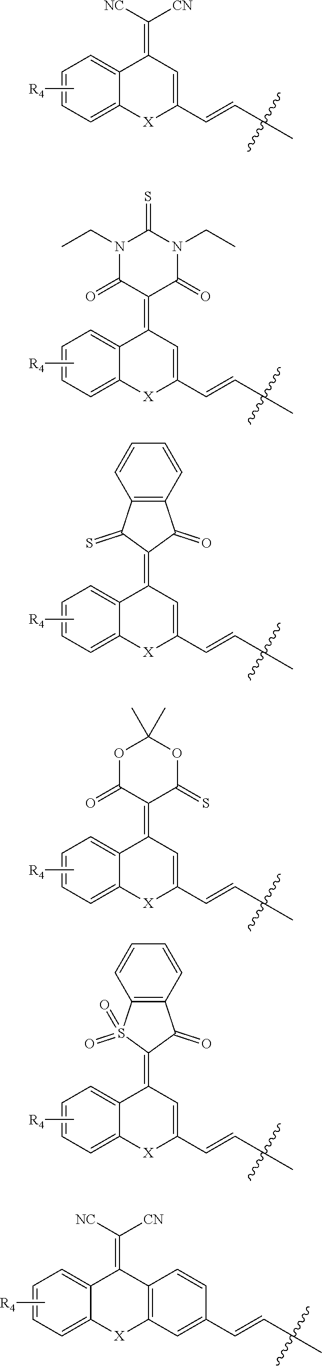

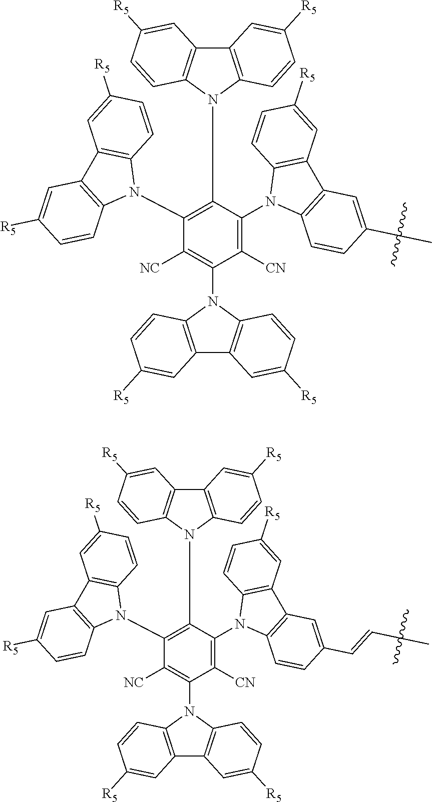

where the wiggly line represents the point of attachment to the rest of the molecule;
X represents NMe, NEt, CMe₂, SiMe₂, O, S or Se;
each R₄ is independently selected from:
   H;
   halo;

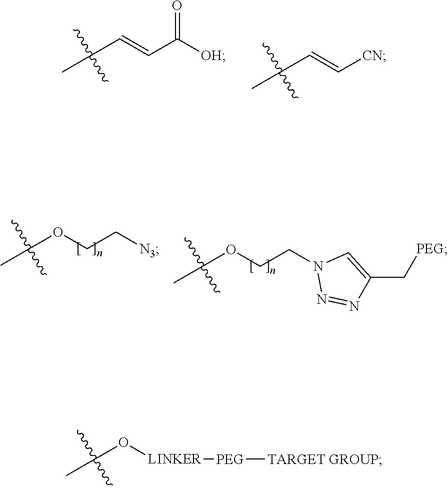

or
an aromatic or heteroaromatic group that is unsubstituted or substituted by CO₂H or OH,
where:
   the wiggly line represents the point of attachment to the rest of the molecule;
   PEG is PEG1K, PEG2K, or PEG5K;
   n is 0 to 11;
LINKER represents:

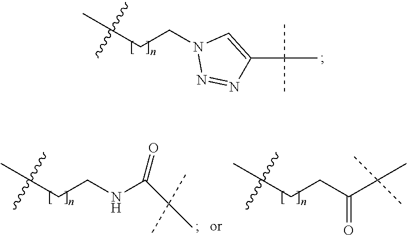

where n is 0 to 11 and the wiggly line represents the point of attachment to the oxygen atom and the dotted line represents the point of attachment to the TARGET GROUP;
TARGET GROUP represents:

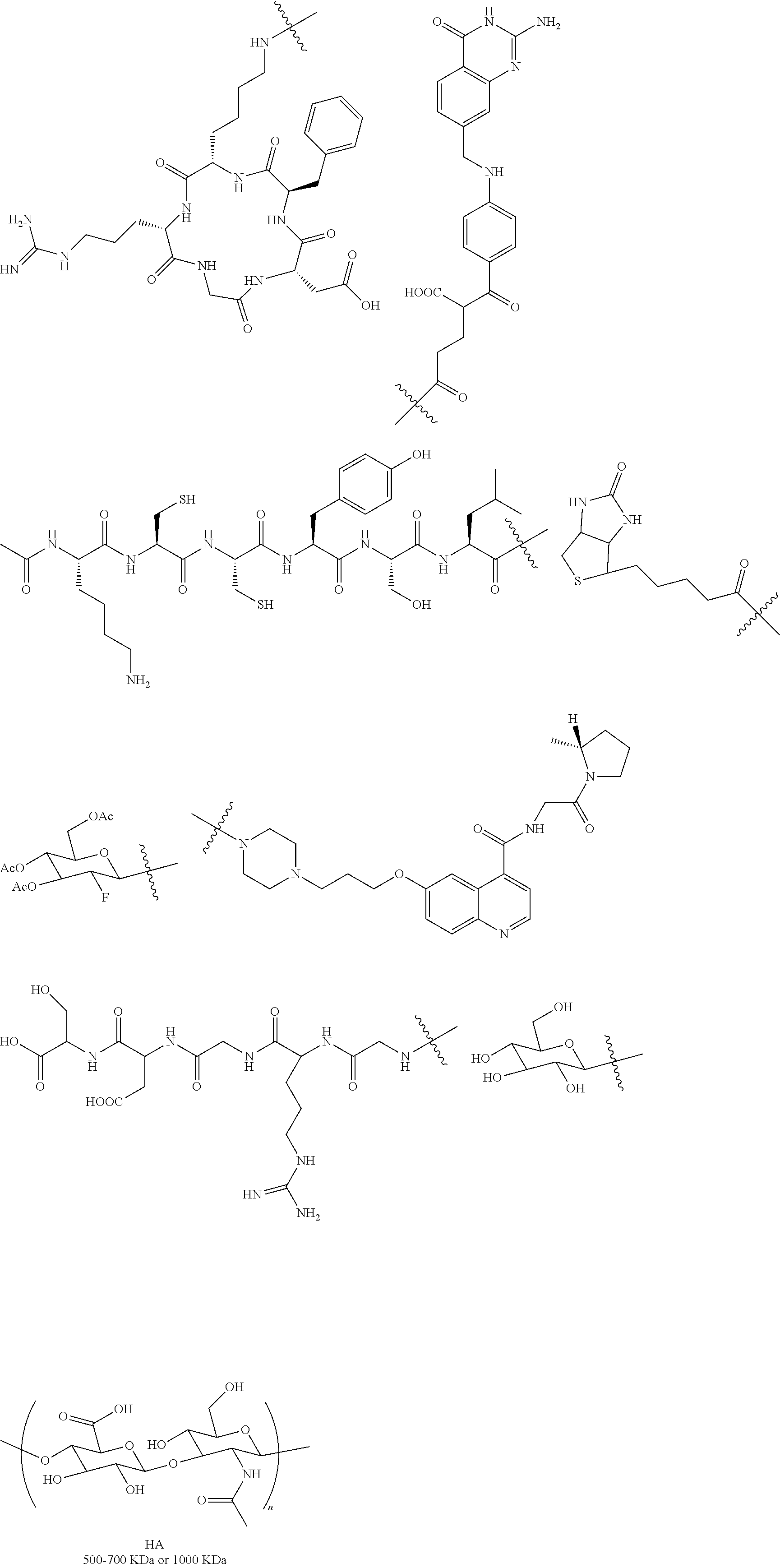

where the wiggly line represents the point of attachment to the rest of the molecule;
each Rₛ independently represents H or halo;
each R₁ represents H, Br or I;
each R₂ represents H, Cl, Br, or I;
R₃ either represents:

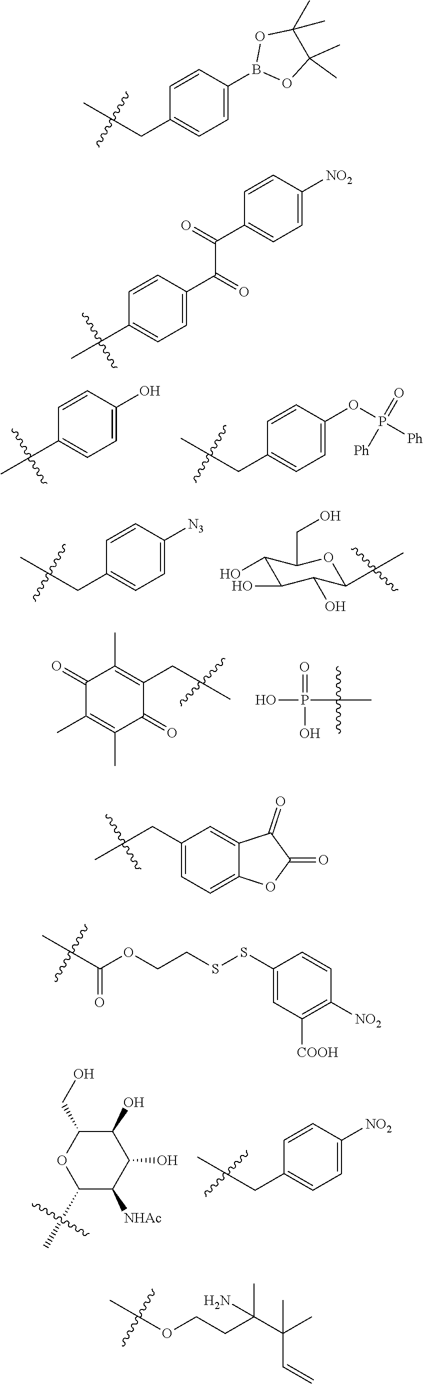

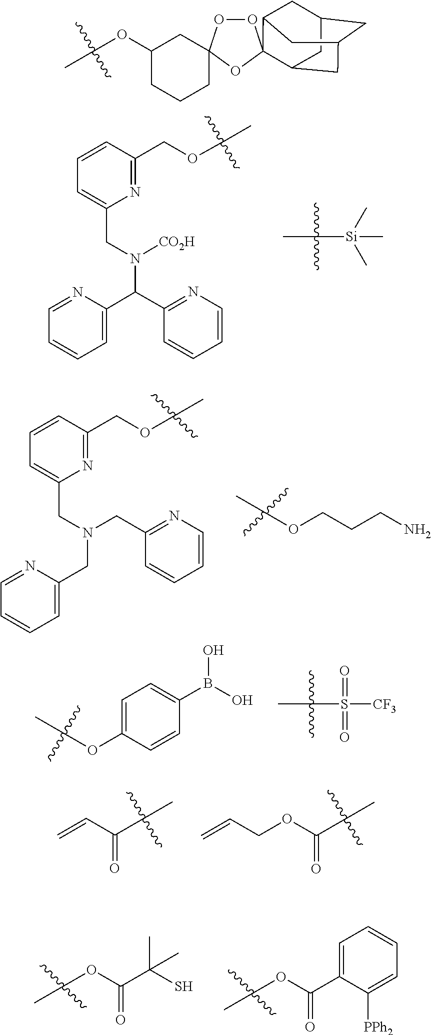

the wavy line represents the point of attachment to the rest of the molecule;
or R₃ represents -SELF IMMOLATIVE LINKER-PEPTIDE, where the self immolative linker is:

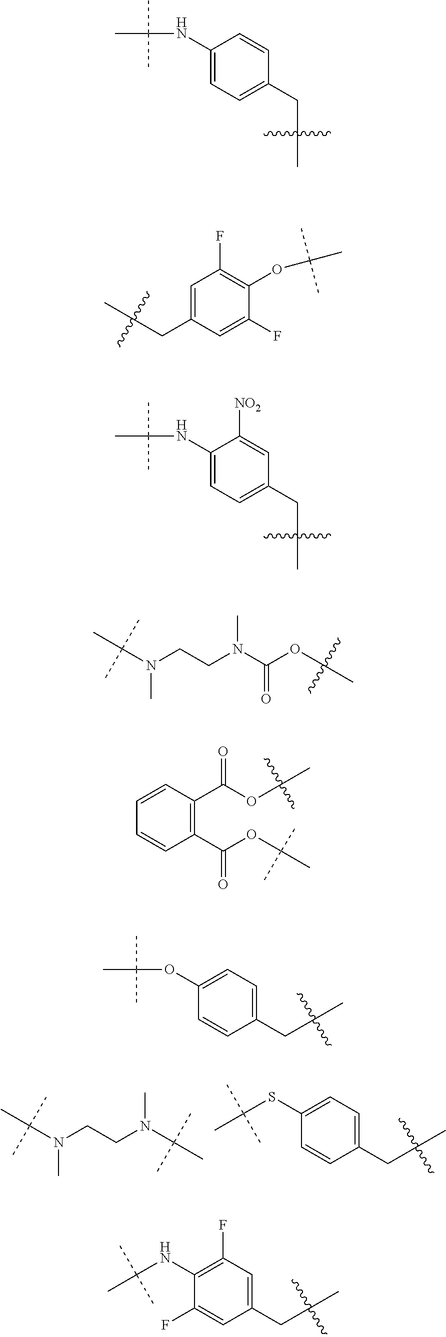

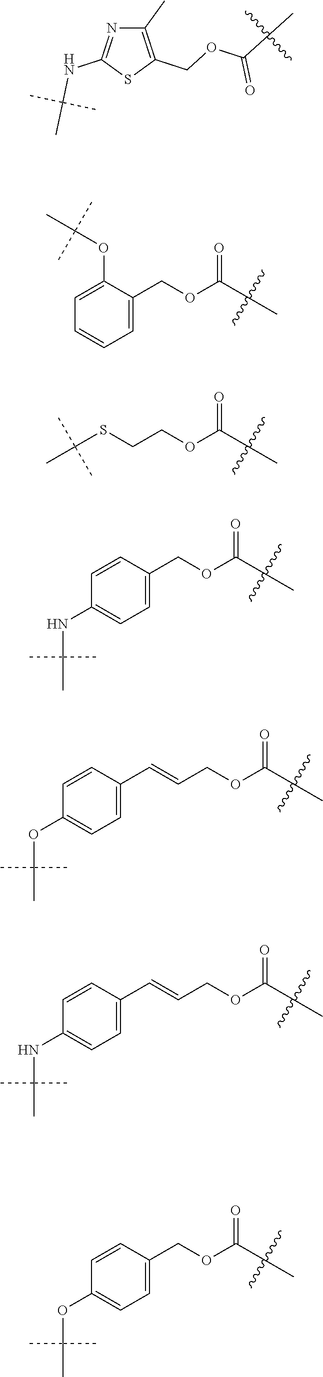

where the dotted line represents the point of attachment to the peptide and the wavy line represents the point of attachment to the rest of the molecule;
and the peptide is selected from:

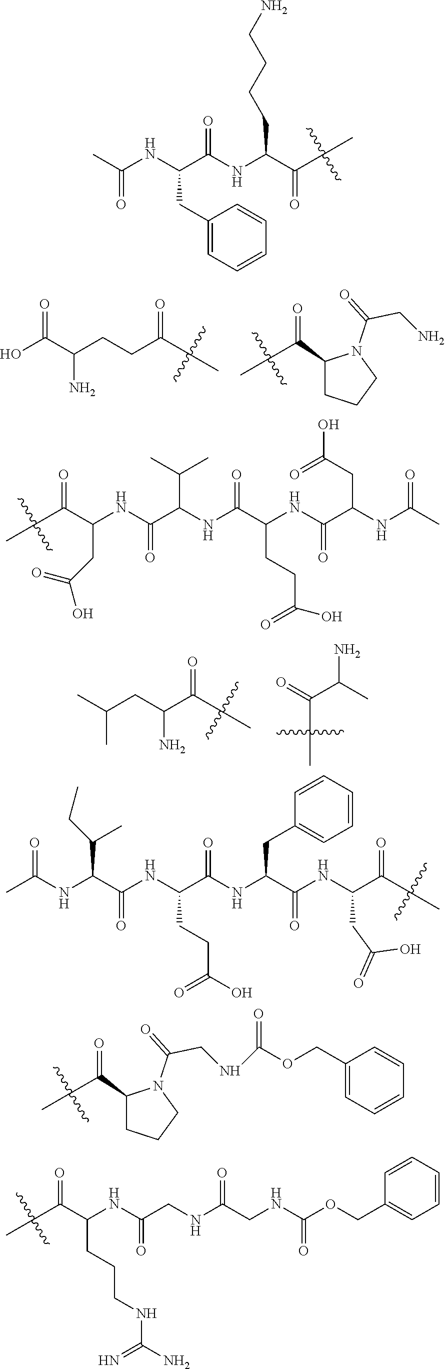

where the wavy line represents the point of attachment to the rest of the molecule,
or a pharmaceutically acceptable salt or solvate thereof.

In embodiments herein, the word “comprising” may be interpreted as requiring the features mentioned, but not limiting the presence of other features. Alternatively, the word “comprising” may also relate to the situation where only the components/features listed are intended to be present (e.g. the word “comprising” may be replaced by the phrases “consists of” or “consists essentially of”). It is explicitly contemplated that both the broader and narrower interpretations can be applied to all aspects and embodiments of the present invention. In other words, the word “comprising” and synonyms thereof may be replaced by the phrase “consisting of” or the phrase “consists essentially of” or synonyms thereof and vice versa.

The phrase, “consists essentially of” and its pseudonyms may be interpreted herein to refer to a material where minor impurities may be present. For example, the material may be greater than or equal to 90% pure, such as greater than 95% pure, such as greater than 97% pure, such as greater than 99% pure, such as greater than 99.9% pure, such as greater than 99.99% pure, such as greater than 99.999% pure, such as 100% pure.

As used herein, the singular forms “a,” “an,” and “the” include plural referents unless the context clearly dictates otherwise. Thus, for example, reference to “a compound” includes mixtures of two or more such compounds, reference to “a composition” includes mixtures of two or more such compositions, and the like.

A series of molecular radio afterglow probes (MRAPs) for cancer radiodynamic theranostics have been developed. Due to their unique activatable mechanisms, MRAPs can be used for ultrasensitive and specific detection of tumours.

The compounds of formula Ia and formula Ib (and salts and/or solvates thereof) may be particularly suited to use in in vivo cancer radio afterglowdynamic theranostics. In part, this is because the compounds of formula Ia and formula Ib contain at least one moiety that can be activated to provide radio afterglow, as described in more detail below. Further, the compounds of formula Ia and formula Ib may be particularly suited to the above application because of the presence of a cleavable moiety, as described in more detail below. Without wishing to be bound by theory, the addition of a cleavable moiety allows activation of the radio afterglow and radiodynamic functions of the compounds of formula Ia and formula Ib in the presence of a tumour biomarker, thus enabling precision cancer diagnosis and therapy (see Example 5). In addition, the compounds of formula Ia and formula Ib are particularly suited to the above application because of the presence of a tumour-targeting moiety, as described in more detail below. Without wishing to be bound by theory, the addition of a target group allows targeting towards αvβ integrin overexpressed in tumours (see Example 6). This thereby improves the specificity of the compounds in detecting cancers.

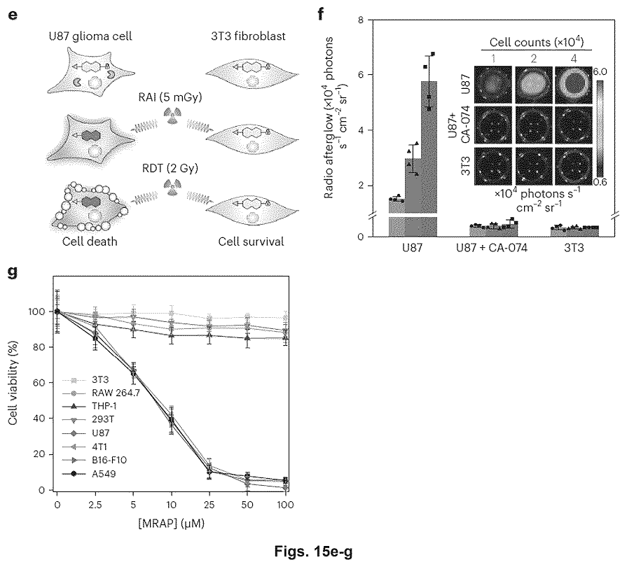

Thus, in certain embodiments disclosed herein, compounds of formula Ia and formula Ib may be designed to contain a tumour-targeting moiety, a radio theranostic moiety, and a cleavable moiety conjugated to a self-immolative linker (see FIG. 15a).

As mentioned above, the compounds of formula Ia and formula Ib contain at least one moiety that can be activated to provide radio afterglow. For example, the moiety may be an organic luminophore (IDPA).

When used herein, the term “PEG” is intended to refer to “polyethylene glycol”.

References herein (in any aspect or embodiment of the invention) to compounds of formulae Ia and Ib includes references to such compounds per se, to tautomers of such compounds, as well as to pharmaceutically acceptable salts and/or solvates of such compounds.

References to compounds of formulae Ia and Ib may also include reference to pharmaceutically functional derivatives of such compounds.

Pharmaceutically acceptable salts that may be mentioned include acid addition salts and base addition salts. Such salts may be formed by conventional means, for example by reaction of a free acid or a free base form of a compound of formula Ia or Ib with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of a compound of formula Ia or Ib in the form of a salt with another counter-ion, for example using a suitable ion exchange resin.

Examples of acid addition salts include acid addition salts formed with acetic, 2,2-dichloroacetic, adipic, alginic, aryl sulphonic acids (e.g. benzenesulphonic, naphthalene-2-sulphonic, naphthalene-1,5-disulphonic and p-toluenesulphonic), ascorbic (e.g. L-ascorbic), L-aspartic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, gluconic (e.g. D-gluconic), glucuronic (e.g. D-glucuronic), glutamic (e.g. L-glutamic), a-oxoglutaric, glycolic, hippuric, hydrobromic, hydrochloric, hydriodic, isethionic, lactic (e.g. (+)-L-lactic and (±)-DL-lactic), lactobionic, maleic, malic (e.g. (−)-L-malic), malonic, (±)-DL-mandelic, metaphosphoric, methanesulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, tartaric (e.g. (+)-L-tartaric), thiocyanic, undecylenic and valeric acids.

Particular examples of salts are salts derived from mineral acids such as hydrochloric, hydrobromic, phosphoric, metaphosphoric, nitric and sulphuric acids; from organic acids, such as tartaric, acetic, citric, malic, lactic, fumaric, benzoic, glycolic, gluconic, succinic, arylsulphonic acids; and from metals such as sodium, magnesium, or preferably, potassium and calcium.

As mentioned above, also encompassed by formula Ia and Ib are any solvates of the compounds and their salts. Preferred solvates are solvates formed by the incorporation into the solid state structure (e.g. crystal structure) of the compounds of the invention of molecules of a non-toxic pharmaceutically acceptable solvent (referred to below as the solvating solvent).

Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates. Particularly preferred solvates are hydrates, and examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, IN, USA, 1999, ISBN 0-967-06710-3.

“Pharmaceutically functional derivatives” of compounds of formula Ia or formula Ib as defined herein includes ester derivatives and/or derivatives that have, or provide for, the same biological function and/or activity as any relevant compound of the invention. Thus, for the purposes of this invention, the term also includes prodrugs of compounds of formula Ia or formula Ib.

The term “prodrug” of a relevant compound of formula Ia or formula Ib includes any compound that, following oral or parenteral administration, is metabolised in vivo to form that compound in an experimentally-detectable amount, and within a predetermined time (e.g. within a dosing interval of between 6 and 24 hours (i.e. once to four times daily)).

Prodrugs of compounds of formula Ia or formula Ib may be prepared by modifying functional groups present on the compound in such a way that the modifications are cleaved, in vivo when such prodrug is administered to a mammalian subject. The modifications typically are achieved by synthesizing the parent compound with a prodrug substituent. Prodrugs include compounds of formula Ia or formula Ib wherein a hydroxyl, amino, sulfhydryl, carboxyl or carbonyl group in a compound of formula Ia or formula Ib is bonded to any group that may be cleaved in vivo to regenerate the free hydroxyl, amino, sulfhydryl, carboxyl or carbonyl group, respectively.

Examples of prodrugs include, but are not limited to, esters and carbamates of hydroxyl functional groups, esters groups of carboxyl functional groups, N-acyl derivatives and N-Mannich bases. General information on prodrugs may be found e.g. in Bundegaard, H. “Design of Prodrugs” p. I-92, Elsevier, New York-Oxford (1985).

Compounds of formula Ia, as well as pharmaceutically acceptable salts, solvates and pharmaceutically functional derivatives of such compounds are, for the sake of brevity, hereinafter referred to together as the “compounds of formula Ia”. Compounds of formula Ib, as well as pharmaceutically acceptable salts and/or solvates of such compounds are, for the sake of brevity, hereinafter referred to together as the “compounds of formula Ib”.

Compounds of formula Ia or Ib may contain double bonds and may thus exist as E (entgegen) and Z (zusammen) geometric isomers about each individual double bond. All such isomers and mixtures thereof are included within the scope of the invention.

Compounds of formula Ia or Ib may exist as regioisomers and may also exhibit tautomerism. All tautomeric forms and mixtures thereof are included within the scope of the invention.

Compounds of formula Ia or Ib may contain one or more asymmetric carbon atoms and may therefore exhibit optical and/or diastereoisomerism. Diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation (i.e. a ‘chiral pool’ method), by reaction of the appropriate starting material with a ‘chiral auxiliary’ which can subsequently be removed at a suitable stage, by derivatisation (i.e. a resolution, including a dynamic resolution), for example with a homochiral acid followed by separation of the diastereomeric derivatives by conventional means such as chromatography, or by reaction with an appropriate chiral reagent or chiral catalyst all under conditions known to the skilled person. All stereoisomers and mixtures thereof are included within the scope of the invention.

Unless otherwise stated, the term “aryl” when used herein includes C₆₋₁₄ (such as C₆₋₁₀) aryl groups. Such groups may be monocyclic, bicyclic or tricyclic and have between 6 and 14 ring carbon atoms, in which at least one ring is aromatic. The point of attachment of aryl groups may be via any atom of the ring system. However, when aryl groups are bicyclic or tricyclic, they are linked to the rest of the molecule via an aromatic ring. C₆₋₁₄ aryl groups include phenyl, naphthyl and the like, such as 1,2,3,4-tetrahydronaphthyl, indanyl, indenyl and fluorenyl. Embodiments of the invention that may be mentioned include those in which aryl is phenyl.

Unless otherwise stated, the term “alkyl” refers to an unbranched or branched, cyclic, saturated or unsaturated (so forming, for example, an alkenyl or alkynyl) hydrocarbyl radical, which may be substituted or unsubstituted (with, for example, one or more halo atoms). Where the term “alkyl” refers to an acyclic group, it is preferably C₁₋₁₀ alkyl and, more preferably, C₁₋₆ alkyl (such as ethyl, propyl, (e.g. n-propyl or isopropyl), butyl (e.g. branched or unbranched butyl), pentyl or, more preferably, methyl). Where the term “alkyl” is a cyclic group (which may be where the group “cycloalkyl” is specified), it is preferably C₃₋₁₂ cycloalkyl and, more preferably, C₅₋₁₀ (e.g. C₅₋₇) cycloalkyl.

Further embodiments of the invention that may be mentioned include those in which the compound of formula Ia or formula Ib is isotopically labelled. However, other, particular embodiments of the invention that may be mentioned include those in which the compound of formula Ia or formula Ib is not isotopically labelled.

The term “isotopically labelled”, when used herein includes references to compounds of formula I in which there is a non-natural isotope (or a non-natural distribution of isotopes) at one or more positions in the compound. References herein to “one or more positions in the compound” will be understood by those skilled in the art to refer to one or more of the atoms of the compound of formula Ia or formula Ib. Thus, the term “isotopically labelled” includes references to compounds of formula Ia or formula Ib that are isotopically enriched at one or more positions in the compound.

The isotopic labelling or enrichment of the compound of formula Ia or formula Ib may be with a radioactive or non-radioactive isotope of any of hydrogen, carbon, nitrogen, oxygen, sulfur, fluorine, chlorine, bromine and/or iodine. Particular isotopes that may be mentioned in this respect include ²H, ³H, ¹¹C, ¹³C, ¹⁴C, ¹³N, ¹⁵N, ¹⁵O, ¹⁷O, ¹⁸O, ³⁵S, ¹⁸F, ³⁷CI, ⁷⁷Br, ⁸²Br and ¹²⁵I)

When the compound of formula Ia or formula Ib is labelled or enriched with a radioactive or nonradioactive isotope, compounds of formula Ia or formula Ib that may be mentioned include those in which at least one atom in the compound displays an isotopic distribution in which a radioactive or non-radioactive isotope of the atom in question is present in levels at least 10% (e.g. from 10% to 5000%, particularly from 50% to 1000% and more particularly from 100% to 500%) above the natural level of that radioactive or non-radioactive isotope.

In order to ensure that the compounds of formula Ia and formula Ib are delivered to cancer cells, R4 may represent

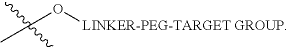

When used herein, the term

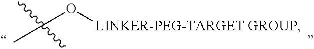

is intended to refer to a target group conjugated to PEG by way of a covalent bond, and the PEG conjugated to

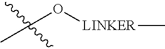

by way of a covalent bond. Examples of target groups have been provided above. Any suitable linker may be used in the current invention.

When used herein, the term “self-immolative linker” is a bi- , tri- or tetra-functional chemical moiety which is capable of covalently linking together two to four spaced chemical moieties into a normally stable tri-, tetra or quintapartite molecule, that can release at least one of the spaced chemical moieties from the stable molecule by means of cleavage of a peptide moiety, and following said cleavage, the self-immolative linker can spontaneously cleave from the remainder of the molecule to release the other spaced chemical moiety(ies). An example of this cleavage process discussed in Example 5. Any suitable self-immolative linker may be used in the current invention.

When used herein, the term “-SELF IMMOLATIVE LINKER-PEPTIDE” is intended to refer to a peptide moiety conjugated to a self-immolative linker by way of a covalent bond. For example, the peptide moiety may be a biomarker reactive moiety.

In particular embodiments that may be mentioned herein, the compound, or pharmaceutically acceptable salt or solvate thereof, may have formula Ia. In such embodiments that may be mentioned herein, R may represent:

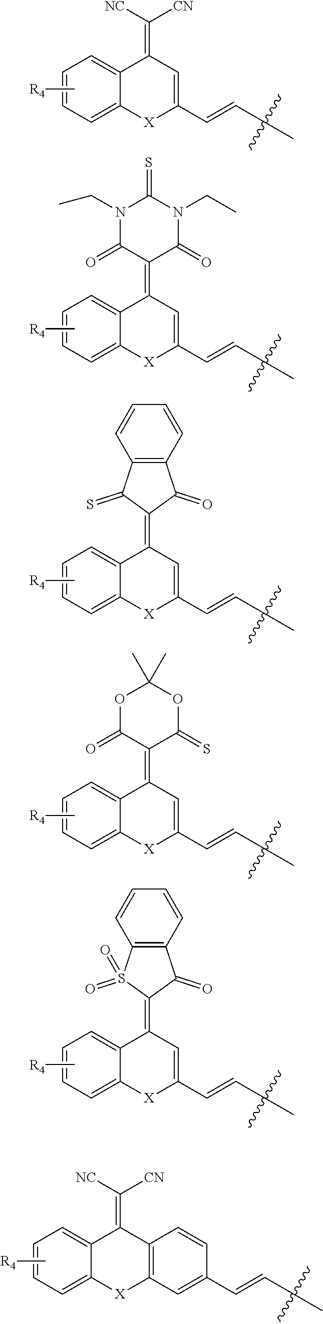

where the wiggly line represents the point of attachment to the rest of the molecule; and X represents NEt, O, S or Se, such as 0, S or Se, such as S. In further embodiments that may be mentioned herein, R may represent:

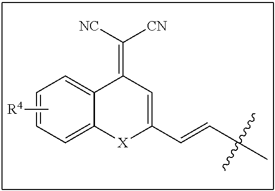

where the wiggly line represents the point of attachment to the rest of the molecule; and X represents 0, S or Se, such as S.

In particular embodiments that may be mentioned herein, R₄ may be independently selected from:

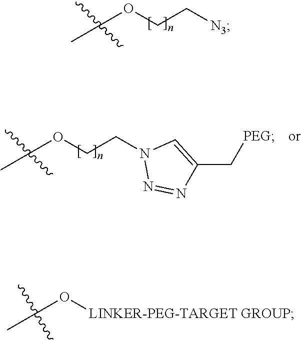

where:
   the wiggly line represents the point of attachment to the rest of the molecule;
   PEG is PEG1K, PEG2K, or PEG5K; and
   n is 0 to 11.

In further embodiments that may be mentioned herein, R₄ may be independently selected from:

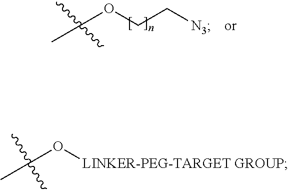

where:
   the wiggly line represents the point of attachment to the rest of the molecule;
   PEG is PEG1K, PEG2K, or PEG5K; and
   n is 3 to 5, such as 4.

In particular embodiments that may be mentioned herein, when present, LINKER may represent:

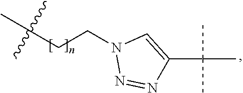

where n is 3 to 5, such as 4, and the wiggly line represents the point of attachment to the oxygen atom and the dotted line represents the point of attachment to the TARGET GROUP.

In particular embodiments that may be mentioned herein, when present, TARGET GROUP may represent:

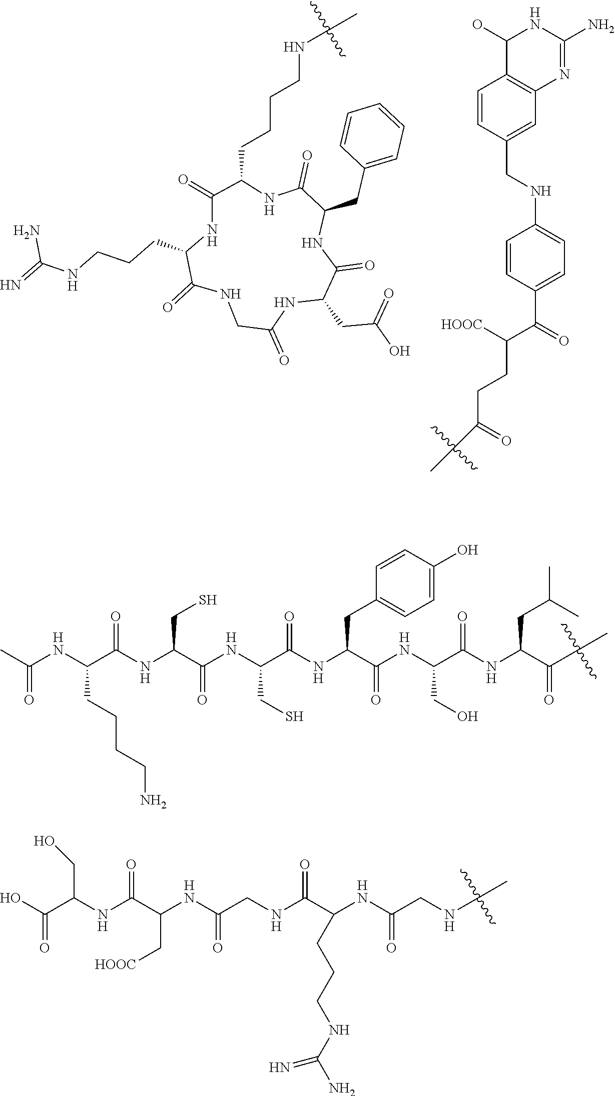 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O ; *C(=O)CCC(C(=O)O)C(=O)c1ccc(NCc2ccc3c(c2)N=C(N)NC3O)cc1 ; *C(=O)[C@H](CC(C)C)NC(=O)[C@H](CO)NC(=O)[C@H](Cc1ccc(O)cc1)NC(=O)[C@H](CS)NC(=O)[C@H](CS)NC(=O)[C@H](CCCCN)NC(C)=O ; *NCC(=O)NC(CCCNC(=N)N)C(=O)NCC(=O)NC(CC(=O)O)C(=O)NC(CO)C(=O)O]

where the wiggly line represents the point of attachment to the rest of the molecule.

In further embodiments that may be mentioned herein, when present, TARGET GROUP may represent:

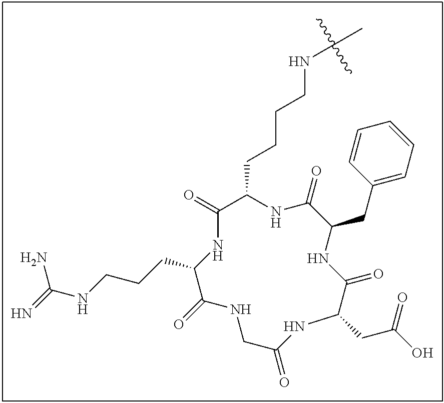 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O]

where the wiggly line represents the point of attachment to the rest of the molecule.

In particular embodiments that may be mentioned herein, R₁ may represent H or I.

In particular embodiments that may be mentioned herein, R₂ may represent H.

In particular embodiments that may be mentioned herein, R₃ may either represent:

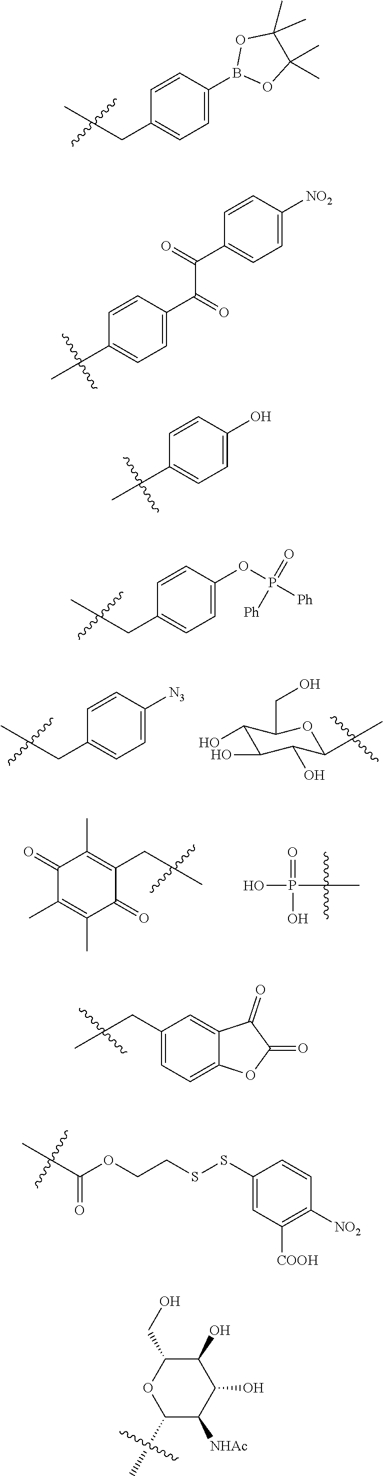

the wavy line represents the point of attachment to the rest of the molecule;
or R₃ may represent -SELF INMMOLATIVE LINKER-PEPTIDE, where the self immolative linker is:

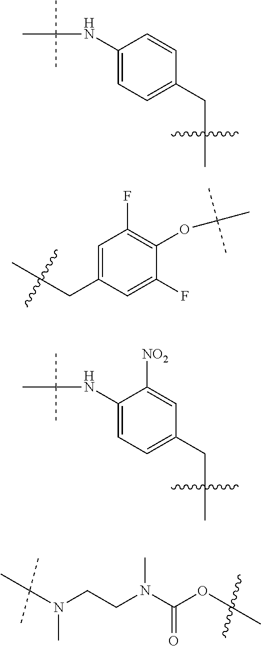

where the dotted line represents the point of attachment to the peptide and the wavy line represents the point of attachment to the rest of the molecule.

In particular embodiments that may be mentioned herein, R₃ may represent -SELF IMMOLATIVE LINKER-PEPTIDE, where the self immolative linker may be:

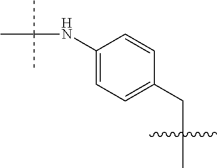

where the dotted line represents the point of attachment to the peptide and the wavy line represents the point of attachment to the rest of the molecule.

In particular embodiments that may be mentioned herein, when R₃ represents -SELF IMMOLATIVE LINKER-PEPTIDE, the peptide may be selected from:

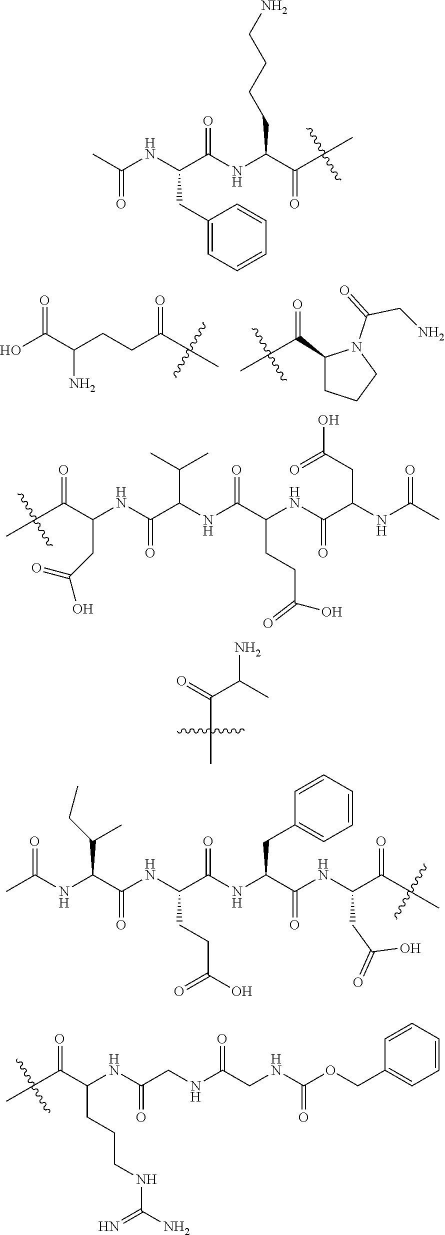

where the wavy line represents the point of attachment to the rest of the molecule.

In further embodiments that may be mentioned herein, when R₃ represents -SELF IMMOLATIVE LINKER-PEPTIDE, the peptide may be:

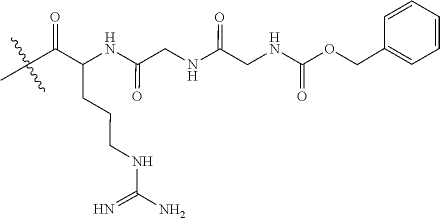 [SMILES: *C(=O)C(CCCNC(=N)N)NC(=O)CNC(=O)CNC(=O)OCc1ccccc1]

where the wavy line represents the point of attachment to the rest of the molecule.

As shown above, each of R₃ group may represent a biomarker reactive moiety or a biomarker reactive moiety (i.e. a peptide moiety) conjugated to a self-immolative linker. When used herein, the term “radio theranostic moiety” is intended to refer to a substituent group that does not emit radio afterglow and radiodynamic signals due to the presence of a biomarker reactive moiety or a biomarker reactive moiety conjugated to a self-immolative linker by way of a covalent bond, but which is capable of emitting radio afterglow and radiodynamic signals following cleavage of said covalent bond. In other words, the compounds of formula Ia and Ib contain a radio theranostic moiety that initially does not emit radio afterglow and radiodynamic signals because the radio theranostic moiety is in a “caged” state due to a covalent bond reducing the donation of electrons to the radio theranostic moiety system. In the presence of a suitable biomarker, the relevant covalent bond is cleaved (i.e. the covalent bond to the peptide moiety when bonded to it directly or the covalent bond to the self-immolative linker when bonded to this directly), thereby providing a free radio theranostic moiety. This cleavage “uncages” the radio theranostic moiety and results in increased electron donation into the radio theranostic moiety system, thus making the radio theranostic moiety emit radio afterglow and radiodynamic signals.

Therefore, the compounds of formula Ia and Ib, are able to selectively activate radio afterglow signals in the presence of a biomarker reactive moiety that is indicative of cancer. As will be appreciated, the biomarkers referred to herein are intended to be ones that may be upregulated in cancer cells. Such biomarkers may include enzymes, reactive oxygen species and reactive nitrogen species (RNS). It will be appreciated that other biomarker reactive moieties other than those disclosed herein may be used in the compounds of formula Ia and formula Ib and so the compounds of formula Ia and formula Ib are not exclusively limited to the biomarker reactive moieties disclosed herein.

In particular embodiments that may be mentioned herein,
R₁ may be I;
R₂ may be H;
R may be:

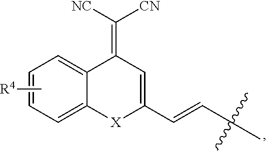

where the wavy line represents the point of attachment to the rest of the molecule;
R₄ may be

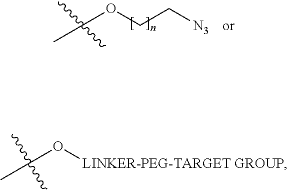

where LINKER may be

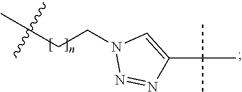

n may be 4;
PEG may be PEG2K;

Target Group may be

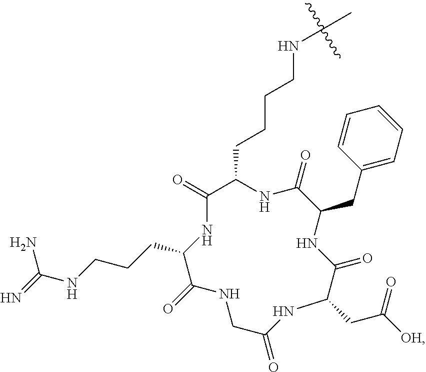 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O]

where the wiggly line represents the point of attachment to the rest of the molecule;
R₃ may be -SELF IMMOLATIVE LINKER-PEPTIDE, where the self immolative linker is

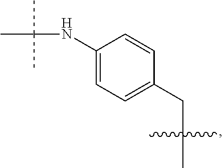

where the dotted line represents the point of attachment to the peptide and the wavy line represents the point of attachment to the rest of the molecule; and
the peptide is

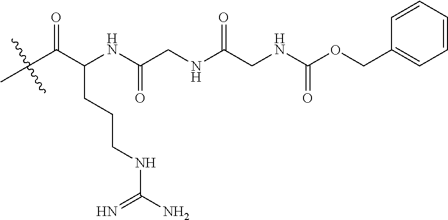 [SMILES: *C(=O)C(CCCNC(=N)N)NC(=O)CNC(=O)CNC(=O)OCc1ccccc1]

where the wavy line represents the point of attachment to the rest of the molecule.

In a second aspect of the invention, there is provided a method for detection of cancer in vivo, the method comprising the following steps:
(ai) administering a compound of formula Ia or Ib, or pharmaceutically acceptable salt or solvate thereof, according to the first aspect of the invention to a subject; and
(aii) detecting any chemiluminescence, wherein the presence of cancer in vivo is indicated by chemiluminescence.

In embodiments of the invention, the second aspect may further comprise treating the subject for cancer if this is detected. Any possible cancer therapy may be used according to the prognosis and discretion of the attending physician, including the therapy described in the third aspect of the invention below.

The second aspect of the invention may also be expressed as a compound of formula Ia or Ib, or pharmaceutically acceptable salt or solvate thereof, according to the first aspect of the invention for use in the detection of cancer in vivo. Where the compound of formula Ia or Ib, or pharmaceutically acceptable salt or solvate thereof is administered to a subject and detecting any chemiluminescence, wherein the presence of cancer in vivo is indicated by chemiluminescence.

Further, the second aspect of the invention may be expressed as use of a compound of formula Ia or Ib, or pharmaceutically acceptable salt or solvate thereof, according to the first aspect of the invention for use in the preparation of a medicament for detecting cancer. Where the compound of formula Ia or Ib, or pharmaceutically acceptable salt or solvate thereof is to be administered to a subject and detecting any chemiluminescence, wherein the presence of cancer in vivo is indicated by chemiluminescence.

As will be appreciated, in this detection of cancer, the compounds of formula Ia or Ib, or pharmaceutically acceptable salts or solvates thereof are chosen to provide self-illuminating signal in vivo (e.g. due to the untrapping of a chemiliminescent moiety in the body).

When used herein, the term “self illuminating signal” is intended to include radio afterglow signals and chemiluminescence.

In a third aspect of the invention, there is provided a method of treating cancer, the method comprising the following steps:
(bi) administering a compound of formula Ia or Ib, or pharmaceutically acceptable salt or solvate thereof, according to the first aspect of the invention to a subject; and
(bii) subjecting the subject to X-ray irradiation.

As will be appreciated, the X-ray irradiation may be helpfully targeted to areas that display chemiluminescence when the compounds of formula Ia or Ib, or pharmaceutically acceptable salts or solvates thereof are selected to provide chemiluminescence in vivo. However, this is only a desirable characteristic and is not needed for the treatment to work.

In addition, it is noted that X-ray irradiation has two properties:
i) X-ray induced afterglow imaging through singlet oxygen generation;
ii) ii) X-ray activated therapy through reactive oxygen species (ROS).

The third aspect of the invention may also be expressed as a compound of formula Ia or Ib, or pharmaceutically acceptable salt or solvate thereof, according to the first aspect of the invention for use in the treatment of cancer. Where the compound of formula Ia or Ib, or pharmaceutically acceptable salt or solvate thereof is administered to a subject and the subject is then subjected to X-ray irradiation.

Further, the third aspect of the invention may be expressed as use of a compound of formula Ia or Ib, or pharmaceutically acceptable salt or solvate thereof, according to the first aspect of the invention for use in the preparation of a medicament for treating cancer. Where the compound of formula Ia or Ib, or pharmaceutically acceptable salt or solvate thereof is to be administered to a subject and the subject is then subjected to X-ray irradiation.

In some embodiments that may be mentioned herein, the compound of formula Ia or formula Ib, or salt or solvate thereof, may be a compound in which R₄ is

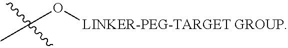

Compounds of formula Ia and formula Ib may be administered by any suitable route, but may particularly be administered orally, intravenously, intramuscularly, cutaneously, subcutaneously, transmucosally (e.g. sublingually or buccally), rectally, transdermally, nasally, pulmonarily (e.g. tracheally or bronchially), topically, by any other parenteral route, in the form of a pharmaceutical preparation comprising the compound in a pharmaceutically acceptable dosage form. Particular modes of administration that may be mentioned include oral, intravenous, cutaneous, subcutaneous, nasal, intramuscular or intraperitoneal administration.

Compounds of formula Ia and formula Ib will generally be administered as a pharmaceutical formulation in admixture with a pharmaceutically acceptable adjuvant, diluent or carrier, which may be selected with due regard to the intended route of administration and standard pharmaceutical practice. Such pharmaceutically acceptable carriers may be chemically inert to the active compounds and may have no detrimental side effects or toxicity under the conditions of use. Suitable pharmaceutical formulations may be found in, for example, Remington The Science and Practice of Pharmacy, 19th ed., Mack Printing Company, Easton, Pennsylvania (1995). For parenteral administration, a parenterally acceptable aqueous solution may be employed, which is pyrogen free and has requisite pH, isotonicity, and stability. Suitable solutions will be well known to the skilled person, with numerous methods being described in the literature. A brief review of methods of drug delivery may also be found in e.g. Langer, Science (1990) 249, 1527.

Otherwise, the preparation of suitable formulations may be achieved routinely by the skilled person using routine techniques and/or in accordance with standard and/or accepted pharmaceutical practice.

The amount of compound of formula Ia or formula Ib in any pharmaceutical formulation used in accordance with the present invention will depend on various factors, such as the condition to be diagnosed, the particular patient that is the subject of the diagnosis, as well as the compound(s) which is/are employed. In any event, the amount of compound of formula Ia or formula Ib in the formulation may be determined routinely by the skilled person.

For example, a solid oral composition such as a tablet or capsule may contain from 1 to 99% (w/w) diagnostic ingredient; from 0 to 99% (w/w) diluent or filler; from 0 to 20% (w/w) of a disintegrant; from 0 to 5% (w/w) of a lubricant; from 0 to 5% (w/w) of a flow aid; from 0 to 50% (w/w) of a granulating agent or binder; from 0 to 5% (w/w) of an antioxidant; and from 0 to 5% (w/w) of a pigment. A controlled release tablet may in addition contain from 0 to 90% (w/w) of a release-controlling polymer.

A parenteral formulation (such as a solution or suspension for injection or a solution for infusion) may contain from 1 to 50% (w/w) diagnostic ingredient; and from 50% (w/w) to 99% (w/w) of a liquid or semisolid carrier or vehicle (e.g. a solvent such as water); and 0-20% (w/w) of one or more other excipients such as buffering agents, antioxidants, suspension stabilisers, tonicity adjusting agents and preservatives.

Depending on the condition to be diagnosed, and the patient to be diagnosed, as well as the route of administration, compounds of formula Ia or formula Ib may be administered at varying diagnostically effective doses to a subject in need thereof.

However, the dose administered to a mammal, particularly a human, in the context of the current invention should be sufficient to effect a therapeutic response in the mammal over a reasonable timeframe. One skilled in the art will recognise that the selection of the exact dose and composition and the most appropriate delivery regimen will also be influenced by inter alia the pharmacological properties of the formulation, the nature and severity of the condition being diagnosed, and the physical condition and mental acuity of the recipient, as well as the properties of the specific compound, the age, condition, body weight, sex and response of the patient to be diagnosed, and the suspected stage/severity of the disease.

Administration may be continuous or intermittent (e.g. by bolus injection). The dosage may also be determined by the timing and frequency of administration. In the case of oral or parenteral administration the dosage can vary from about 0.01 mg to about 1000 mg per day of a compound of formula Ia or formula Ib.

In any event, the medical practitioner, or other skilled person, will be able to determine routinely the actual dosage, which will be most suitable for an individual subject. The above-mentioned dosages are exemplary of the average case; there can, of course, be individual instances where higher or lower dosage ranges are merited, and such are within the scope of this invention.

The term “cancer” will be understood by those skilled in the art to include conditions such as, but not limited to, adrenal cancer, anal cancer, bile duct cancer, bladder cancer, bone cancer, brain tumours, CNS tumours, breast cancer, Castleman disease, cervical cancer, colon cancer, rectum cancer, endometrial cancer, esophagus cancer, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal cancer, hypopharyngeal cancer, leukemia (e.g. acute lymphocytic, acute myeloid, chronic lymphocytic, chronic myeloid, chronic myelomonocytic), liver cancer, lung cancer (e.g. small cell or non-small cell), lung carcinoid tumour, lymphoma (e.g. of the skin), malignant mesothelioma, multiple myeloma, myelodysplastic syndrome, nasal cavity cancer, paranasal sinus cancer, nasopharyngeal cancer, neuroblastoma, non-Hodgkin lymphoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, and Wilms tumour.

Particular cancers that may be mentioned herein may be in the form of solid tumours (such as adrenal cancer, anal cancer, bile duct cancer, bladder cancer, bone cancer, brain tumours, CNS tumours, breast cancer, Castleman disease, cervical cancer, colon cancer, rectum cancer, endometrial cancer, esophagus cancer, eye cancer, gallbladder cancer, gastrointestinal carcinoid tumors, gastrointestinal stromal tumor (GIST), gestational trophoblastic disease, Hodgkin disease, Kaposi sarcoma, kidney cancer, laryngeal cancer, hypopharyngeal cancer, liver cancer, lung cancer (e.g. small cell or non-small cell), lung carcinoid tumour, malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer, Waldenstrom macroglobulinemia, and Wilms tumour).

In particular embodiments that may be mentioned herein, the cancer may be one or more of the following: brain cancer, adrenal cancer, anal cancer, bile duct cancer, bladder cancer, bone cancer, CNS tumours, breast cancer, Castleman disease, cervical cancer, colon cancer, rectum cancer, endometrial cancer, esophagus cancer, eye cancer, gallbladder cancer, gastrointestinal carcinoid tumors, gastrointestinal stromal tumor, gestational trophoblastic disease, Hodgkin disease, Kaposi sarcoma, kidney cancer, laryngeal cancer, hypopharyngeal cancer, liver cancer, lung cancer, lung carcinoid tumour, malignant mesothelioma, multiple myeloma, myelodysplastic syndrome, nasal cavity cancer, paranasal sinus cancer, nasopharyngeal cancer, neuroblastoma, oral cavity cancer, oropharyngeal cancer, osteosarcoma, ovarian cancer, pancreatic cancer, penile cancer, pituitary tumours, prostate cancer, retinoblastoma, rhabdomyosarcoma, salivary gland cancer, sarcoma, skin cancer (basal and squamous cell, melanoma, Merkel cell), small intestine cancer, stomach cancer, testicular cancer, thymus cancer, thyroid cancer, uterine sarcoma, vaginal cancer, vulvar cancer.

For the avoidance of doubt, in the context of the present invention, the term “treatment” includes references to therapeutic or palliative treatment of patients in need of such treatment, as well as to the prophylactic treatment and/or diagnosis of patients which are susceptible to the relevant disease states.

The terms “patient” and “patients” include references to mammalian (e.g. human) patients. As used herein the terms “subject” or “patient” are well-recognized in the art, and, are used interchangeably herein to refer to a mammal, including dog, cat, rat, mouse, monkey, cow, horse, goat, sheep, pig, camel, and, most preferably, a human. In some embodiments, the subject is a subject in need of treatment or a subject with a disease or disorder. However, in other embodiments, the subject can be a normal subject. The term does not denote a particular age or sex. Thus, adult and newborn subjects, whether male or female, are intended to be covered.

The term “effective amount” refers to an amount of a compound, which confers a therapeutic effect on the treated patient (e.g. sufficient to treat or prevent the disease). The effect may be objective (i.e. measurable by some test or marker) or subjective (i.e. the subject gives an indication of or feels an effect).

Further aspects and embodiments of the invention will now be discussed by reference to the following non-limiting examples.

### EXAMPLES

### Materials

The chemicals were purchased from Sigma-Aldrich, TCI and BLDpharm without further purification. All the deuterated solvents (dimethyl sulfoxide-d6 (DMSO-d₆), CDCl₃ and CD₃OD) were ordered from Cambridge Isotope Laboratories. ESR spin-trapping agent (2,2,6,6-tetramethylpiperidine (TEMP)) was purchased from Sigma-Aldrich. Cyclic arginine-glycine-aspartate (cRGD) peptide and CatB inhibitor (CA-074) were obtained from MedChemExpress. ROS sensors (SOSG, DHR123 and HPF) were purchased from Thermo Fisher Scientific or Sigma-Aldrich. Enzymes ALP, β-Gal, NTR, GGT and CatB were purchased from Sigma-Aldrich, whereas APN, furin and uPA were purchased from R&D Systems. Anti-cleaved caspase-3 antibody, anti-caspase-1 antibody and Alexa Fluor 594 secondary antibody were purchased from Abcam and Affbiotech. 4′,6-Diamidino-2-phenylindole (DAPI), TUNEL assay and H&E were purchased from Servicebio or Abcam company. Cell Counting Kit-8 was ordered from Dojindo Molecular Technologies.

### Ethical Requirements

All the animal experimental procedures were conducted according to the guidelines of the Regional Ethics Committee for Animal Experiments and the Care Regulations approved by the Institutional Animal Care and Use Committee of Fuzhou University. According to the guidelines, mice were sacrificed when the largest tumour size limit is 1.5 cm (at the largest dimension) or 2,000 mm³ (for a subcutaneous tumour model) or a weight loss of 20% (for an orthotopic tumour model).

### General Procedure for the Preparation of the Stock Solution

DPA_{X} and IDPA_{X} (X=N, O, Su and Se) and the activatable probes (MRAP and cRGD-free MRAP) were stocked in 0.01 M PBS (pH 7.4, 50% DMSO) at room temperature. The enzymes (ALP, j-gal, furin, NTR, GGT, CatB, APN and uPA) were stocked in distilled water at −80° C.

### Analytical Techniques

Nuclear magnetic resonance (NMR) spectroscopy

The ¹H and ¹³C NMR spectra were measured with a Bruker BBFO1 400 nuclear magnetic resonance instrument.

### Liquid Chromatography-Mass Spectrometry (LC-MS) and High-Resolution Mass Spectrometry (HR-MS)

Liquid chromatography-mass spectrometry and high-resolution mass spectrometry spectra were conducted on a Thermo Scientific LTQ XL MS and Waters Xevo G2-X2 MS instruments, respectively.

### MALDI Time-of-Flight Mass Spectrometry (TOF-MS)

MALDI TOF-MS analyses were performed on a Bruker ultraflex TOF/TOF instrument.

### High-Performance Liquid Chromatography (HPLC) Purification and Analyses

HPLC purification and analyses were performed with an Agilent 1260 system using methanol/H₂O or acetonitrile/H₂O as an eluent. X-ray irradiation was conducted with MOXTEK MAGPRO (Moxtek, Inc. 452 West 1260 North Orem, UT 84057) and the dose was calibrated by RAYSAFE R/F X2 X (FLUKE, USA). White-light irradiation was used by a xenon lamp PLS-SEX300D (Beijing Perfectlight Technology Co., Ltd.).

### Absorption and Fluorescence Spectroscopy

The absorption and fluorescence spectra were recorded with a GENESYS 50 (Thermo Fisher Scientific) and Cary Eclipse fluorescence spectrophotometer (Agilent Technologies), respectively.

### Electron Paramagnetic Resonance (EPR) Spectroscopy

EPR spectroscopy was conducted with a Bruker A300 ESR spectrometer (Billerica, MA, USA).

### Radio Afterglow and Bioluminescence Imaging

Radio afterglow and bioluminescence imaging were recorded with an IVIS spectrum imaging system (IVIS Lumina XRMS, PerkinElmer).

### Procedure for Cell Assays and Cell Viability

All the cells were purchased from the American Type Culture Collection, including CatB-negative cells (mouse embryonic fibroblasts 3T3 cells, RAW 264.7, 293-T and human monocytic THP-1 cells) and CatB-positive cells (human U87 glioblastoma cells, U87-luciferase cells, A549 lung epithelial cells and 4T1 and melanoma B16-F10). All the cells were cultured

in Dulbecco's modified Eagle medium (Gibco) or RPMI 1640 medium containing 10.0% foetal bovine serum (Gibco) and 0.1% ciprofloxacin in a humidified atmosphere containing 5.0% CO2 and 95.0% air at 37° C. To study the cell viability of IDPA_{Su}, U87 cells (1×104 per well) were seeded on 96-well plates and incubated for 24 h. IDPA_{Su} at different concentrations (0, 2.5, 5.0, 10.0, 25.0, 50.0 and 100.0 μM) were added to the U87 cell medium for 12 h incubation. After incubation, 96-well plates were covered with chicken breast tissues with different thicknesses (0, 5, 8 and 15 cm), followed by X-ray irradiation. The X-ray setup was optimized to ensure the cells in all the wells received similar dose of X-ray irradiation (2 Gy) before chicken breast tissue coverage (0 cm). After irradiation, the cells were further incubated for 12 h, followed by the colorimetric Cell Counting Kit-8 assay. Briefly, Cell Counting Kit-8 solution (10 l, Dojindo Molecular Technologies) was added into the cell medium (100 l) for 2 h incubation, and the absorbance of medium at 450 nm was recorded using a microplate reader (ReadMax 1900). The cells without IDPA_{Su} incubation and X-ray irradiation were set as control. The cell viability (%) was calculated by the absorbance of treated cells over that of the control cells. To test the cell viability of activatable MRAP, U87 and 3T3 cells (1×10⁴ per well) were seeded on 96-well plates and incubated for 24 h. The inhibitor group was pre-treated with a CatB inhibitor (CA-074, 1 mM) for 10 h incubation. MRAP at different concentrations (0, 2.5, 5.0, 10.0, 25.0, 50.0 and 100.0 M) were added to the U87 and 3T3 cell medium for 12 h incubation. The subsequent steps were similar to the measurement of cell viability of IDPA_{Su}. Other CatB-positive and CatB-negative cells were conducted with the same protocol.

The cell viability was examined with ReadMax 1900 and infinite M200 PRO (TECAN).

### Statistics and Reproducibility

The results in all the experiments were presented as mean±standard deviation (s.d.) (n >3). All the radio afterglow and bioluminescence signals were quantified with the region-of-interest analysis using Living Image 4.3 software. The tumour area and immunofluorescence and SOSG intensities were quantified using ImageJ v1.51a software. Investigators were blinded to group allocation during the experiments. Statistical calculation of experimental data was analysed using the one-way analysis of variance with a Tukey post-hoc test (more than three independent groups) or a two-tailed Student's t-test (two independent groups) after normality and equal variance tests. The p values (<0.05) were considered statistically significant: *p<0.05, **p<0.01, ***p<0.001, ****p<0.0001. A statistical analysis was performed using Origin 2021 or GraphPad Prism 8.0 software.

### Example 1. Synthesis of Organic Luminophores

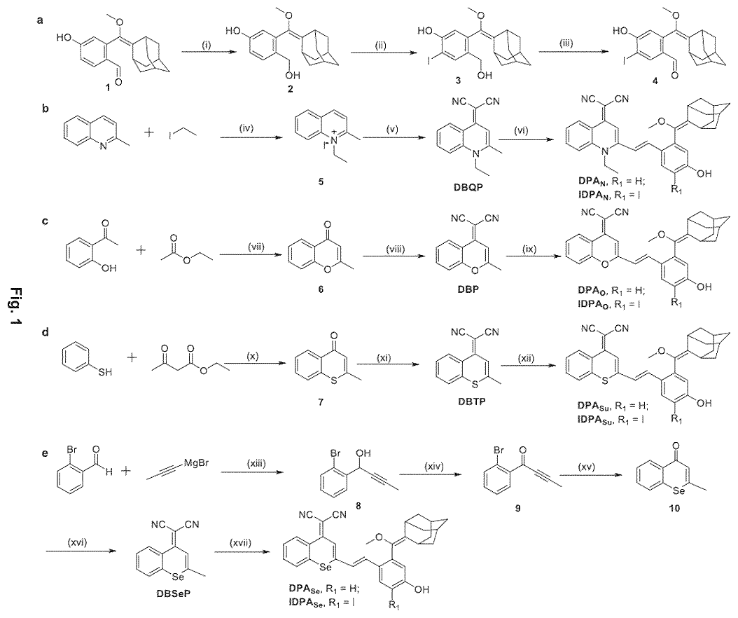

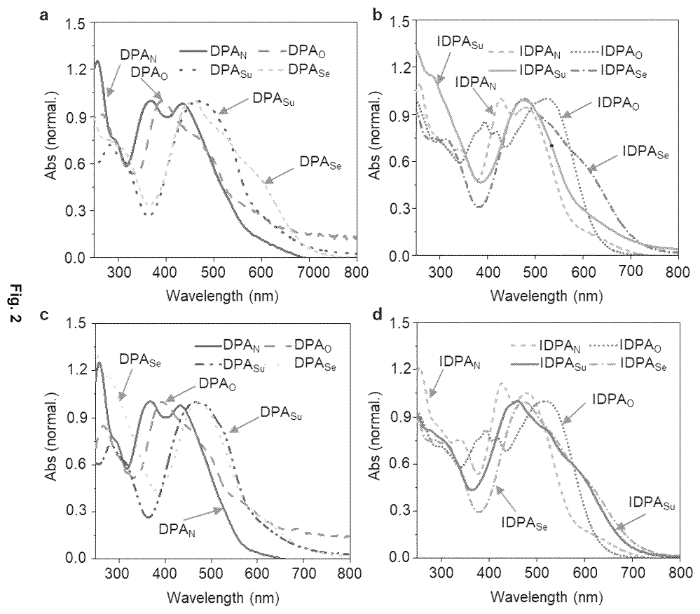

The radio afterglow dynamic luminophores were synthesized based on the acceptor-phenoxy-adamantylidene scaffold via a convergent approach (FIG. 1). First, the acceptors including dicyanomethylene-4H-quinoline (DBQ), dicyanomethylene-4H-benzopyran (DBP), dicyanomethylene-4H-benzothiopyran (DBTP) and dicyanomethylene-4H-benzoselenopyran (DBSeP) were synthesized (FIGS. 1b-e). Then, these acceptors were linked to phenoxy-adamantylidene with or without the heavy atom (iodine), respectively, leading to DBQ-phenoxy-adamantylidene (DPA_{N}), DBP-phenoxy-adamantylidene (DPA_{O}), DBTP-phenoxy-adamantylidene (DPA_{Su}), DBSeP-phenoxy-adamantylidene (DPA_{Se}) and the corresponding iodine-containing counterparts, namely, IDPA_{X} (X=N, O, Su and Se) (FIG. 1a). All the compounds were confirmed by nuclear magnetic resonance and liquid chromatography-mass spectrometry.

### Synthesis of Compound 2

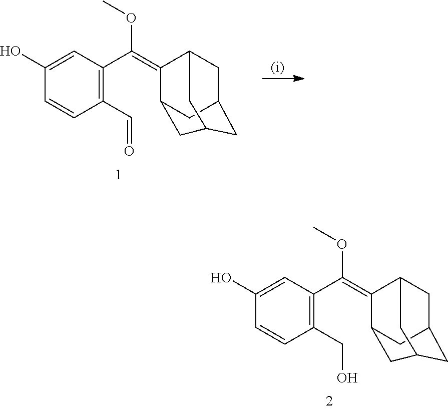 [SMILES: COC(=C1C2CC3CC(C2)CC1C3)c1cc(O)ccc1C=O ; "(i)" ; COC(=C1C2CC3CC(C2)CC1C3)c1cc(O)ccc1CO]

The phenol-adamantylidene derivative compound 1 was successfully synthesized according to previous literature (Gnaim, S. & Shabat, D., J. Am. Chem. Soc. 2017, 139, 10002-10008). The sodium borohydride (0.755 g, 20 mmol) was added portionwise over a period of 10 min to a solution of compound 1 (1.49 g, 5 mmol) in MeOH (20 ml) and stirred for 30 min at room temperature. After completing the reaction by thin layer chromatography (TLC) monitoring, the reaction mixture was concentrated in vacuo to remove methanol and extracted with ethyl acetate (EtOAc, 3×50 ml) and brine (3×50 ml). The organic layer was dried on anhydrous Na₂SO₄. The residue was obtained in vacuo and purified by flash column chromatography to give a white solid compound 2 (1.5 g, 100% yield).

¹H NMR (400 MHz, CDCl₃): δ 7.28 (d, J=2 Hz, 1H), 6.77 (dd, J=2.8, 8.4 Hz, 1H), 6.71 (d, J=2.8 Hz, 1H), 4.30-4.81 (m, 2H), 3.32 (s, 3H), 3.28 (s, 1H), 2.28 (s, 1H), 1.72-1.99 (m, 12H). ¹³C NMR (101 MHz, CDCl₃): δ 155.3, 141.8, 136.3, 133.8, 132.3, 131.0, 118.2, 115.2, 64.1, 57.8, 50.9, 39.3, 37.1, 32.6, 29.9, 28.4. MS (ESI⁻): m/z=299.1 [M−H]⁻.

### Synthesis of Compound 3

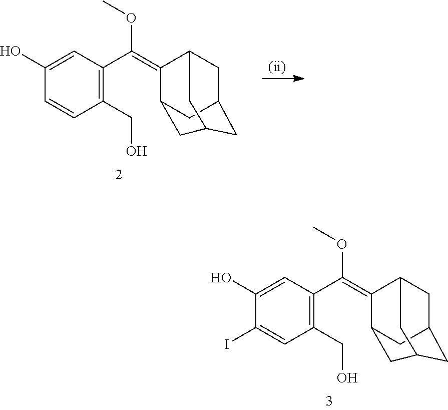 [SMILES: COC(=C1C2CC3CC(C2)CC1C3)c1cc(O)ccc1CO ; "(ii)" ; COC(=C1C2CC3CC(C2)CC1C3)c1cc(O)c(I)cc1CO]

To a solution of compound 2 (1.2 g, 4 mmol) and NaOH (240 mg, 6 mmol) in MeOH (25 ml) was added dropwise ICl (649 mg, 4 mmol) at 0° C. and stirred for 1 h. Upon completing the reaction, the mixture was quenched with saturated Na₂S₂O₃ (30 ml) and stirred for 10 min. The methanol was removed in vacuo and the residue was extracted with EtOAc (3×50 ml) and saturated NH₄Cl (2×100 ml). The organic phase was collected and dried with anhydrous Na₂SO₄, followed by concentration. The residue was purified by column chromatography on silica gel using hexane/EtOAc as the eluent to afford compound 3 (1.28 g, 75% yield) as a white solid.

¹H NMR (400 MHz, CDCl₃): δ 7.74 (s, 1H), 6.80 (s, 1H), 5.6 (s, 1H, OH), 4.50 (br, 2H), 3.29 (s, 3H), 3.24 (s, 1H), 2.25 (s, 1H), 1.77-1.96 (m, 12H). ¹³C NMR (101 MHz, CDCl₃): δ 179.3, 154.1, 141.1, 139.0, 136.8, 134.9, 134.4, 117.5, 85.1, 63.3, 57.8, 39.3, 37.1, 32.7, 29.8, 28.3. MS (ESI⁻): m/z=425.1 [M−H]⁻.

### Synthesis of Compound 4

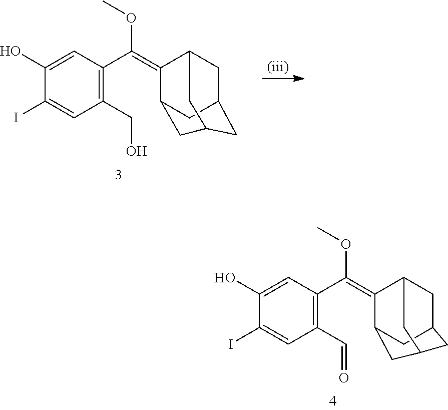 [SMILES: COC(=C1C2CC3CC(C2)CC1C3)c1cc(O)c(I)cc1CO ; "(iii)" ; COC(=C1C2CC3CC(C2)CC1C3)c1cc(O)c(I)cc1C=O]

To a solution of compound 3 (825.1 mg, 2 mmol), silica gel (1 g) and pyridine (403 μl, 5 mmol) in anhydrous dichloromethane (20 ml) was added dropwise pyridinium dichromate (1128.6 mg, 3 mmol) at 0° C. and stirred for 3 h. Upon completing the reaction, the silica gel solid was filtered and the filtrate was washed with saturated NH₄Cl (2×100 ml) and dichloromethane (2×100 ml). The organic layer was dried with anhydrous Na₂SO₄, and evaporated in vacuo which was further purified with silica gel chromatography using hexane/EtOAc to give a white solid compound 4 (577 mg, 68% yield).

¹H NMR (400 MHz, CDCl₃): δ 10.03 (s, 1H), 8.32 (s, 1H), 6.91 (s, 1H), 3.30 (s, 4H), 2.25 (s, 1H), 1.74-1.97 (m, 12H). ¹³C NMR (101 MHz, CDCl₃): δ 190.1, 159.1, 142.1, 138.5, 138.0, 135.6, 130.1, 117.1, 85.3, 57.6, 39.1, 39.1, 37.0, 32.8, 30.1, 28.2. MS (ESI⁻): m/z=424.1 [M−H]⁻.

### Synthesis of Compound 5

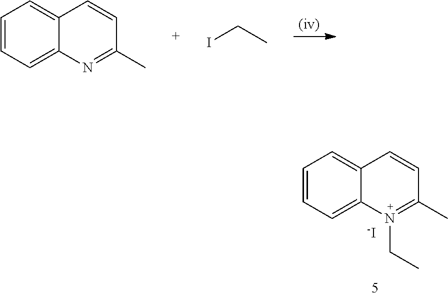

To a solution of 2-methylquinoline (1430 mg, 10 mmol) in ACN (2 ml) was added iodoethane (1206 μl, 15 mmol) at room temperature and stirred overnight at 80° C. under N₂ atmosphere. The reaction mixture was filtered to obtain yellow crude solid which was washed with cold diethyl ether (3×100 ml) to give compound 5 (2.42 g, 85% yield).

¹H NMR (400 MHz, CD₃OD): δ 9.01 (d, J=8.4 Hz, 1H), 8.58 (d, J=8.8 Hz, 1H), 8.37 (d, J=8.4 Hz, 1H), 7.99 (d, J=8.4 Hz, 1H), 5.09 q, J=7.3 Hz, 2H), 3.17 (s, 3H), 1.68 (t, J=7.4 Hz, 3H). ¹³C NMR (101 MHz, CD₃OD): δ 161.6, 147.3, 140.1, 136.8, 132.0, 130.4, 126.6, 119.7, 22.9, 13.9. MS (ESI⁺): m/z 172.1 [M−I]⁺.

### Synthesis of compound DBQP

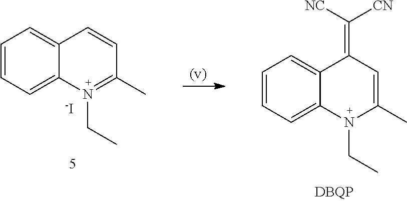

Sodium (92 mg, 4 mmol) was added dropwise to a solution of compound 5 (570 mg, 2 mmol) and malononitrile (528.2 mg, 8 mmol) in anhydrous ethanol (10 ml) and stirred for 10 h at an ice bath. The reaction mixture was concentrated in vacuo to remove ethanol to obtain the crude product. The residue was washed by DI water (3×30 ml) and EtOAc (3×30 ml) and dried at 50° C. in vacuo to give the pure DBQP (157 mg, 71%) as a yellow solid.

¹H NMR (400 MHz, CDCl₃): δ 9.00 (dd, J=1.6, 8.8 Hz, 1H), 7.72-7.76 (m, 1H), 7.62 (d, J=8.4 Hz, 1H), 7.37-7.41 (m, 1H), 6.74 (s, 1H), 4.35 (q, J=7.3 Hz, 2H), 2.58 (s, 3H), 1.48 (t, J=7.2 Hz, 3H). ¹³C NMR (101 MHz, CDCl₃): δ 153.6, 147.7, 138.2, 133.3, 126.9, 124.7, 121.6, 120.6, 119.1, 116.2, 110.0, 43.0, 21.6, 13.8. MS (ESI⁺: m/z 226.2 [M+H]⁺.

### Synthesis of Compounds DPA_{N} and IDPA_{N}

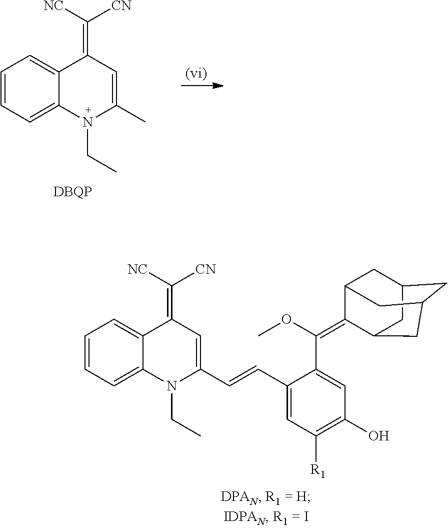

General method: the mixture of acceptor dicyanomethylene-4H derivative (0.06 mmol), piperidine (0.02 mmol), and phenol-adamantylidene derivative (compound 1 or compound 4) (0.05 mmol) were dissolved in anhydrous ACN (2.5 ml) and refluxed for 2-8 h under N₂ atmosphere. After completing reaction, the mixture was extracted with brine (2×20 ml) and dichloromethane (2×20 ml), and then the organic solution was collected and dried over anhydrous Na₂SO₄. The organic solvent was removed in vacuo to give the crude product. The crude product was further purified by HPLC using methanol and water as an eluent to afford corresponding products.

Compound DPA_{N} (24 mg, 92% yield) was obtained as a yellow solid.

¹H NMR (400 MHz, DMSO-d₆): δ 8.91 (d, J=8.4 Hz, 1H), 8.07 (d, J=9.2 Hz, 1H), 7.79-7.95 (m, 2H), 7.60 (d, J=8.4 Hz, 1H), 7.39 (dd, J=42.4, 16 Hz, 2H), 6.97 (s, 1H), 6.88 (dd, J=8.4, 2 Hz, 1H), 6.67 (d, J=6.4 Hz, 1H), 4.56 (q, J=5.4 Hz, 2H), 3.21 (s, 1H), 3.19 (s, 3H), 2.11 (s, 1H), 1.70-1.91 (m, 12H), 1.48 (t, J=7.0 Hz, 3H). ¹³C NMR (101 MHz, DMSO-d₆): δ 158.8, 152.2, 149.7, 140.6, 137.9, 137.7, 137.2, 133.7, 130.6, 128.5, 125.3, 125.1, 124.9, 120.6, 118.1, 117.6, 117.3, 116.0, 106.1, 56.3, 46.6, 43.7, 36.5, 32.2, 29.0, 27.6, 13.7. MS (ESI⁻): m/z 514.2 [M−H]⁻.

Compound IDPA_{N} (27.9 mg, 87% yield) was obtained as a yellow solid.

¹H NMR (400 MHz, DMSO-d₆): δ 10.92 (s, —OH, 1H), 8.92 (d, J=8.4 Hz, 1H), 8.43 (s, 1H), 8.09 (d, J=8.8 Hz, 1H), 7.93 (d, J=7.2 Hz, 1H), 7.61 (t, J=7.8 Hz, 1H), 7.46 (d, J=16 Hz, 1H), 7.31 (d, J=15.6 Hz, 1H), 6.94 (s, 1H), 6.77 (s, 1H), 4.59 (dd, J=14, 7.2 Hz, 1H), 3.18 (m, 3H), 2.12 (s, 1H), 1.63-1.95 (m, 12H), 1.38 (t, J=7.0 Hz, 3H). ¹³C NMR (101 MHz, DMSO-d₆): δ 157.8, 152.2, 149.6, 140.1, 137.9, 137.1, 136.9, 136.2, 133.7, 131.2, 127.3, 125.0, 120.7, 119.0, 118.2, 116.5, 106.4, 56.4, 46.8, 43.7, 36.4, 32.3, 31.3, 29.0, 13.7. MS (ESI⁻): m/z 640.3 [M−H]⁻.

### Synthesis of Compound 6

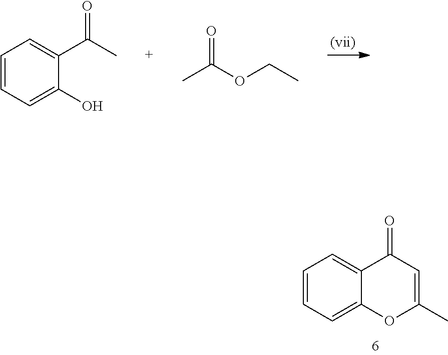

To a solution of 2-hydroxyacetophenone (10 g, 73 mmol) in anhydrous EtOAc (80 ml) was added dropwise sodium hydride (7 g, 184 mmol, 60% dispersion in mineral oil) at room temperature and stirred overnight at 80° C. Then, cold HCl (50 ml) (1 N) was added to quench the reaction and the organic layer was collected, dried with anhydrous Na₂SO₄ and evaporated in vacuo to obtain the crude diketone product. Several drops of concentrated HCl were added to a solution of the crude diketone in methanol (50 ml) and stirred for 14 h. The mixture was concentrated to remove methanol in vacuo, extracted with EtOAc (3×100 ml) and brine (3×100 ml). The organic layer was dried with anhydrous Na₂SO₄, evaporated in vacuo and purified with silica gel chromatography using hexane/EtOAc to give compound 6 as a white solid (7.59 g, 65% yield).

¹H NMR (CDCl₃, 400 MHz): δ 8.16 (d, J=7.6 Hz, 1H), 7.62 (t, J=7.8 Hz, 1H), 7.34-7.40 (m, 2H), 6.16 (s, 1H), 2.37 (s, 3H). ¹³C NMR (101 MHz, CDCl₃): δ 178.4, 166.3, 156.6, 133.6, 125.7, 125.0, 123.7, 117.9, 110.7, 20.7. MS (ESI⁺): m/z=161.1 [M+H]⁺.

### Synthesis of Compound DBP

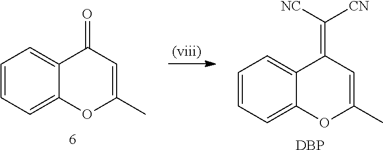

The mixture of compound 6 (4 g, 25 mmol) and malononitrile (3.3 g, 50 mmol) were dissolved in acetic anhydride (40 ml). The reaction mixture was stirred vigorously and refluxed for 6 h. After completion of the reaction, the mixture was concentrated in vacuo to remove acetic anhydride and extracted with saturated NaHCO₃ (3×200 ml) and EtOAc (3×200 ml). The organic layer was collected and dried with anhydrous Na₂SO₄ to obtain the residue which was purified with silica gel chromatography using hexane/EtOAc to give a brownish yellow solid (3.7 g, 71% yield).

¹H NMR (CDCl₃, 400 MHz): δ 8.90 (d, J=8.4 Hz, 1H), 7.69-7.75 (m, 1H), 7.42-7.48 (m, 2H), 6.71 (s, 1H), 2.44 (s, 3H). ¹³C NMR (101 MHz, CDCl₃): δ 178.4, 166.3, 156.6, 133.6, 125.7, 125.0, 123.7, 117.9, 110.7, 20.7. MS (ESI⁺): m/z=209.2 [M+H]⁺.

### Synthesis of Compounds DPA_{O} and IDPA_{O}

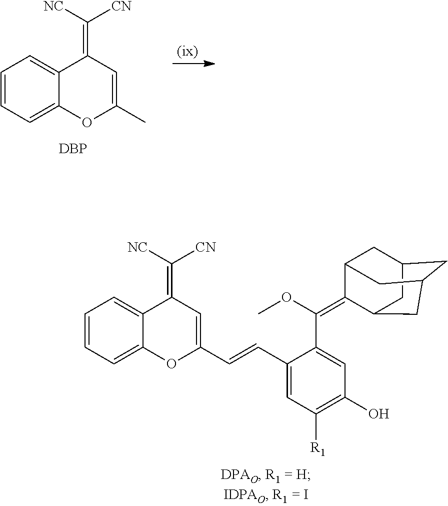

The reaction procedure is similar to the synthesis of DPA_{N} and IDPA_{N} above. The product yield was determined by HPLC analysis.

Compound DPA_{O} (19.8 mg, 81% yield) was obtained as a yellow solid.

¹H NMR (400 MHz, CDCl₃): δ 8.90 (d, J=8.0 Hz, 1H), 7.93 (d, J=16 Hz, 1H), 7.67-7.74 (m, 2H), 7.42-7.46 (m, 2H), 6.92 (d, J=7.6 Hz, 1H), 6.81 (s, 2H), 6.69 (d, J=16 Hz, 1H), 6.67 (d, J=6.4 Hz, 1H), 3.38 (s, 1H), 3.30 (s, 3H), 2.14 (s, 1H), 1.71-1.97 (m, 12H). ¹³C NMR (101 MHz, CDCl₃): δ 158.4, 158.0, 153.3, 152.5, 139.7, 138.8, 137.5, 134.8, 132.8, 127.8, 127.0, 126.9, 126.0, 125.9, 120.1, 118.6, 118.0, 117.9, 116.6, 116.4, 106.5, 57.2, 37.1, 32.8, 30.0, 28.4. MS (ESI⁻): m/z=487.2 [M−H]⁻.

Compound IDPA_{O} (20.6 mg, 67% yield) was obtained as an orange solid.

¹H NMR (400 MHz, DMSO-d₆): δ 8.72 (dd, J=8.4, 1.2 Hz, 1H), 8.39 (s, 1H), 7.89-7.91 (m, 1H), 7.7 (d, J=16 Hz, 1H), 7.59 (m, 1H), 7.49 (d, J=7.6 Hz, 1H), 7.44 (d, J=16 Hz, 1H), 6.99 (s, 1H), 6.78 (s, 1H), 3.21 (s, 3H), 1.64-2.02 (m, 13H). ¹³C NMR (101 MHz, DMSO-d₆): δ 158.4, 158.3, 152.8, 151.8, 139.4, 137.6, 136.7, 135.6, 134.8, 131.0, 127.2, 126.2, 124.8, 118.3, 117.6, 117.2, 116.2, 115.8, 106.5, 86.3, 60.0, 56.5, 36.4, 32.2, 29.2, 27.6. MS (ESI⁻): m/z=613.2 [M−H]⁻.

### Synthesis of Compound 7

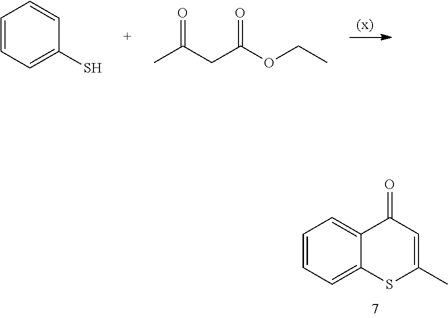

A mixture of benzenethiol (2.2 g, 20 mmol) and ethyl 3-oxobutanoate (2.86 g, 22 mmol) was added to polyphosphoric acid (24 ml) at 90° C. and stirred for 30 min. Then, the cooled mixture was quenched with ice water and extracted with EtOAc (3×50 ml) for three times. The organic solution was combined, dried over anhydrous Na₂SO₄, and evaporated under vacuum. The crude product was purified by silica gel chromatography using hexane/EtOAc as an eluent to give compound 7 as a yellow solid (2.39 g, 68% yield).

¹H NMR (400 MHz, CDCl₃): δ 8.49 (m, 1H), 7.52-7.59 (m, 2H), 7.47-7.51 (m, 1H), 6.83 (d, J=0.8 Hz, 1H), 2.45 (d, J=1.2 Hz, 3H). ¹³C NMR (101 MHz, CDCl₃): δ 180.7, 151.3, 137.8, 131.5, 130.9, 128.7, 127.6, 126.1, 125.0, 23.4. MS (ESI⁺): m/z=177.1 [M+H]⁺.

### Synthesis of Compound DBTP

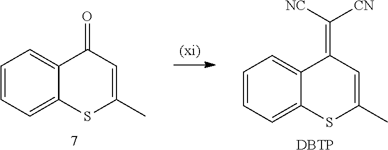

The mixture of compound 7 (500 mg, 2.84 mmol) and malononitrile (1.5 g, 22.7 mmol) in Ac₂O (10 ml) was heated to 140° C. and stirred for 2 h. After completing reaction, methanol (10 ml) was added and stirred at 50° C. for another 2 h. At last, the mixture solvent was evaporated under vacuum. The crude product was purified by silica gel chromatography using hexane/EtOAc as the eluent to give a yellow solid compound DBTP (337 mg, 53% yield).

¹H NMR (400 MHz, CDCl₃): δ 8.95 (d, J=8 Hz, 1H), 7.56-7.64 (m, 3H), 7.41 (s, 1H), 2.53 (s, 3H). ¹³C NMR (101 MHz, CDCl₃): δ 156.1, 149.0, 136.2, 131.9, 128.6, 128.5, 127.2, 125.1, 120.8, 117.1, 115.8, 23.6. MS (ESI⁺): m/z=225.1 [M+H]⁺.

### Synthesis of Compounds DPA_{Su} and IDPA_{Su}

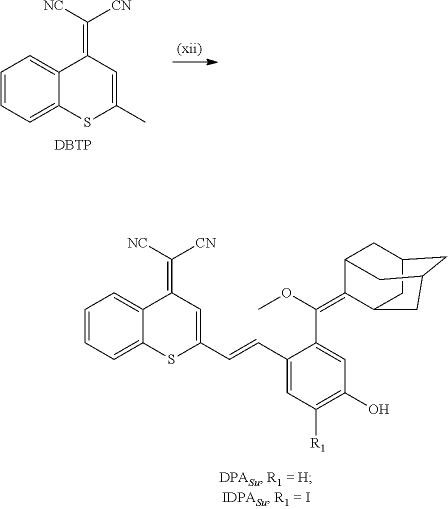

The reaction procedure is similar to the synthesis of DPA_{N} and IDPA_{N} above. The product yield was determined by HPLC analysis.

Compound DPA_{Su}(19.2 mg, 76%) was obtained as an orange solid.

¹H NMR (400 MHz, CDCl₃): δ 8.89 (d, J=8.4 Hz, 1H), 7.66 (d, J=8 Hz, 2H), 7.53-7.63 (m, 3H), 7.50 (s, 2H), 6.91-7.01 (m, 2H), 6.80 (s, 1H), 3.35 (s, 1H), 3.31 (s, 3H), 2.12 (s, 1H), 1.69-1.91 (m, 12H). ¹³C NMR (101 MHz, CDCl₃): δ 157.8, 156.2, 148.8, 139.8, 138.3, 136.0, 135.2, 133.1, 132.0, 128.4, 128.3, 127.7, 127.6, 127.1, 125.7, 123.4, 121.1, 118.0, 116.3, 67.5, 57.1, 39.2, 37.1, 33.5, 32.8, 30.0, 29.8, 29.8, 28.4. MS (ESI⁻): m/z=503.2 [M−H]⁻.

Compound IDPA_{Su} (19.8 mg, 67%) was obtained as an orange solid.

¹H NMR (400 MHz, CDCl₃): δ 10.99 (s, —OH, 1H), 8.73 (d, J=8.4 Hz, 1H), 8.42 (s, 1H), 7.97 (d, J=16 Hz, 1H), 7.76 (t, J=7 Hz, 1H), 7.67-7.71 (t, J=7 Hz, 1H), 6.91-7.01 (m, 2H), 7.63 (s, 1H), 7.36 (d, J=16.4 Hz, 1H), 6.75 (s, 1H), 3.28 (s, 1H), 3.31 (s, 3H), 2.01 (s, 1H), 1.60-1.81 (m, 12H). ¹³C NMR (101 MHz, CDCl₃): δ 158.0, 155.6, 148.2, 139.6, 137.1, 136.7, 134.1, 133.2, 132.6, 131.0, 128.4, 128.0, 127.6, 127.3, 124.6, 124.3, 120.9, 117.3, 116.3, 115.8, 86.2, 66.9, 56.4, 40.2, 39.9, 39.7, 39.5, 39.3, 39.1, 38.9, 38.5, 36.4, 32.2, 29.1, 27.6. MS (ESI⁻): m/z=629.1 [M−H]⁻.

### Synthesis of Compound 8

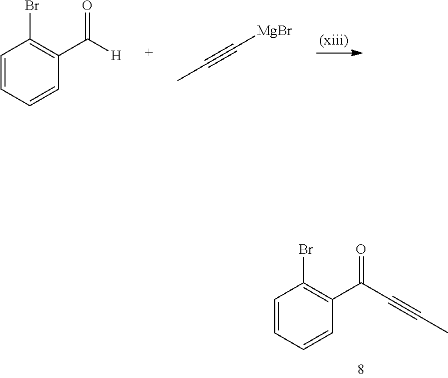

To a solution of propynylmagnesium bromide (36 ml, 0.5 M solution in THF, 18 mmol) was added o-bromobenzaldehyde (2.76 g, 15 mmol) in anhydrous THF (100 ml) at room temperature and stirred for 1 h. After completing the reaction, the reaction mixture was quenched with saturated aqueous NH₄Cl (3×50 ml), extracted with EtOAc (3×50 ml), and the organic layer was collected and dried over anhydrous Na₂SO₄. The solvent was then evaporated under vacuum to give the crude product which was further purified by silica gel chromatography to give a colorless liquid compound 8 (3.36 g, 100% yield).

¹H NMR (400 MHz, CDCl₃): δ 7.77 (d, J=1.6 Hz, 1H), 7.55 (dd, J=8, 1.6 Hz, 1H), 7.34-7.38 (m, 1H), 7.18 (m, 1H), 5.74-5.76 (m, 1H), 2.51 (d, J=1.2 Hz, 1H), 1.90 (d, J=1.2 Hz, 3H). ¹³C NMR (101 MHz, CDCl₃): δ 140.1, 133.1, 129.9, 128.6, 127.9, 122.8, 83.6, 78.2, 77.5, 77.2, 76.8, 64.4, 3.9. MS (ESI⁺): m/z=225.2 [M+H]⁺.

### Synthesis of Compound 9

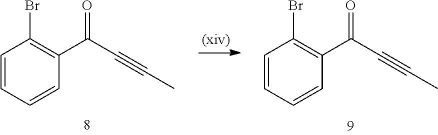

To a solution of compound 8 (2.24 g, 10 mmol) in DMSO (20 ml), 2-iodoxybenzoic acid (IBX) (3.36 g, 12 mmol) was added slowly at room temperature and stirred for 2 h. The reaction was quenched with water (20 ml) and extracted with EtOAc (20 ml) and brine (30 ml) for three times. The organic layer was combined and dried over anhydrous Na₂SO₄, filtered and concentrated under vacuum. The crude product was purified by column chromatography with hexane/EA to afford a colorless liquid compound 9 (2.13 g, 96% yield).

¹H NMR (400 MHz, CDCl₃): δ 7.97 (dd, J=7.6, 1.6 Hz, 1H), 7.61 (d, J=8 Hz, 1H), 7.37 (t, J=6.8 Hz, 1H), 7.30 (m, 1H), 2.08 (s, 3H). ¹³C NMR (101 MHz, CDCl₃): δ 177.5, 137.1, 134.8, 133.2, 133.0, 127.3, 120.9, 93.9, 79.9, 4.4. MS (ESI⁺): m/z=223.0 [M+H]⁺.

### Synthesis of Compound 10

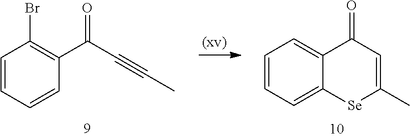

Powder Se (474 mg, 6 mmol) was added to a solution of NaBH₄ (189 mg, 5 mmol) in anhydrous dimethylformamide (DMF, 5 ml) at room temperature. The mixture was heated to 105° C. and stirred for 1 h. Then the solution of compound 9 (1.11 g, 5 mmol) in dry DMF (8 ml) was added dropwise to the mixture and stirred at 105° C. for another 4 h. After the reaction was cooled to room temperature, the mixture was extracted with EtOAc (20 ml) for three times. The combined organic layers were washed with brine and dried over anhydrous Na₂SO₄, filtered, and evaporated. The crude product was purified by column chromatography with hexane/EtOAc as the eluent to produce a yellow solid compound 10 (896 mg, 85% yield).

¹H NMR (400 MHz, CDCl₃): δ 8.62-8.64 (d, J=8.1 Hz, 1H), 7.64-7.68 (m, 1H), 7.52-7.59 (m, 2H), 6.98 (d, J=1.2 Hz, 1H), 2.59 (d, J=0.8 Hz, 1H). ¹³C NMR (101 MHz, CDCl₃): δ 182.5, 152.9, 136.8, 131.5, 130.1, 128.2, 127.7, 127.0, 77.5, 77.2, 76.8, 25.1. MS (ESI⁺): m/z=224.1 [M+H]⁺.

### Synthesis of Compound DBSeP

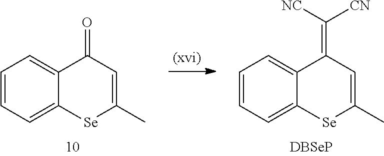

To a solution of compound 10 (500 mg, 2.23 mmol) in Ac₂O (10 ml) was added malononitrile (1.18 g, 17.8 mmol) and stirred for 2 h at 145° C. After finishing the reaction, methanol (10 ml) was added and stirred for 2 h when the reaction was cooled down to the room temperature. The solvent was then evaporated by vacuum evaporation. The crude product was purified by silica gel chromatography using hexane/EtOAc as the eluent to give a brown solid DBSeP (188 mg, 31% yield).

¹H NMR (400 MHz, CDCl₃): δ 8.72-8.76 (m, 1H), 7.68-7.73 (m, 1H), 7.50-7.56 (m, 3H), 2.60 (d, J=0.8 Hz, 3H). ¹³C NMR (101 MHz, CDCl₃): δ 159.0, 151.3, 135.5, 131.7, 129.9, 129.2, 128.3, 126.2, 122.5, 116.7, 115.4, 25.4. MS (ESI⁺): m/z=273.0 [M+H]⁺.

### Synthesis of Compounds DPA_{Se} and IDPA_{Se}

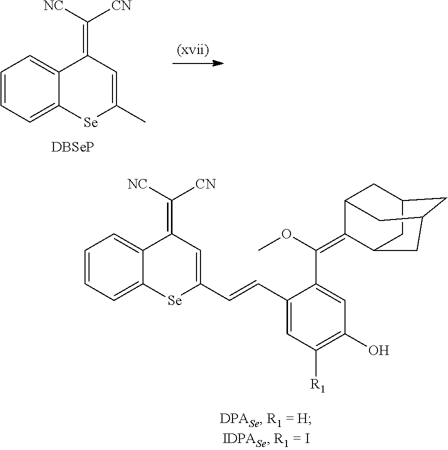

The reaction procedure is similar to the synthesis of DPA_{N} and IDPA_{N} above. The product yield was determined by HPLC analysis.

Compound DPA_{Se} (14 mg, 52% yield) was obtained as an orange solid.

¹H NMR (400 MHz, CDCl₃): δ 8.71 (m, 1H), 7.73 (d, J=3.6 Hz, 1H), 7.73 (d, J=8.4 Hz, 2H), 7.63 (s, 1H), 7.51-7.53 (m, 2H), 7.43 (d, J=16 Hz, 1H), 7.08 (d, J=16 Hz, 1H), 6.89 (d, J=8.4 Hz, 1H), 6.76 (s, 1H), 3.35 (s, 1H), 3.29 (s, 3H), 2.11 (s, 1H), 1.69-2.01 (m, 12H). ¹³C NMR (101 MHz, CDCl₃): δ 159.1, 157.3, 150.6, 140.0, 138.4, 136.8, 134.1, 132.8, 131.8, 129.7, 129.6, 128.2, 127.6, 127.2, 125.6, 123.2, 118.0, 116.3, 57.1, 39.2, 37.2, 32.8, 30.0, 28.4. MS (ESI⁻): m/z 551.1 [M−H]⁻.

Compound IDPA_{Se} (19.8 mg, 38% yield) was obtained as an orange solid.

¹H NMR (400 MHz, DMSO-d₆): δ 10.99 (s, —OH, 1H), 8.58 (dd, J=7.6, 1.2 Hz, 1H), 8.42 (s, 1H), 8.12 (dd, J=8, 1.6 Hz, 1H), 7.73-7.67 (m, 2H), 7.62-7.67 (m, 2H), 7.21 (d, J=16 Hz, 1H), 6.74 (s, 1H), 3.27 (s, 1H), 3.20 (s, 3H), 1.58-2.0 (m, 13H). ¹³C NMR (101 MHz, DMSO-d₆): δ 158.7, 158.0, 150.6, 139.7, 137.0, 136.7, 134.1, 133.2, 132.2, 131.0, 130.2, 129.2, 128.2, 127.5, 126.4, 126.1, 122.9, 117.0, 116.3, 115.5, 86.2, 71.2, 56.4, 36.4, 32.2, 29.1, 27.6. MS (ESI⁻): m/z=677.0 [M−H]⁻.

### Synthesis of Compounds alkyne-PEG2000-OMe and alkyne-PEG2000-COOH

The compounds alkyne-PEG2000-OMe and alkyne-PEG2000-COOH were successfully synthesized according to previous literature (He, S. et al., J. Am. Chem. Soc. 2020, 142, 7075-7082).

Alkyne-PEG2000-OMe was obtained as a white powder.

¹H NMR (400 MHz, CDCl₃): δ 4.20 (d, J=2.4 Hz, 2H), 3.64-3.69 (m, 176H), 3.54 (dd, J=2.8 5.2 Hz, 4H), 3.37 (s, 3H), 2.43 (t, J=2.4 Hz, 1H). MALDI-TOF-MS: 1500-2500 Da. Alkyne-PEG2000-COOH as a white solid was obtained. ¹H NMR (400 MHz, CDCl₃): δ 3.96 (s, 2H), 3.44-3.82 (m, 182H), 2.43 (t, J=2.2 Hz, 1H). MALDI-TOF-MS: 1400-2600 Da.

Alkyne-PEG2000-cRGD as a white powder was also obtained.

A mixture of N-hydroxysuccinimide (17.2 mg, 0.15 mmol), 4-dimethylaminopyridine (18.4 mg, 0.15 mmol), and 1-ethyl-3-(3-dimethylaminopropyl)carbodiimide (23.2 mg, 0.15 mmol) was added to a solution of alkyne-PEG2000-COOH (200 mg, 0.1 mmol) in anhydrous dichloromethane (5 ml) and stirred for 12 h at room temperature. After monitoring the reaction by TLC analysis, the mixture was extracted with saturated NaCl (3×20 ml) and dichloromethane (3×20 ml). The organic layer was combined, dried in anhydrous Na2SO4 and concentrated in vacuo. The solution of the obtained residue in anhydrous DMF (2 ml) was added to a mixture of cRGD (94.8 mg, 0.1 mmol) and NaHCO₃ (16.8 mg, 0.1 mmol) in anhydrous DMF (3 ml) and stirred overnight at room temperature. After completing reaction, the mixture was dialyzed against DI water (spectra/pore, MWCO 2 KDa) and then freeze-dried to give alkyne-PEG2000-cRGD (~248 mg, ~80% cRGD grafting yield) as a white solid.

### Synthesis of Compound 11

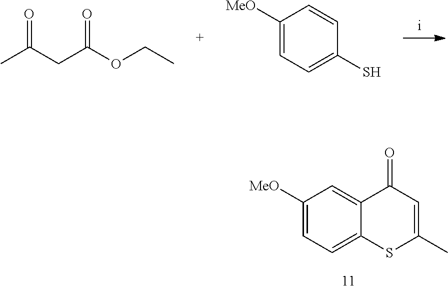

This reaction procedure is similar to the synthesis of compound 7 above. Briefly, 4-methoxybenzenethiol (2.8 g, 20 mmol) and ethyl 3-oxobutanoate (2.86 g, 22 mmol) were added to polyphosphoric acid (24 ml) at 90° C. and stirred for 30 min. Then, the mixture was cooled to room temperature and then worked up. The residue was purified by silica gel chromatography using hexane/EtOAc as an eluent to give compound 11 as a yellow solid (2.8 g, 68% yield).

¹H NMR (400 MHz, CDCl₃): δ 7.94 (d, J=2.8 Hz, 1H), 7.45 (d, J=8.8 Hz, 1H), 7.20 (dd, J=8.8, 2.8 Hz, 1H), 6.84 (s, 1H), 3.91 (s, 3H), 2.46 (s, 3H). ¹³C NMR (101 MHz, CDCl₃): δ 180.4, 159.5, 151.3, 132.2, 129.8, 127.4, 124.2, 122.0, 109.0, 55.8, 23.3. MS (ESI⁺): m/z=207.3 [M+H]⁺.

### Synthesis of Compound 12

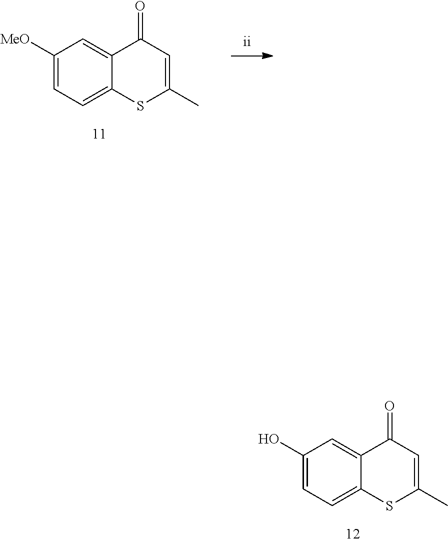

To a solution of compound 11 (1030 mg, 5 mmol) in anhydrous dichloromethane (10 ml) was added dropwise BBr₃ (949 ml, 10 mmol) at 0° C. and stirred for 6 h. The grey solid was precipitated and washed with EtOAc (3×50 ml) to give pure compound 12 (912 mg, 95%) as a pale yellow solid.

¹H NMR (400 MHz, DMSO-d₆): δ 10.16 (s, 1H), 7.68 (d, J=2.8 Hz, 1H), 7.64 (d, J=8.8 Hz, 1H), 7.18 (dd, J=8.8, 2.8 Hz, 1H), 6.77 (s, 1H), 2.44 (s, 3H). ¹³C NMR (101 MHz, DMSO-d₆): δ 178.9, 157.3, 151.5, 131.5, 128.1, 126.8, 123.2, 121.4, 111.8, 40.2, 39.9, 39.7, 39.5, 39.3, 39.1, 38.9, 22.7. MS (ESI⁻): m/z=191.2 [M−H]⁻.

### Synthesis of Compound 13

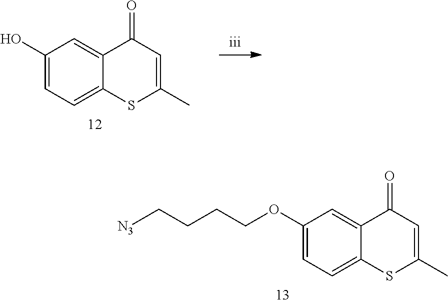 [SMILES: Cc1cc(=O)c2cc(O)ccc2s1 ; Cc1cc(=O)c2cc(OCCCCN=[N+]=[N-])ccc2s1]

The mixture of compound 12 (480 mg, 2.5 mmol), K₂CO₃ (691 mg, 5 mmol) and 1-azido-4-iodobutane (675 mg, 3 mmol) in anhydrous DMF (5 ml) was stirred overnight at 80° C. under N₂ atmosphere. The mixture was poured into saturated NH₄Cl solution (50 ml) and extracted with EtOAc (3×100 ml). The organic layers were combined and concentrated in vacuo. Then, the residue was purified with silica gel chromatography using hexane/EtOAc as an eluent to give compound 13 as a yellow solid (2.8 g, 87% yield).

¹H NMR (400 MHz, CDCl₃): δ 7.92 (d, J=2.4 Hz, 1H), 7.47 (d, J=8.8 Hz, 1H), 7.20 (dd, J=8.8, 2.4 Hz, 1H), 6.84 (s, 1H), 4.11 (t, J=6.0 Hz, 2H), 3.37 (t, J=6.8 Hz, 2H), 2.46 (s, 3H), 1.88-1.95 (m, 2H), 1.77-1.84 (m, 2H). ¹³C NMR (101 MHz, CDCl₃): δ 158.8, 151.4, 129.8, 127.5, 124.2, 122.2, 109.8, 77.5, 77.2, 76.8, 67.8, 51.3, 26.5, 25.9, 23.4. MS (ESI⁺): m/z=312.1 [M+Na]⁺.

### Synthesis of Compound 14

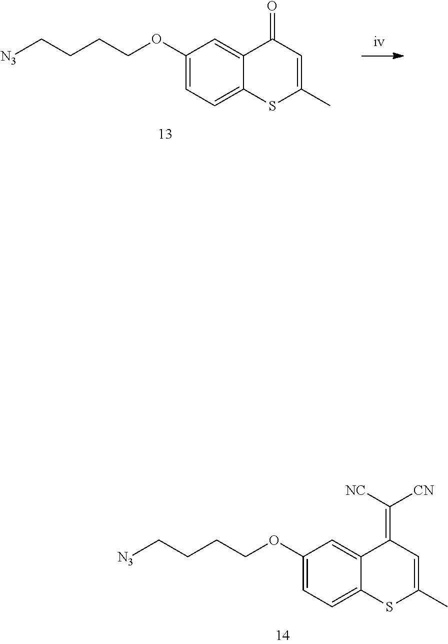 [SMILES: Cc1cc(=O)c2cc(OCCCCN=[N+]=[N-])ccc2s1 ; CC1=CC(=C(C#N)C#N)c2cc(OCCCCN=[N+]=[N-])ccc2S1]

The reaction procedure of compound 14 is similar to the synthesis of compound 8 above. Compound 14 (303.4 mg, 45% yield) as a yellow solid was obtained by silica gel chromatography using hexane/EtOAc as the eluent.

¹H NMR (400 MHz, CDCl₃): δ 8.44 (s, 1H), 7.56 (d, J=8.4 Hz, 1H), 7.42 (s, 1H), 7.28 (s, 1H), 4.15 (t, J=5.0 Hz, 2H), 3.39 (t, J=6.0 Hz, 2H), 2.55 (s, 3H), 1.93-1.96 (m, 2H), 1.79-1.86 (m, 2H). ¹³C NMR (101 MHz, CDCl₃): δ 158.8, 155.8, 149.0, 128.2, 126.3, 122.8, 120.4, 117.7, 110.3, 68.3, 51.2, 26.4, 25.8, 23.5. (ESI⁺): m/z=338.2 [M+H]⁺.

### Synthesis of Compound 15

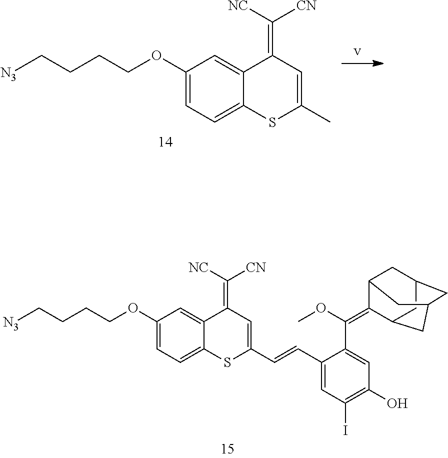 [SMILES: CC1=CC(=C(C#N)C#N)c2cc(OCCCCN=[N+]=[N-])ccc2S1 ; COC(=C1C2CC3CC(C2)CC1C3)c1cc(O)c(I)cc1/C=C/C1=CC(=C(C#N)C#N)c2cc(OCCCCN=[N+]=[N-])ccc2S1]

To a solution of compound 14 (67.4 mg, 0.2 mmol) and compound 4 (127.2 mg, 0.3 mmol) in anhydrous I (4 ml) was added piperidine (20 ml, ~0.2 mmol) under N₂ atmosphere and stirred at 90° C. for 2 h. The product compound 15 (136.7 mg, 92% yield) as an orange solid was purified by HPLC using ACN/water as eluent.

¹H NMR (400 MHz, CDCl₃): δ 8.40 (d, J=2.4 Hz, 1H), 8.08 (s, 1H), 7.58 (d, J=8.8 Hz, 1H), 7.55 (s, 1H), 7.45 (d, J=16.4 Hz, 1H), 7.24 (d, J=2.4 Hz, 1H), 7.00 (d, J=16.4 Hz, 1H), 6.90 (s, 1H), 4.14 (t, J=6.0 Hz, 2H), 3.38 (t, J=2.4 Hz, 2H), 3.34 (s, 1H), 3.29 (s, 3H), 2.13 (s, 1H), 1.88-2.01 (m, 12H), 1.68-1.76 (m, 4H). ¹³C NMR (101 MHz, CDCl3): δ 158.8, 156.0, 155.8, 147.9, 139.5, 138.7, 136.0, 133.7, 133.6, 129.5, 128.7, 127.0, 124.7, 122.9, 121.5, 117.4, 110.4, 86.3, 68.3, 57.3, 51.3, 39.2, 37.1, 32.9, 31.6, 30.4, 30.0, 29.8, 28.4, 26.4, 25.8. (ESI⁻): m/z 742.2 [M−H]⁻.

### Synthesis of Compound Uncaged MRAP

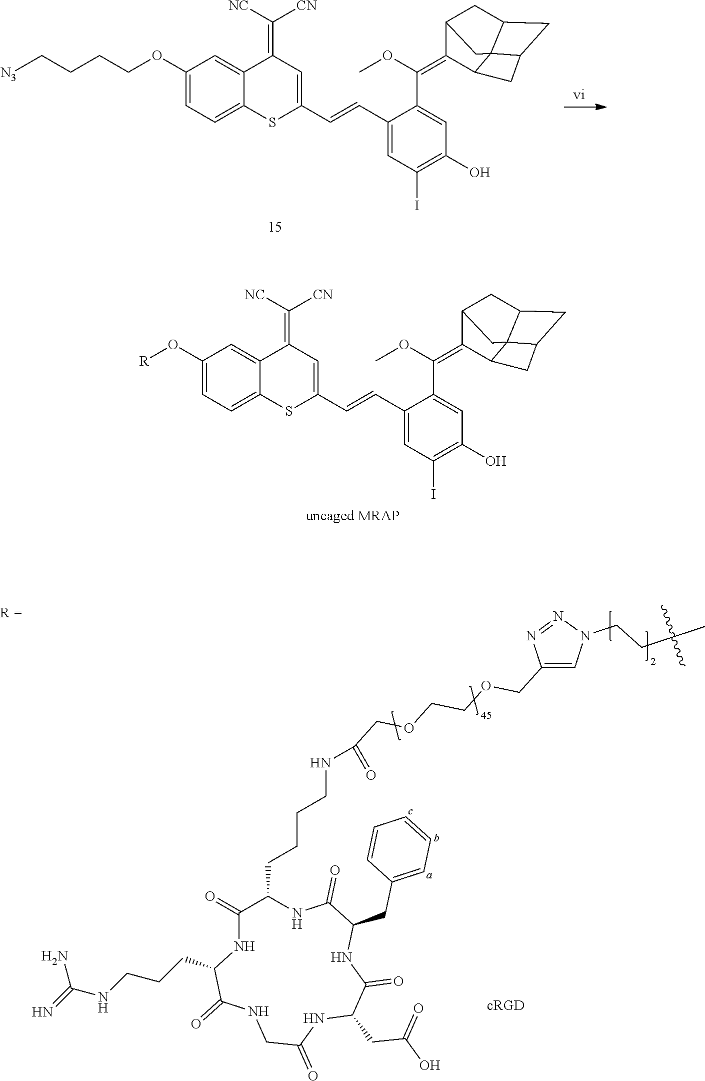

The mixture of CuSO₄·5H₂0 (0.62 mg, 0.0025 mmol) and ascorbic acid sodium (2.47 mg, 0.0125 mmol) in DI water (0.4 ml) was added to a solution of compound 15 (37.2 mg, 0.05 mmol) and alkyne-PEG2000-cRGD (124.1 mg, 0.05 mmol, ~80% cRGD grafting yield) in DMSO (4 ml) under N₂ atmosphere and stirred at room temperature for 4 h. After completing the reaction, the mixture was dialyzed against DI water (spectra/pore, MWCO 2 KDa) and then freeze-dried to give uncaged MRAP (153 mg, 95% product yield after click reaction and ~80% cRGD grafting yield) as an orange powder.

¹H NMR (400 MHz, DMSO-d₆): δ 8.42 (s, 1H), 8.10 (s, 1H), 7.92 (d, J=8.8 Hz, 1H), 7.66 (d, J=7.2 Hz, 1H), 7.61 (s, 1H), 7.41-7.45 (m, 2H), 7.35 (d, J=8.4 Hz, 1H), 7.18-7.26 (m, 4H), 6.73 (s, 1H), 4.51 (s, 1.6H), 4.41-4.44 (m, 2H), 4.43-4.44 (m, 1H), 4.13-4.14 (m, 4H), 3.85-3.87 (m, 1.6H), 3.86 (d, J=7.6 Hz, 2H), 3.40-3.68 (m, 180H), 3.25-3.28 (m, 3H+3.2H), 3.20 (s, 3H), 2.94-3.09 (m, 4H), 2.67-2.80 (m, 1.6H), 1.78-2.01 (m, 13H), 1.59-1.74 (m, 8H).

### Synthesis of Compound Z-Val-Cit

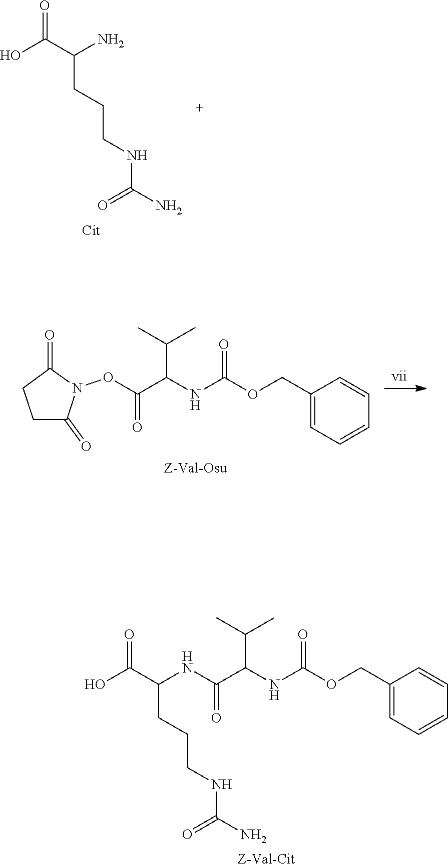 [SMILES: NC(=O)NCCCC(N)C(=O)O ; CC(C)C(NC(=O)OCc1ccccc1)C(=O)ON1C(=O)CCC1=O ; CC(C)C(NC(=O)OCc1ccccc1)C(=O)NC(CCCNC(N)=O)C(=O)O]

To a solution of Z-Val-Osu (1.74 g, 5 mmol) in dimethoxyethane (15 ml) was added a solution of L-Citrulline (Cit, 1.31 g, 7.5 mmol) and NaHCO₃ (630 mg, 7.5 mmol) in DI water (10 ml) and stirred for 30 min at 0° C. Then, the mixture was stirred vigorously for another 16 h at room temperature. Water (20 ml, containing 2 ml of saturated NaHCO₃) was added, and the mixture was then washed with EtOAc (50 ml×3) and then acidified to pH 3 with 1N HCl. The resulting suspension was extracted with 10% n-butanol/EtOAc (50 ml×3). The combined organic layers were dried with anhydrous Na₂SO₄ and concentrated in vacuo to give a white solid (3.39 g, 96% yield).

¹H NMR (400 MHz, DMSO-d₆): δ 7.28-7.38 (m, 5H), 5.03 (s, 2H), 4.14 (d, J=13.2 Hz, 5.2 Hz, 1H), 3.92 (d, J=8.8 Hz, 7.2 Hz, 1H), 2.95 (t, J=6.4 Hz, 1H), 1.92-2.00 (m, 1H), 1.66-1.74 (m, 1H), 1.55-1.61 (m, 1H), 1.35-1.41 (m, 2H), 0.88 (d, J=6.8 Hz, 3H), 0.83 (d, J=6.8 Hz, 3H). ¹³C NMR (101 MHz, DMSO-d₆): δ 173.6, 173.0, 171.5, 159.0, 156.2, 137.2, 128.5, 127.9, 127.8, 65.3, 60.0, 52.00, 30.6, 28.5, 26.7, 25.3, 19.3, 18.2. (ESI⁺): m/z=409.3 [M+H]⁺.

### Synthesis of Compound 16

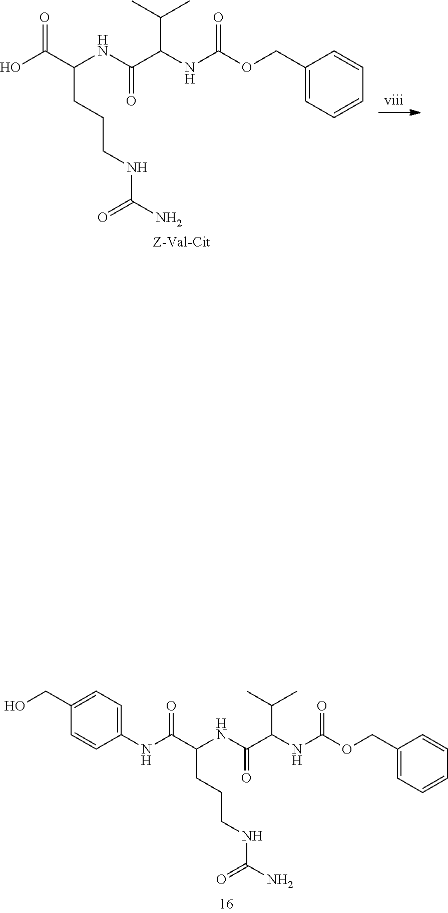 [SMILES: CC(C)C(NC(=O)OCc1ccccc1)C(=O)NC(CCCNC(N)=O)C(=O)O ; CC(C)C(NC(=O)OCc1ccccc1)C(=O)NC(CCCNC(N)=O)C(=O)Nc1ccc(CO)cc1]

To a solution of Z-Val-Cit (816.4 mg, 2 mmol) and N-ethoxycarbonyl-2-ethoxy-1,2-dihydroquinoline (741.9 mg, 3 mmol) in THF (10 ml) was added a solution of 4-aminobenzyl alcohol (369.5 mg, 3 mmol) in THF (5 ml) at room temperature and stirred for 8 h at 0° C. After finishing the reaction, the solvent was removed in vacuo to obtain the residue, which was then washed with ice diethyl ether (25 ml×3) and EtOAc (25 ml×3) to give the pure product 16 (923.9 mg, 90% yield) as a white solid.

¹H NMR (400 MHz, DMSO-d₆): δ 8.07 (d, J=7.6 Hz, 1H), 7.54 (d, J=8.4 Hz, 1H), 7.30-7.37 (m, 5H), 7.24 (d, J=8.4 Hz, 1H), 5.04 (s, 1H), 4.43 (s, 3H), 3.92 (t, J=7.6 Hz, 1H), 2.89-3.05 (m, 1H), 1.94-2.01 (m, 1H), 1.55-1.74 (m, 2H), 1.31-1.48 (m, 1H), 0.88 (d, J=6.4 Hz, 3H), 0.84 (d, J=6.8 Hz, 3H). ¹³C NMR (101 MHz, DMSO-d₆): δ 171.2, 170.4, 158.9, 156.2, 137.5, 137.5, 137.1, 128.3, 127.8, 127.6, 126.9, 118.9, 65.4, 62.6, 60.1, 53.0, 30.4, 29.5, 26.7, 19.2, 18.1. (ESI⁺): m/z=514.4 [M+H]⁺.

### Synthesis of Compound 17

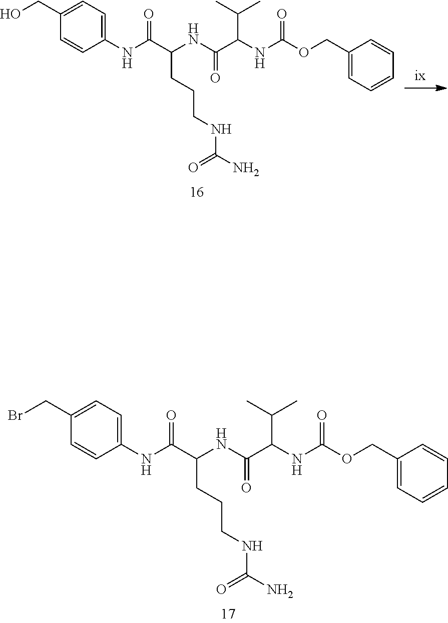 [SMILES: CC(C)C(NC(=O)OCc1ccccc1)C(=O)NC(CCCNC(N)=O)C(=O)Nc1ccc(CO)cc1 ; CC(C)C(NC(=O)OCc1ccccc1)C(=O)NC(CCCNC(N)=O)C(=O)Nc1ccc(CBr)cc1]

To a solution of compound 16 (256.6 mg, 0.5 mmol) in THF (10 ml) was added PBr₃ (189.8 μl, 2.0 mmol) and stirred for 6 h at 0° C. After finishing the reaction, the reaction was extracted with saturated NaHCO₃ (50 ml×3) and EtOAc (50 ml×3). The combined organic layers were dried with anhydrous Na₂SO₄ and concentrated in vacuo to give a white solid without purification.

### Synthesis of Compound 18

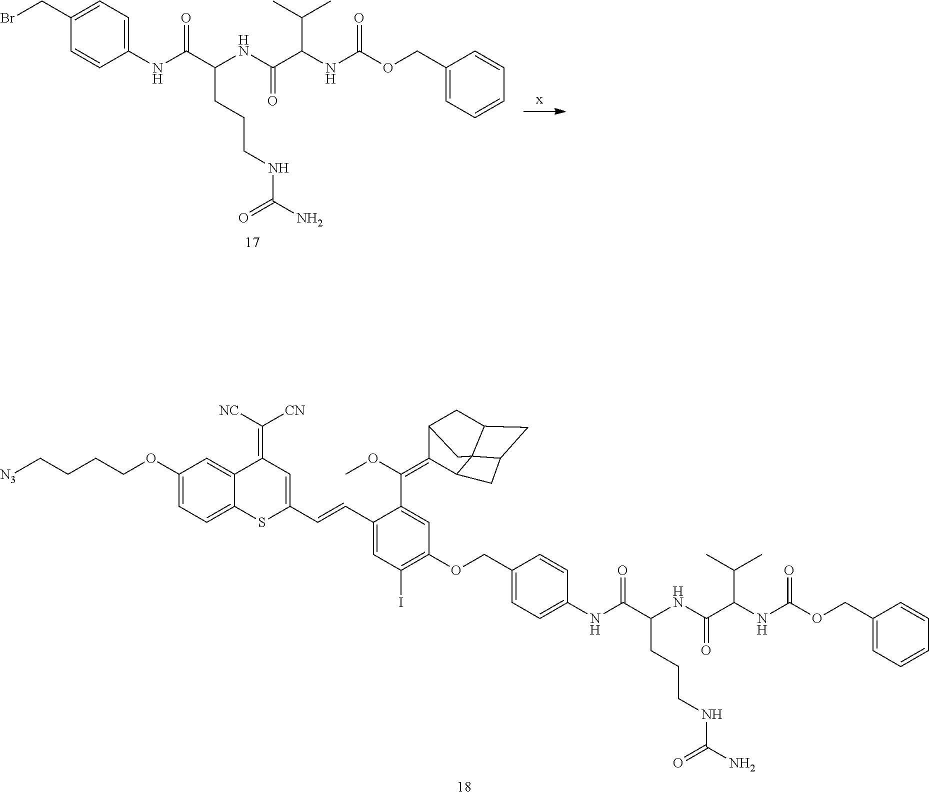 [SMILES: CC(C)C(NC(=O)OCc1ccccc1)C(=O)NC(CCCNC(N)=O)C(=O)Nc1ccc(CBr)cc1 ; CO/C(=C1\C2CC3CC4CC1C34C2)c1cc(OCc2ccc(NC(=O)C(CCCNC(N)=O)NC(=O)C(NC(=O)OCc3ccccc3)C(C)C)cc2)c(I)cc1/C=C/C1=CC(=C(C#N)C#N)c2cc(OCCCCN=[N+]=[N-])ccc2S1]

To a solution of compound 17 (0.5 mmol) and Cs₂CO₃ (162.9 mg, 0.5 mmol) in THF/ACN (2 ml/2 ml, V/V) was added compound 15 (185.8 mg, 0.25 mmol) and stirred for 4 h at 0° C. After monitoring the reaction by HPLC, the residue was then purified by HPLC to obtain pure product 18 (207.4 mg, 67% yield) as an orange solid.

¹H NMR (400 MHz, DMSO-d₆): δ 8.50 (s, 1H), 8.28 (d, J=2 Hz, 1H), 8.10 (d, J=7.6 Hz, 1H), 7.93 (d, J=8.8 Hz, 1H), 7.75 (d, J=16.4 Hz, 1H), 7.61-7.64 (m, 3H), 7.41-7.43 (m, 2H), 7.34-7.38 (m, 6H), 6.89 (s, 1H), 5.24 (s, 2H), 5.04 (s, 2H), 4.43 (dd, J=7.6 Hz, 7.2 Hz, 1H), 3.92-3.95 (m, 1H), 3.28 (s, 1H), 3.21 (s, 3H), 2.95-3.04 (m, 2H), 1.78-2.01 (m, 13H), 1.68-1.72 (m, 5H), 1.61 (m, 4H), 0.88 (d, J=6.8 Hz, 3H), 0.84 (d, J=6.8 Hz, 3H). ¹³C NMR (101 MHz, DMSO-d6): δ 171.1, 170.5, 158.0, 157.4, 156.1, 155.1, 148.2, 139.4, 138.6, 137.0, 136.3, 132.4, 130.9, 129.4, 128.8, 128.2, 128.1, 127.7, 127.5, 125.8, 125.7, 125.2, 122.0, 120.7, 118.9, 117.6, 116.0, 114.8, 110.9, 88.4, 70.2, 67.7, 65.9, 65.3, 60.0, 56.4, 53.0, 50.2, 36.4, 32.1, 30.3, 29.4, 29.1, 28.9, 28.6, 27.5, 26.7, 25.6, 24.9, 22.0, 19.1, 18.1, 13.9. (ESI⁺): m/z=1239.2 [M+H]⁺.

### Synthesis of Compounds cRGD-Free MRAP and MRAP

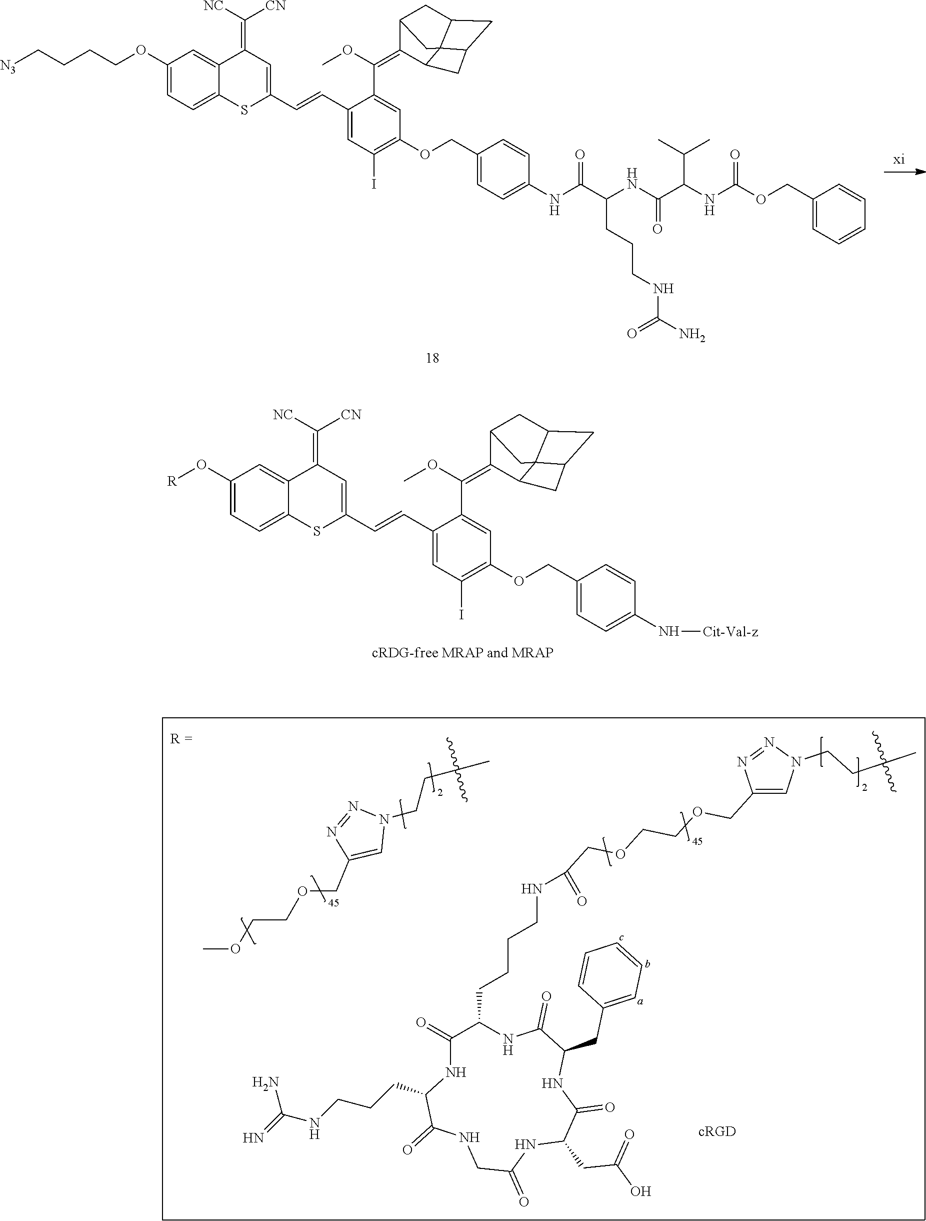

To a solution of compound 18 (24.8 mg, 0.02 mmol) and alkyne-PEG2000-cRGD or alkyne-PEG2000-OMe (0.02 mmol) in DMSO (4 ml) was added a solution of CuSO₄·5H₂0 (0.25 mg, 0.001 mmol) and ascorbic acid sodium (0.99 mg, 0.005 mmol) in DI water (0.4 ml) under N₂ atmosphere and stirred at room temperature for 4 h. After completing the reaction, the mixture was dialyzed against DI water (spectra/pore, MWCO 2.5 KDa) and then freeze-dried to give corresponding compounds. cRGD-free MRAP (64.7 mg, ~100% product yield) was obtained as an orange powder.

¹H NMR (400 MHz, DMSO-d₆): δ 8.50 (s, 1H), 8.27 (d, J=2.4 Hz, 1H), 8.10 (d, J=3.2 Hz, 1H), 7.93 (d, J=8.8 Hz, 1H), 7.75 (d, J=16.4 Hz, 1H), 7.61-7.64 (m, 3H), 7.44-7.56 (m, 2H), 7.40 (s, 1H), 7.33 (m, 6H), 6.89 (s, 1H), 5.41 (s, 2H), 5.04 (s, 2H), 4.41-4.46 (m, 1H), 4.14 (d, J=2.0 Hz, 1H), 4.08-4.11 (m, 2H), 3.90-3.95 (m, 2H), 3.51-3.54 (m, 176H), 3.41-3.44 (m, 4H), 3.34 (s, 3H), 3.24 (s, 3H), 3.20 (s, 1H), 1.77-2.02 (m, 13H), 1.60-1.73 (m, 9H), 0.83-0.88 (m, 6H).

MRAP (74.4 mg, ~100% product yield after click reaction and ~80% cRGD grafting yield) was obtained as an orange powder.

¹H NMR (400 MHz, DMSO-d₆): δ 8.50 (s, 1H), 8.27 (d, J=1.6 Hz, 1H), 8.10 (d, J=2.0 Hz, 1H), 7.92 (d, J=8.8 Hz, 1H), 7.74 (d, J=16 Hz, 1H), 7.61-7.63 (m, 3H), 7.40-7.45 (m, 2H), 7.40 (s, 1H), 7.34-7.37 (m, 6H), 7.17-7.24 (m, 4H), 6.89 (s, 1H), 5.42 (s, 2H), 5.03 (s, 2H), 4.51 (s, 1.6H), 4.43-4.44 (m, 1H), 4.13-4.14 (m, 4H), 3.93-3.95 (m, 1.6H), 3.88 (d, J=1.6 Hz, 2H), 3.90-3.95 (m, 2H), 3.51-3.69 (m, 176H), 3.39-3.42 (m, 4H), 3.34 (s, 3H), 3.25-3.28 (m, 3H+3.2H), 3.20 (s, 3H), 2.94-3.09 (m, 4H), 2.67-2.80 (m, 1.6H), 1.77-2.07 (m, 13H), 1.49-1.71 (m, 9H+8H), 0.83-0.88 (m, 6H).

### Example 2. Optical Properties of DPAs and IDPAs

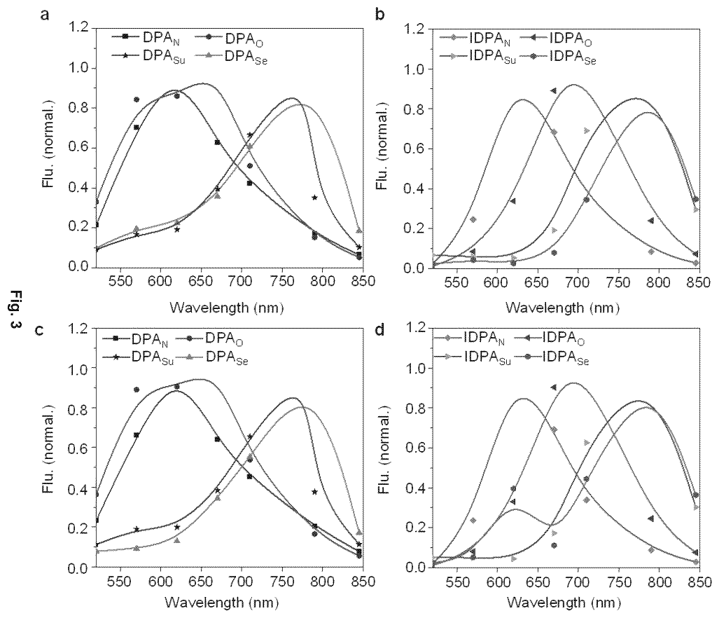

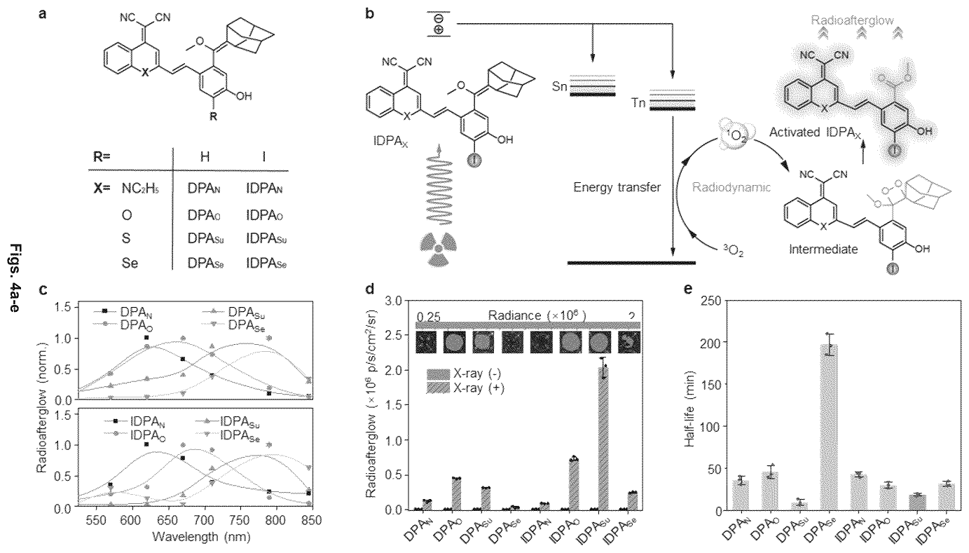

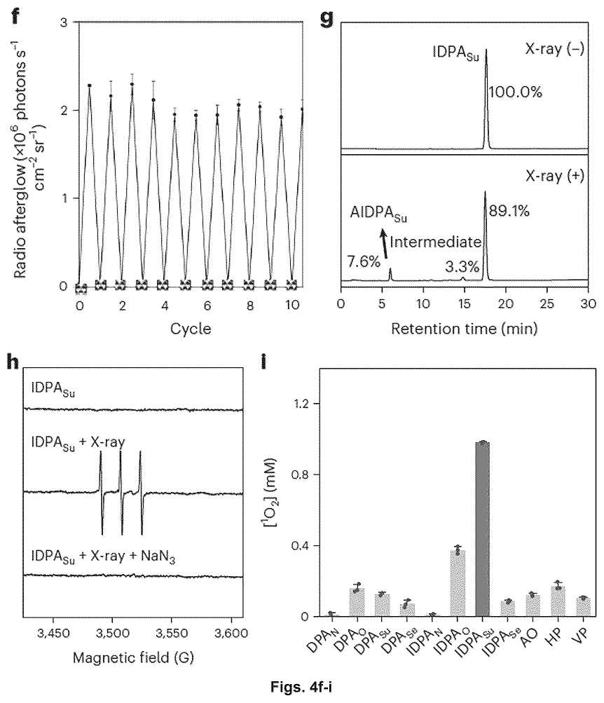

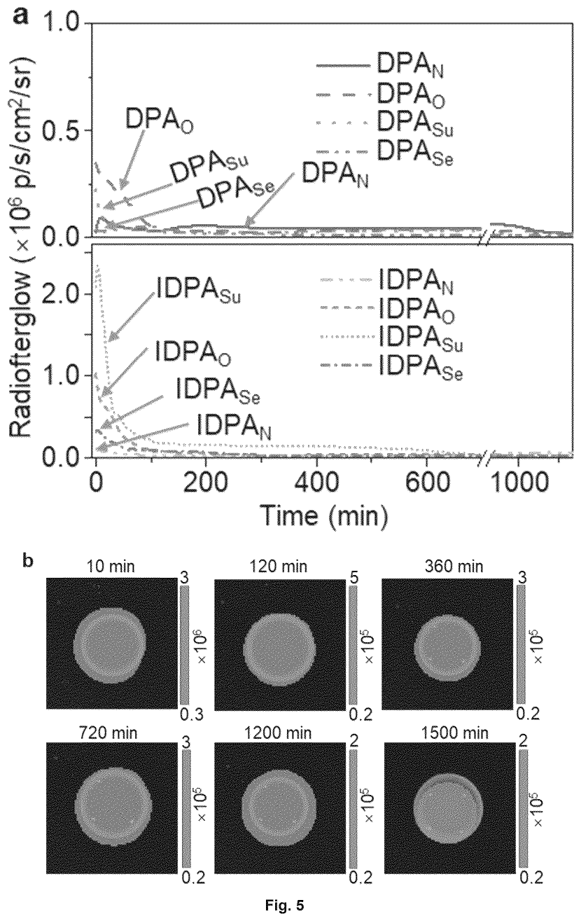

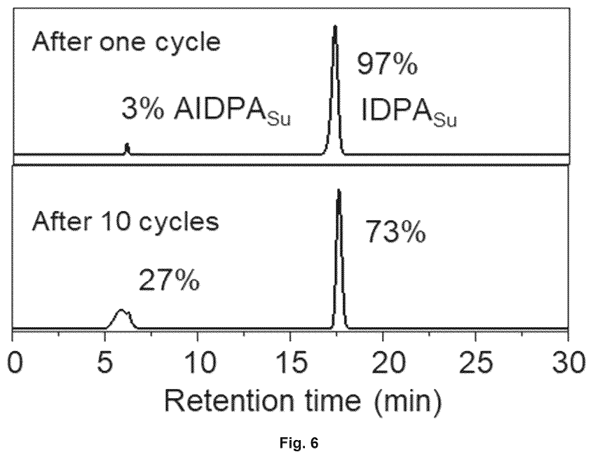

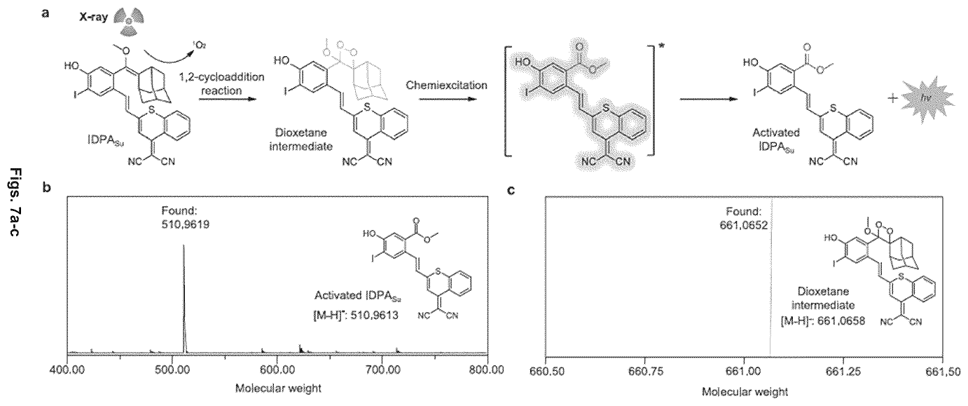

The optical properties of DPAs and IDPAs were investigated in an aqueous solution (FIGS. 2 and 3). After X-ray irradiation (250 μGy s⁻¹) for 20 s, DPAs and IDPAs showed radio afterglow with emission maxima ranging from 624 to 792 nm (FIG. 4c), with the spectral profiles close to their fluorescence (FIG. 3). The presence of iodine did not change the emission maximum; however, it increased the radio afterglow intensities (~1.62-fold for IDPA_{O} versus DPA_{O}, 6.46-fold for IDPA_{Su} versus DPA_{Su}, 6.65-fold for IDPA_{Se} versus DPA_{Se}) (FIG. 4d). Their half-lives ranged from 9.2 to 196.8 min (FIG. 4e). Among them, IDPA_{Su} had the highest brightness and relatively longer emission maximum at ~773 nm, with a long-lasting radio afterglow up to ~25 h (FIG. 5). Meanwhile, the radio afterglow of IDPA_{Su} could be repetitively induced with minimal intensity decrease after ten cycles (FIG. 4f) as one cycle of irradiation consumed only ~3% of IDPA_{Su} (FIG. 6), showing its ability for longitudinal imaging.

### Example 3. Radio Afterglow Mechanism of Organic Luminophores

To study the radio afterglow mechanism, the sample of IDPA_{Su} after X-ray irradiation was analysed with HPLC and high-resolution mass spectrometry.

### ESR Study of ROS Generation

To study ¹O₂ generation, a mixture of DPAS or IDPAS (100 M) and TEMP (10 mM) in PBS (0.01 M, pH 7.4, 50% DMSO) was irradiated by X-rays at 2 Gy (100 mGy s⁻¹ for 20 s) or 5 mGy (250 μGy s⁻¹ for 20 s). The ESR signals of the mixture were recorded with a Bruker A300 instrument. The ESR signal of the free radical 2,2,6,6-tetramethyl-1-piperidinyloxyl (g=2.0060) was generated by the reaction of ¹O₂ with TEMP. The ¹O₂ quencher NaN₃ (1 mM) was added to validate the afterglow mechanism of IDPA_{Su}. Additionally, other commercially available radio sensitizers (such as acridine orange, hematoporphyrin and verteporfin) were measured and compared. The procedure of block depths was similar to that of the tissue penetration depth study and the ¹O₂ generation for different depth tissues was measured. The ESR signal changes of MRAP in the absence or presence of CatB were measured in a similar way. The ESR signals of 2,2,6,6-tetramethyl-1-piperidinyloxyl from the reaction of ¹O₂ with TEMP were plotted as a function of the concentration of ¹O₂, which was generated by the addition of NaOCl to H₂O₂. This generated the standard curve between the ESR intensity and ¹O₂ concentration.

To study other ROS generation, fluorescence sensors DHR123 (for O₂·⁻ detection) and HPF (for ·OH detection) were used. For O₂·⁻ detection, DHR123 (10 PM) was added to a solution of IDPA_{Su} (10 M) in PBS (0.01 M pH 7.4, 50% DMSO), followed by X-ray irradiation at different doses of 5 mGy (250 μGy s⁻¹ for 20 s) and 2 Gy (100 mGy s¹ for 20 s). The fluorescence changes in DHR123 were recorded at λₑₓ of 505 nm and λₑₘ of 534 nm. For ·OH detection, HPF (10 M) was added in a solution of IDPA_{Su} (10 M) in PBS (0.01 M, pH 7.4, 50% DMF), followed by X-ray irradiation at different doses, namely, 5 mGy (250 μGy s⁻¹ for 20 s) and 2 Gy (100 mGy s⁻¹ for 20 s). The fluorescence changes in HPF were collected at ex of 450 nm and λₑₘ of 515 nm. The ROS production was calculated by the corresponding standard curve.

### Results and Discussion

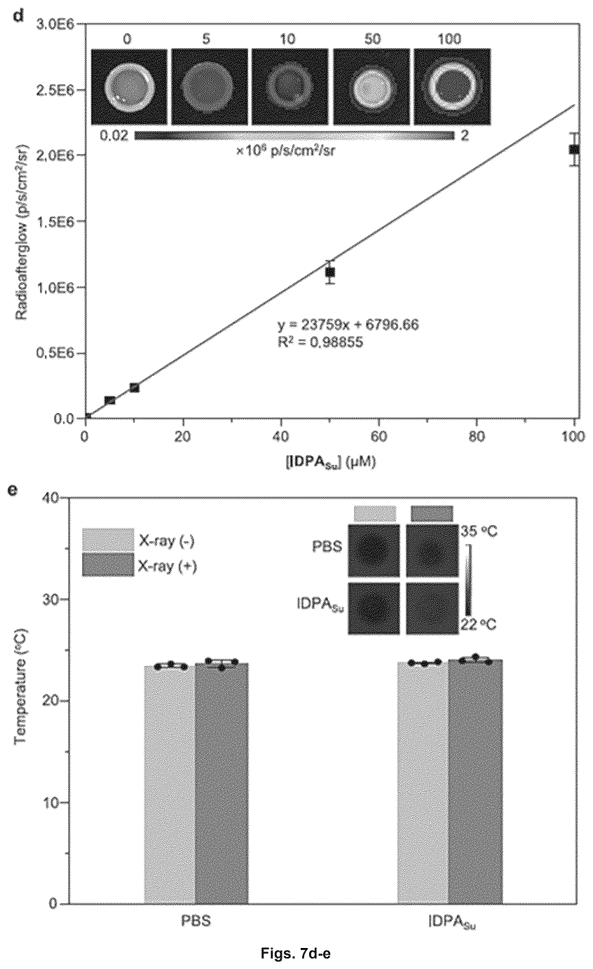

The HPLC profile of X-ray-irradiated IDPA_{Su} (AIDPA_{Su}) (FIG. 4g) showed two new elution peaks at 6.0 and 14.8 min with a conversion yield of 7.6% and 3.3%, respectively. The high-resolution mass spectrometry analysis revealed that the products peaking at 6.0 and 14.8 min had a mass of 510.9619 and 661.0652, which were assigned to activated IDPA_{Su} and a dioxetane compound, respectively (FIGS. 7b-c).

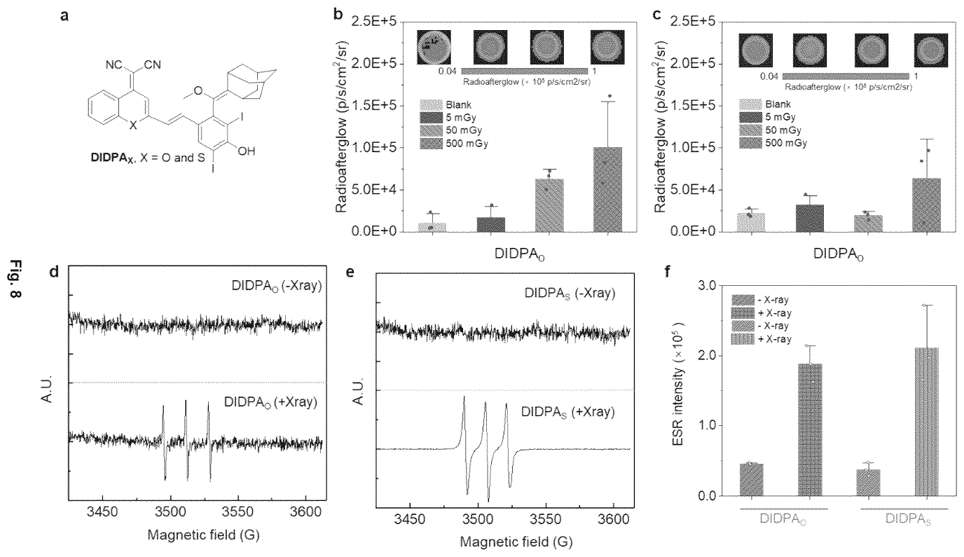

To explore the influence of iodine atom on radio afterglow and radiodynamic activity, the DIDPA_{X} (X=O and S) with two iodine substituent groups were first synthesized. The radio afterglow intensities of DIDPA_{O} and DIDPA_{S} were extremely low even after increasing X-ray dosage as two iodine atoms made very low quantum yield (FIGS. 8a-c). However, the radiodynamic activities showed a slightly increase (FIGS. 8d-f). For example, X-ray triggered ¹O₂ generation from DIDPA_{S} displayed ~1.2-fold higher than that of IDPAs.

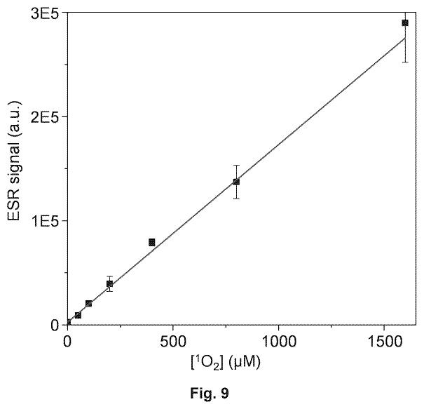

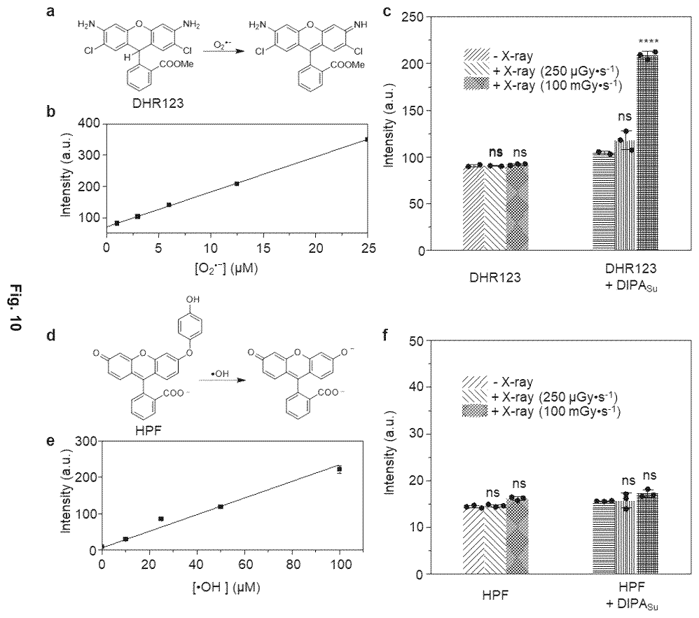

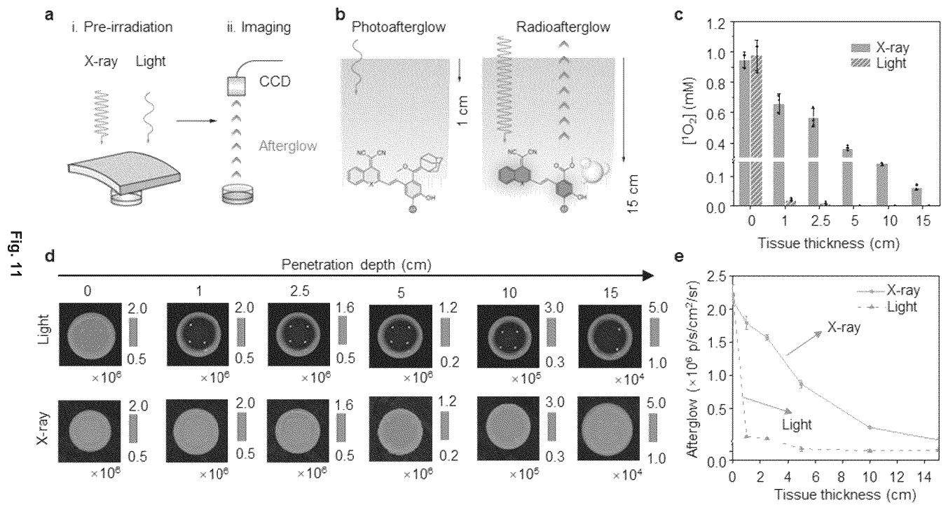

The formation of the dioxetane intermediate indicates that ROS were generated from X-ray irradiation. This was confirmed by ESR spectroscopy, showing the generation of ¹O₂ by DPA_{Su} and IDPA_{Su} under X-ray irradiation (FIG. 4h). Among them, the generation of ¹O₂ by IDPA_{Su} was the highest (FIGS. 4i and 9); moreover, other ROS were undetectable and only minimal O₂·⁻ could be generated at elevated radiation dosage (FIG. 10).

TABLE 1
The linear regression equation in FIG. 9.

| Equation | y = a + b*x |
|---|---|
| Plot | B |
| Weight | Instrumental (=1/ei\hat{ }2) |
| Intercept | 2540.51385 ± 74.00884 |
| Slope | 170.55695 ± 11.25282 |
| Residual Sum of Squares | 20.41467 |
| Pearson's r | 0.98929 |
| R-Square (COD) | 0.9787 |
| Adj. R-Square | 0.97444 |

These data reveal that the radio afterglow mechanism of DPAs and IDPAs is closely associated with the radiodynamic process (FIG. 4b). First, the X-ray irradiation of DPA or IDPA leads to the radiodynamic generation of ¹O₂. Then, the in situ-generated ¹O₂ reacts with DPA or IDPA via a cycloaddition reaction and converts it into a dioxetane intermediate. At last, this intermediate gradually decomposes into the corresponding activated products (activated DPA or IDPA) and releases photons. Although both luminophores generated ¹O₂ under X-ray irradiation, the presence of heavy iodine in IDPAs can not only enhance their X-ray absorption but also efficiently populate triplet excitons. Thus, IDPAs radiodynamically produced more ¹O₂ than the iodine-free counterparts, which facilitated the formation of the dioxetane intermediate and consequently emitted stronger radio afterglow. Note that the radiodynamic ¹O₂ generation of IDPA_{Su} was at least 5.7 times higher than the commercially available radio sensitizers including acridine orange (AO), hematoporphyrin (HP) and verteporfin (VP), showing its high promise for RDT.

### Example 4. Deep-Tissue Radio Afterglow Dynamic Performance

The tissue penetration of radio afterglow by IDPA_{Su} was compared with photoafterglow.

### Screening for Radio Afterglow Molecules

DPAs or IDPAs (100 μM) in 0.01 M PBS (pH 7.4, 50% DMSO) were irradiated by X-rays at 250 μGy s⁻¹ for 20 s (total, 5 mGy) under the specific setup parameter of 10 kV and 75 μA. Then, the radio afterglow signal was collected with an IVIS imaging system under the bioluminescence mode with an open filter and an acquisition time of 60 s. Finally, the radio afterglow intensities were quantified by a region-of-interest analysis using Living Image 4.3 software. In addition, the radio afterglow spectra of the candidates were acquired under the same test conditions with specific emission filters, and time courses of the signal were recorded as a function of time.

### Tissue Penetration Depth Study

IDPA_{Su} (100 M, 100 l) solution in a round cap with a diameter of 1 cm was prepared and placed under the chicken breast tissue with different thicknesses (0, 1.0, 2.5, 5.0, 10.0 and 15.0 cm). To avoid the influence of afterglow intensity differences on tissue penetration, the X-ray source (Moxtek, Inc.) and xenon lamp (PLS-SEX300D, Beijing Perfectlight Technology Co., Ltd.) were optimized to ensure that all the samples at 0 cm (without coverage) received the same dose of X-rays (5 mGy, 250 μGy s⁻¹ for 20 s) or light (180 mW cm⁻² for 20 s) and thus the same afterglow intensity. After the cessation of irradiation, radio afterglow or photoafterglow was detected with or without the chicken breast tissues using the IVIS bioluminescence mode with an open filter and acquisition time of 120 s.

### Measurement of Limit of Detection (LOD)

To test the LOD of IDPA_{Su} for in vivo RAI, 20 μl of the mixture solution of IDPA_{Su} at different concentrations (100.0, 50.0, 20.0, 10.0, 5.0, 1.0, 0.5 and 0 μM) and solubilizing agent (2-carboxyethyl)-o-cyclodextrin sodium salt (β-CD) at 100 μM was subcutaneously injected into the left flank of BALB/c nude mice (six weeks old). After 30 min, the left flank of mice were irradiated with X-rays at 5 mGy (250 μGy s⁻¹ for 20 s) as the other areas were protected by a 3.4-mm-thick lead shield with a notch under the X-ray irradiation. Radio afterglow signals were recorded. The detection limit was calculated using this equation: LOD=3σ/k, where a represents the s.d. of blank and k implies the slope of the radio afterglow intensities.

To study the LOD of MRAP for CatB detection, MRAP (100 μM) was incubated in PBS (0.01 M, pH 7.4), EDTA (1 mM) and glutathione (5 mM) with different concentrations of CatB (0, 0.10, 0.25, 0.50, 1.00 and 5.00 μg ml⁻¹) for 6 h, followed by X-ray irradiation (5 mGy, 250 Gy s⁻¹ for 20 s). After irradiation, the signals were immediately recorded. The LOD was determined as LOD=3σ/k.

### Confocal Microscopy

Confocal microscopy images were acquired on a Nikon A1 confocal laser scanning microscope (Nikon Instruments). The tissue sections were examined using Leica RM2016 (Leica Inc.) and an ECLIPSE 80i microscope (Nikon Instruments).

### Histological Analysis

For H&E staining, tumours and organs were fixed with 4% paraformaldehyde, dehydrated in the graded ethanol solution (100%, 90% and 75%), embedded in paraffin and cut into sections with a thickness of 5 m. The sections were incubated with haematoxylin for ~3-5 min and eosin for 5 min, followed by washing with distilled water. Then, the sections were hydrated and sealed for observation under microscopes. For immunofluorescence staining, the tumours were fixed with 4% paraformaldehyde, dehydrated using 30% sucrose aqueous solution, followed by embedding in frozen optimal cutting temperature medium. Then, the entire tissue block was frozen by liquid nitrogen and was transferred to a cryotome cryostat (-20° C., Leica Biosystems). The block was cut into the desired section with a thickness of 10 m and the sections were placed on glass slides, followed by drying at 25° C. for 60 min. The sections were washed thrice with 0.1% Triton X-100 in PBS (0.01 M, pH 7.4) and incubated with 3.0% bovine serum albumin for 1 h at 25° C. Then, the sections were washed with PBS (0.01 M, pH

7.4) and incubated with the corresponding antibodies (anti-cleaved caspase-3 antibody and anti-caspase-1 antibody) for 1 h, followed by washing thrice with PBS. The sections were counterstained with Alexa Fluor 594 secondary antibody for 1 h at 25° C. DAPI was used to stain the cell nuclei, and the sections were observed by a confocal laser scanning microscope. TUNEL or anti-cleaved caspase-3 staining was conducted according to the protocol (Abcam company). For SOSG staining, subcutaneous U87-tumour-bearing mice were intratumorally injected with PBS (20 μl) and IDPA_{Su} (0.1 μmol kg⁻¹, solubilized with 0.1 μmol kg⁻¹ of β-CD). After 30 min post-injection, the tumour was locally injected with an SOSG sensor (20 μl, 100 μM) and subsequently treated with or without X-ray irradiation (0.1 Gy, 5 mGy s⁻¹ for 20 s). After that, the mice were euthanized to collect the tumour, which was fixed with 4% paraformaldehyde, and cut into sections (10 μm). These tumour sections were also stained with DAPI and then observed using the confocal laser scanning microscope. The fluorescence intensity of SOSG in each picture was quantified by ImageJ v1.51a software. The procedure for SOSG staining in the orthotopic U87 tumour model was similar to that in the subcutaneous U87 tumour model.

### Heat Generation Study

IDPA_{Su} (100 μM, 100 μL) solution and PBS in a round cap with the diameter of 1 cm were prepared and then irradiated by X-ray at dose of 5 mGy (250 μGy·s⁻¹ for 20 s), respectively. The temperatures of the IDPA_{Su} solution and PBS were recorded by FUR Infrared Camera (FLIR A35-Series, FUR Systems).

### Results and Discussion

To induce afterglow, IDPA_{Su} was irradiated with X-ray or light through chicken breast tissues before signal detection (FIGS. 11a-b). To eliminate the influence of intensity difference on tissue penetration, X-ray and light irradiation were optimized to ensure similar intensity without tissue coverage. At the tissue depth of 1 cm, photoafterglow was hard to induce due to light scattering and reflection, showing the intensity (7.20×10⁴ photons s⁻¹ cm⁻² sr⁻¹) slightly higher than the background (~6.10×10³ photons s⁻¹ cm⁻² sr⁻¹); in contrast, radio afterglow reached ~1.79×10⁶ photons s⁻¹ cm⁻² sr⁻¹ (FIG. 11e). Radio afterglow of IDPA_{Su} was inducible and detectable even through ~15 cm tissue, showing ~4.2-fold higher signal than that of IDPA_{Su} without X-ray irradiation and tissue (FIG. 11d).

The deep-tissue radiodynamic process of IDPA_{Su} was tested by measuring ¹O₂ generation at different tissue depths and compared with photosensitization (FIG. 11c). Without tissue coverage, both X-ray and light effectively induced ¹O₂ generation from IDPA_{Su}. At the tissue depth of 1 cm, the concentration of ¹O₂ generated by the X-ray irradiation of IDPA_{Su} slightly decreased by ~30%; in contrast, it was close to the background for light irradiation. Furthermore, the radiodynamic generation of ¹O₂ for IDPA_{Su} was still inducible at a tissue depth of 15 cm.

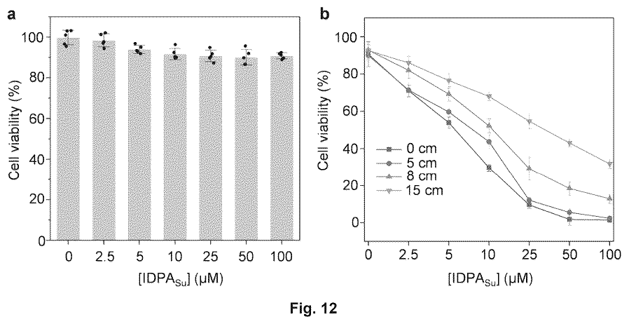

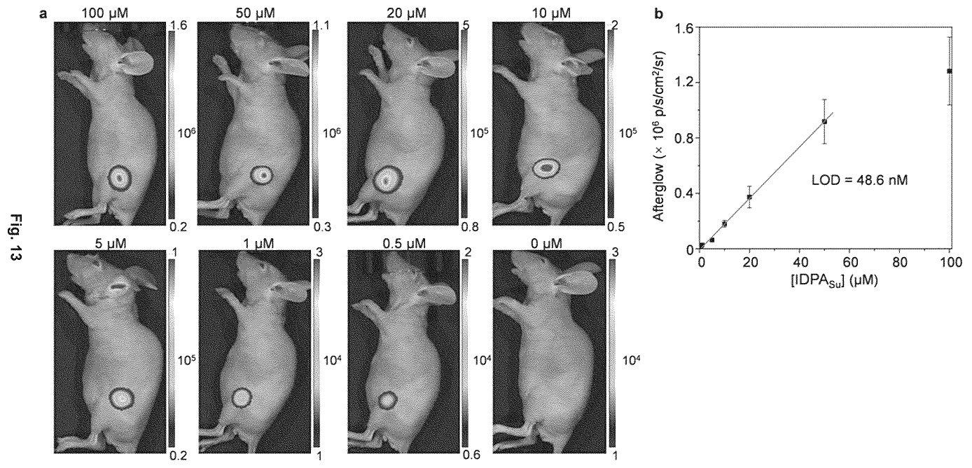

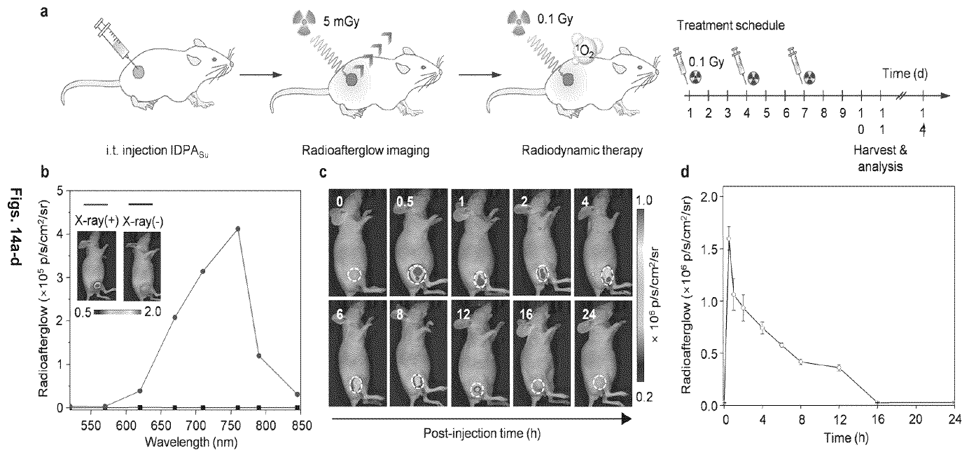

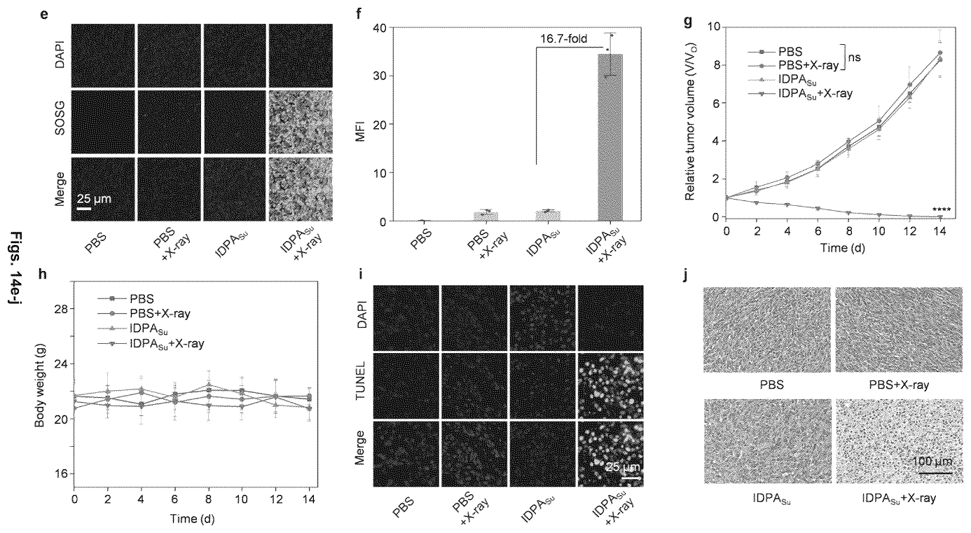

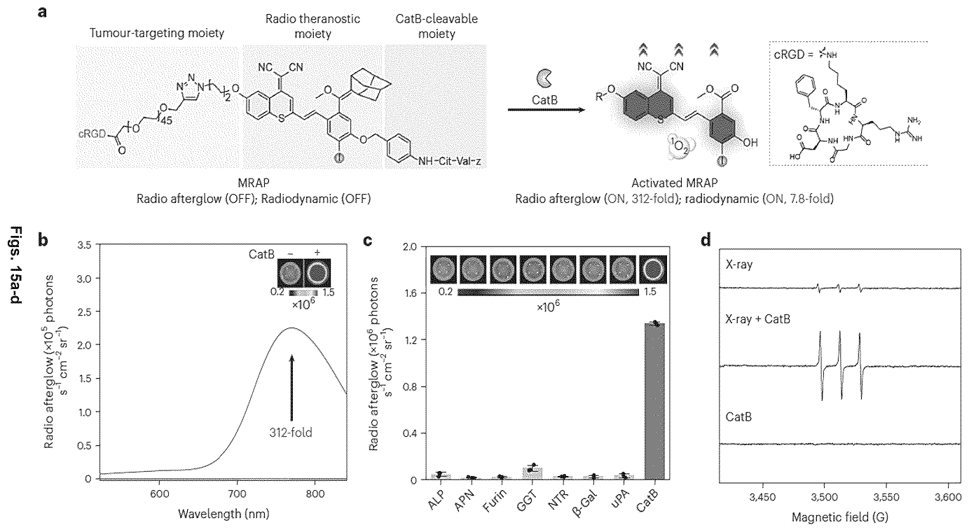

To validate the suitability of IDPA_{Su} for cancer radio theranostics, the cell viabilities with and without X-ray irradiation were studied (FIG. 12). IDPA_{Su} itself exhibited 91% viability even at a high concentration of 100 μM. However, under X-ray irradiation (2 Gy, 100 mGy s⁻¹ for 20 s), the viability drastically dropped to 1.4% due to the production of cytotoxic ¹O₂. Such a radio therapeutic effect was still inducible even at the tissue depth of 15 cm (31.9%). The radio afterglow signals of the IDPA_{Su} solution had a linear correlation with its concentrations, and radiation did not cause any heat generation even at a concentration of 100 μM of IDPA_{Su} (FIG. 7e). The in vivo imaging sensitivity of IDPA_{Su} was studied by detecting the radio afterglow of subcutaneously injected IDPA_{Su}, showing a LOD of IDPA_{Su} as low as 49 nM (FIG. 13). The RDT ability was validated by the intratumoral injection of IDPA_{Su} into the tumour-bearing nude mice followed by X-ray irradiation with a total dosage of 0.3 Gy in three fractions (see Example 6 for more details). This IDPA_{Su}-mediated RDT completely inhibited the tumour growth and caused no loss in body weight (FIG. 14). These data confirm that IDPA_{Su} is promising for cancer theranostics.

TABLE 2
The linear regression equation in FIG. 13b.

| Equation | y = a + b*x |
|---|---|
| Plot | B |
| Weight | No Weighting |
| Intercept | 651.04114 ± 5981.9 |
| Slope | 18319.0083 ± 307.5 |
| Residual Sum of Sq | 1.18251E9 |
| Pearson's r | 0.99916 |
| R-Square (COD) | 0.99831 |
| Adj. R-Square | 0.99803 |

### Example 5. Molecular Radio Afterglow Dynamic Probe (MRAP)

### CatB Selectivity

MRAP (100 M) was incubated with different enzymes including ALP (5 U ml⁻¹), β-gal (5 U ml⁻¹), furin (5 U ml⁻¹), NTR (5 μg ml⁻¹), GGT (5 μg ml⁻¹), CatB (5 μg ml⁻¹), APN (5 U ml⁻¹) and uPA (5 U ml⁻¹) for 6 h in their optimized buffer conditions according to the corresponding protocol from R&D Systems.

### Cell RAI

To study the cellular RAI, MRAP (100 μM) was added to different cell counts (1, 2, and 4×10⁴) of U87 or 3T3 medium for 12 h incubation, followed by X-ray irradiation (5 mGy, 250 μGy s⁻¹ for 20 s). The inhibitor group was pretreated with a CatB inhibitor (CA-074, 1 mM) for 10 h. The radio afterglow signals from each well were recorded with an IVIS imaging system with an open filter (120 s acquisition time).

### Results and Discussion

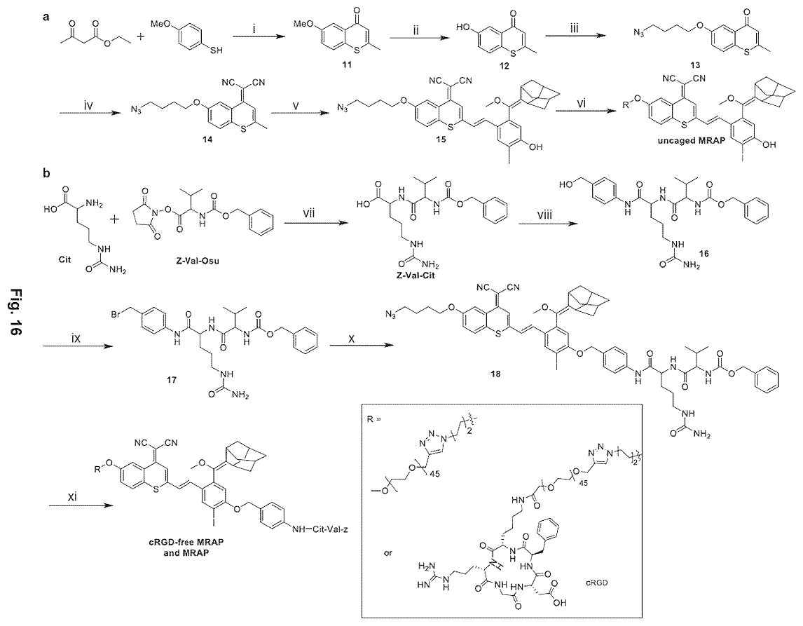

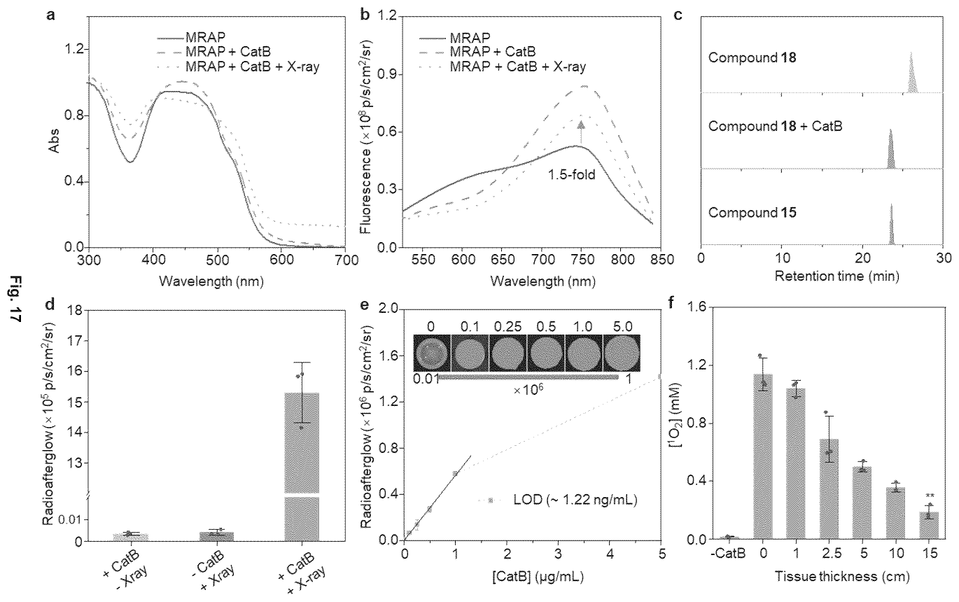

To enable precision cancer diagnosis and therapy, IDPA_{Su} was modified into a MRAP (as described in Example 1) that only activated its radio afterglow and radiodynamic functions in the presence of a tumour biomarker, namely, cathepsin B (CatB) (FIGS. 15a and 16). CatB was chosen because it is upregulated in multiple cancer cells, playing a role in tumour growth and progression. MRAP comprises three key units: a tumour-targeting moiety (cRGD), a radio afterglow dynamic theranostic moiety and a CatB-cleavable peptide moiety (Cit-Val). In the absence of CatB, MRAP had negligible radio afterglow and radiodynamic ¹O₂ generation (FIGS. 15b,d), because the electron-donating ability of the oxygen atom of the phenol unit in the phenoxy-adamantylidene unit was diminished, inhibiting its intramolecular charge transfer. In the presence of CatB, the peptide moiety was cleaved to liberate uncaged MRAP as confirmed by HPLC (FIG. 17c), leading to the activated radio afterglow at ~770 nm with 312-fold signal enhancement (FIGS. 15b and 17d). MRAP showed a superior selectivity towards CatB over other enzymes such as alkaline phosphatase (ALP), aminopeptidase N (APN), furin, gamma-glutamyl transferase (GGT), nitroreductase (NTR), β-galactosidase (β-gal) and urokinase plasminogen activator (uPA) (FIG. 15b). Moreover, the radio afterglow intensity of MRAP had good linearity with the concentration of CatB, with a low LOD of ~1.22 ng ml⁻¹ (FIG. 17e). The biomarker-activatable radiodynamic effect of MRAP was validated by ESR, showing the ESR signal of ¹O₂ increase by 7.8-fold on the incubation of MRAP with CatB followed by X-ray irradiation (FIGS. 15d and 17f).

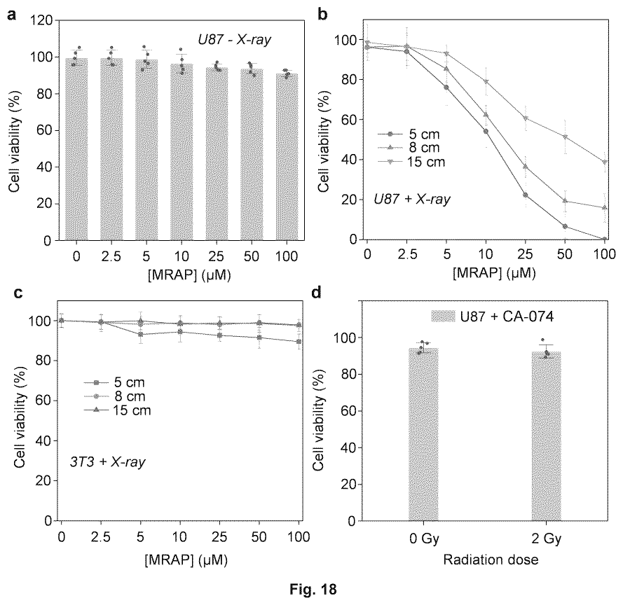

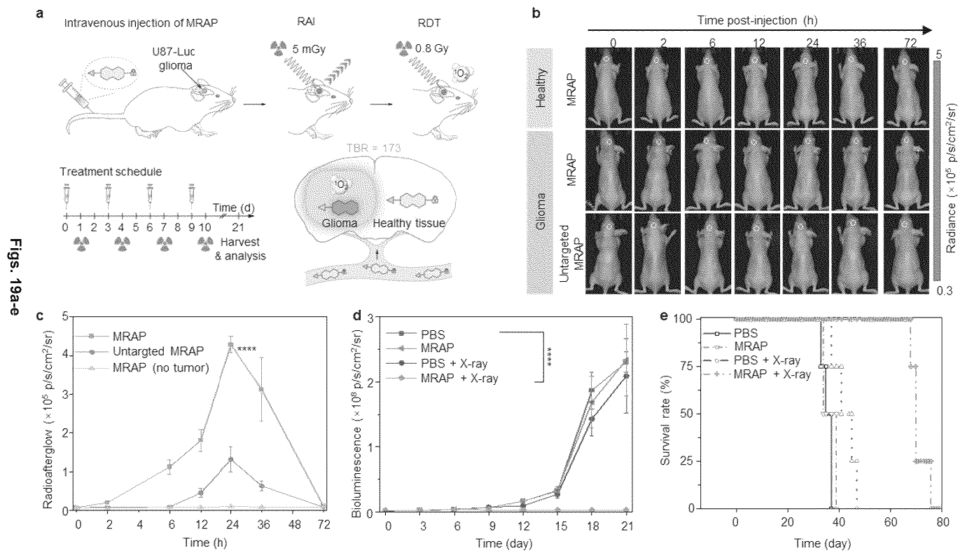

After confirming its good cytocompatibility (FIG. 18a), the biomarker specificity of MRAP was tested in U87 glioblastoma cells (CatB positive) and 3T3 fibroblast cells (CatB negative) (FIG. 15e). After incubation with MRAP and radiation, strong radio afterglow was observed for U87 cells (FIG. 15f), which was in sharp contrast to the signal for 3T3 cells and U87 cells pretreated with CA-074 (a CatB inhibitor), showing the signal as low as the background. At the cell counts of 4×10⁴ per well, the radio afterglow intensity of U87 cells was 78.5-fold higher than that of the 3T3 cells. Following X-ray irradiation, the cell viability of CatB-overexpressed cells decreased with increasing MRAP concentration, whereas no obvious toxicity was observed for cells with low CatB expression even at a high concentration of MRAP (100 M) (FIG. 15g). For example, the cell viability of U87 cells decreased with the increased MRAP concentration, reaching 38.2% at 10 M; in contrast, the cell viability of MRAP-treated 3T3 cells remained close to 100% regardless of the MRAP concentration. Thus, MRAP was only activated in U87 cells, verifying that its radio afterglow and radiodynamic toxicity was specific to CatB. This molecular radio afterglow dynamic selectivity of MRAP was also observed for solutions (FIG. 17f) and cells (FIGS. 18b-d) covered with a 15-cm-thick tissue, showing its potential for precision deep-tissue cancer theranostics.

### Example 6. In Vivo Cancer Radio Afterglow Dynamic Theranostics

### Pharmacokinetics Studies

Healthy BALB/c nude mice (six weeks old, female) were intraperitoneally injected with ketamine (50 mg kg⁻¹) for anaesthesia. Each group has three mice. At different timepoints (0.6, 1.2, 6.0, 30.0, 60.0, 120.0, 240.0, 480.0, 720.0 and 1,440.0 min) after an intravenous injection with MRAP or cRGD-free MRAP (10 μmol kg⁻¹), blood was collected from the eye socket vein and stored in heparin tubes in an ice bath. The plasma was obtained by blood sample centrifugation at 1,200×g for 20 min. The blood concentrations of MRAP and cRGD-free MRAP were determined by HPLC analysis, and their plasma half-life (t_{1/2}) was calculated.

Blood analyses were conducted with an automatic biochemical analyser (Chemray 240).

### Tumour Implantation

To establish a subcutaneous glioblastoma mouse model, BALB/c nude mice (six weeks old, female) were anaesthetized with isoflurane (2.0%) and subcutaneously injected with human U87 or U87-Luc (2×10⁶ per mouse) suspended in PBS (0.01 M, pH 7.4) on the left flank or bilateral flank of each mouse. The tumour size was calculated according to the formula V=½ab², where a and b are the longest and shortest diameter of the tumour, respectively. After tumour sizes reached ~70 mm³, the mice were used for imaging and therapy. To establish the orthotopic glioblastoma mouse model, BALB/c nude mice (six weeks old, female) were anaesthetized with isoflurane (2.0%) and fixed in a stereotactic instrument (RWD Life Science, E03143) for a skin incision on the brain region of interest: +0.5 mm anterior-posterior, +0.2 mm medial-lateral, −2.8 mm dorsal-ventral relative to the bregma. Then, human U87-Luc cells (2×10⁵ per mouse) suspended in PBS (0.01 M, pH 7.4) were implanted through a burr hole with a size of 1 mm² by a microsyringe. The injection process lasted for 15 min. The burr hole was sealed by bone wax, and the skin incision was closed with surgical clips. To monitor the tumour growth, mice were intraperitoneally injected with d-luciferin sodium salt (500 μmol kg¹). At 15 min post-injection, bioluminescence signals were recorded using an IVIS imaging system with an open filter and automatic acquisition time. Mice were used for imaging and therapy after the bioluminescent signal of orthotopic tumours reached up to ~1-2×10⁶ photons s⁻¹ cm⁻² sr⁻¹.

### Biodistribution Studies

Orthotopic U87-tumour-bearing mice were intravenously injected with MRAP or cRGD-free MRAP (10 μmol kg⁻¹). At 24 h post-injection, the mice were sacrificed and major organs and tumours were collected for homogenization in PBS (0.01 M, pH 7.4). The homogenates were

centrifuged at 1,585×g for 20 min to remove the insoluble components. The supernatants containing MRAP or cRGD-free MRAP were analysed by HPLC. MRAP or cRGD-free MRAP concentration in tissues was calculated by the calibration curves obtained from its solutions of known concentrations, and converted into percent of injected dose per gram of tissue (% ID g⁻¹).

### RDI Studies

For in vivo RAI by IDPA_{Su}, subcutaneous U87-tumour-bearing mice were intratumorally injected with IDPA_{Su}(100 μM, 20 kl) solubilized with P-cyclodextrin (100 μM). Each group has three mice. At different post-injection timepoints (0, 0.5, 1.0, 2.0, 4.0, 6.0, 8.0, 12.0, 16.0 and 24.0 h), the tumours were irradiated with X-rays (5 mGy, 250 μGy s⁻¹ for 20 s), followed by radio afterglow detection using the IVIS imaging system under the bioluminescence mode with an open filter and acquisition time of 120 s. Tumour-free areas were protected by a 3.4-mm-thick lead shield with a notch. For in vivo RAI by MRAP, MRAP or cRGD-free MRAP (10 μmol kg⁻¹) were intravenously injected into orthotopic U87-tumour-bearing mice. At different post-injection times (0, 2, 6, 12, 24, 36 and 72 h), mouse brains were exposed to X-ray irradiation and the other areas were protected by a 3.4-mm-thick lead shield with a notch. Radio afterglow signals from brain tumours were recorded with the IVIS imaging system under the bioluminescence mode with an open filter and acquisition time of 120 s. For ultrasmall-tumour detection, U87-Luc cells (2×10⁶ per mouse) were subcutaneously implanted into the left flank of each mouse. The tumour sizes were calculated as per the above-mentioned equation, showing ~0 mm³ for 1 day post-implantation, 6 mm³ (tumour diameter, 2.3 mm) for 5 days post-implantation, 22 mm³ for 10 days post-implantation, 43 mm³ for 20 days post-implantation and 77 mm³ for 30 days post-implantation. At 15 min post-intraperitoneal injection of aqueous d-luciferin sodium salt (500 μmol kg⁻¹) solution into mice with different tumour sizes, the bioluminescence signals from the tumour sites were recorded with the IVIS imaging system under the bioluminescence mode with an open filter and automatic acquisition time. At 4 h post-intraperitoneal injection of d-luciferin sodium salt, no bioluminescence signal was observed, followed by an intravenous injection of MRAP (10 μmol kg⁻¹) in U87-Luc-tumour-bearing mice. At 20 h post-injection of MRAP, radio afterglow signals from tumour sites after X-ray irradiation (5 mGy, 250 μGy s⁻¹ for 20 s) were recorded by the IVIS imaging system under the bioluminescence mode with an open filter and acquisition time of 120 s.

### RDT Studies

For subcutaneous tumour therapy, to study the X-ray dynamic activity of IDPA_{Su}, U87-tumour-bearing mice were randomly divided into four groups (PBS, PBS+X-ray, IDPA_{Su} and IDPA_{Su}+X-ray), which includes five mice per group. On day 0, tumours were intratumorally injected with PBS or IDPA_{Su} (0.1 μmol kg⁻¹, solubilized with 0.1 μmol kg⁻¹ of β-CD) and irradiated with X-rays (0.1 Gy, 5 mGy s⁻¹ for 20 s) at 30 min post-injection. During the therapy, the mice were anaesthetized by intraperitoneally injected ketamine (50 mg kg⁻¹) in saline. Such therapy was performed every three days. Only the tumour site was irradiated by X-rays as the other areas were protected by a 3.4-mm-thick lead shield with a notch. The tumour volume and body weight of mice were monitored every two days. The tumours and main organs were collected for histological analysis after therapy (H&E and TUNEL staining). For orthotopic glioblastoma therapy, orthotopic U87-tumour-bearing mice were randomly divided into four groups (PBS, PBS+X-ray, MRAP and MRAP+X-ray; n=5 mice per group). On day 0, mice were intraperitoneally injected with d-luciferin sodium salt and PBS (0.01 M, 200 μl) for bioluminescence imaging. After 4 h when no bioluminescence signal of luciferin on the brain was observed, the mice were intravenously injected with or without MRAP (10 μmol kg⁻¹) and not irradiated or irradiated with X-rays (0.8 Gy, 40 mGy s⁻¹ for 20 s) at 24 h post-injection. The whole brain was irradiated by X-rays with a binning area of 6.03 cm² as the other areas were protected by a 3.4-mm-thick lead shield with a notch during X-ray therapy. To avoid lethal brain oedema, dexamethasone (5 mg kg⁻¹) was intramuscularly injected at the right hind limb of each mouse at 18 h before and 6 h after X-ray irradiation. During the therapy, mice were anaesthetized by intraperitoneally injected ketamine (50 mg kg⁻¹) in saline. The brain tumour size was monitored using bioluminescence signals of luciferin from the tumour site every three days. Mice were euthanized when they either lost 20% of their body weight or presented any obvious signs and symptoms of sickness during the therapy. Tumours and main organs were collected for histological analysis after therapy (H&E and caspase-1 staining). For precision and biocompatibility studies, MRAP (0.5 μmol kg⁻¹) was intracranially injected into the brain of orthotopic U87-tumour-bearing mice through a burr hole with a size of 1 mm² by a microsyringe. For comparison, uncaged MRAP (0.5 μmol kg⁻¹) was intracranially injected into the brain of healthy mice. At 30 min post-injection of MRAP or uncaged MRAP, both in vivo and ex vivo radio afterglow signals from the brain after X-ray irradiation (2 Gy, 100 mGy s⁻¹ for 20 s) were recorded by the IVIS imaging system under the bioluminescence mode with an open filter and acquisition time of 120 s. Only brains were exposed to X-ray irradiation and the other body areas were protected by a 3.4-mm-thick lead shield with a notch. Furthermore, the brains were fixed and sectioned for histological analysis (H&E and anti-cleaved caspase-3 staining).

### Results and Discussion

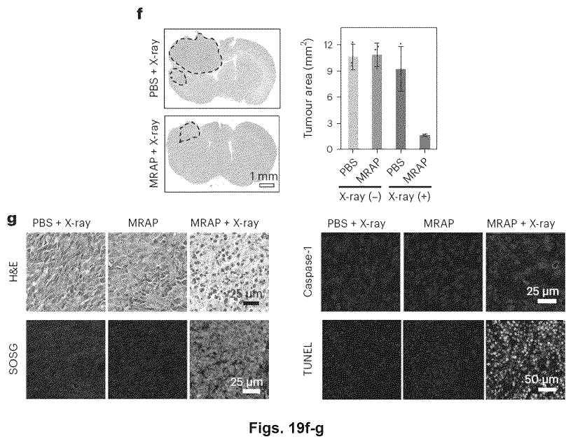

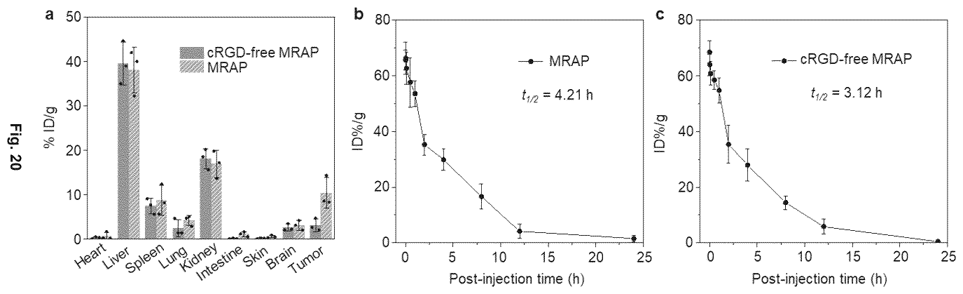

The ability of MRAP for cancer radio afterglow dynamic theranostics was validated in intracranial glioblastoma mouse model using luciferase-expressed U87 cancer cell line (U87-Luc). After 14 days of U87-Luc tumour implantation, longitudinal RAI was conducted via the intravenous injection of MRAP or cRGD-free MRAP into the U87-tumour-bearing mice (FIG. 19a). The radio afterglow signals for both MRAP and cRGD-free MRAP gradually increased and reached the maximum at 24 h post-injection (FIGS. 19b-c), showing the tumour-to-background ratios (TBRs) of ~173 and 38, respectively. In contrast, the signal remained as low as the background for tumour-free healthy mice. The higher TBR of MRAP relative to cRGD-free MRAP was attributed to the targeting capability of cRGD towards αvβ3 integrin overexpressed in the tumour, which was also verified by the higher percent injected dose (ID %) of MRAP (10.4%) relative to cRGD-free MRAP (3.1%) at 24 h post-injection (FIG. 20a). Then, the pharmacokinetics of both probes and fragments were studied (FIGS. 20b-c). The plasma elimination half-life (t_{1/2}) of MRAP was ~4.2 h, slightly longer than that of cRGD-free MRAP (t_{1/2}=~3.1 h).

After confirming the highest signal 24 h post-intravenous injection of MRAP, MRAP-mediated RDI was scheduled on day 0, 3, 6 and 9 with a fraction dosage of 0.8 Gy (FIG. 19a).

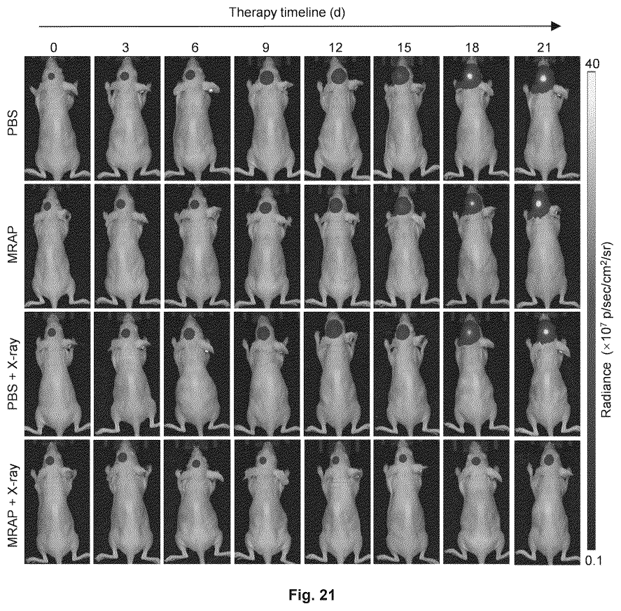

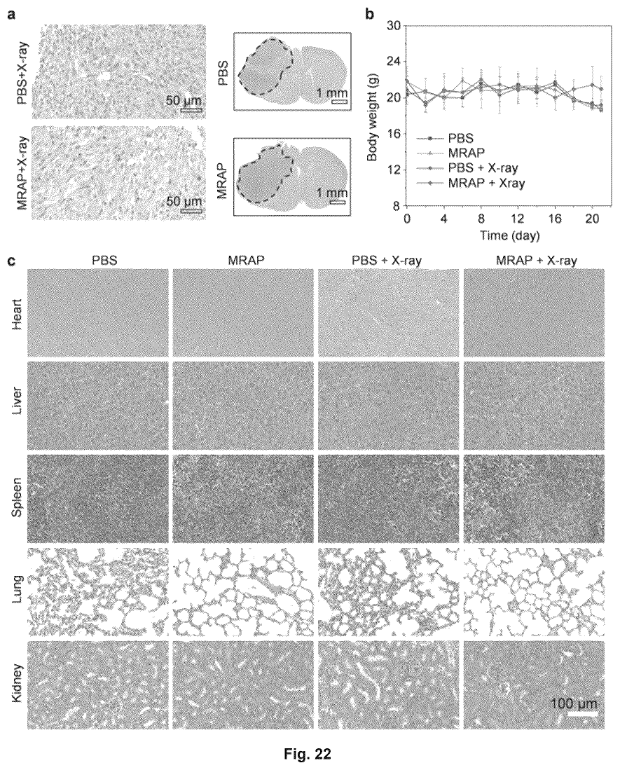

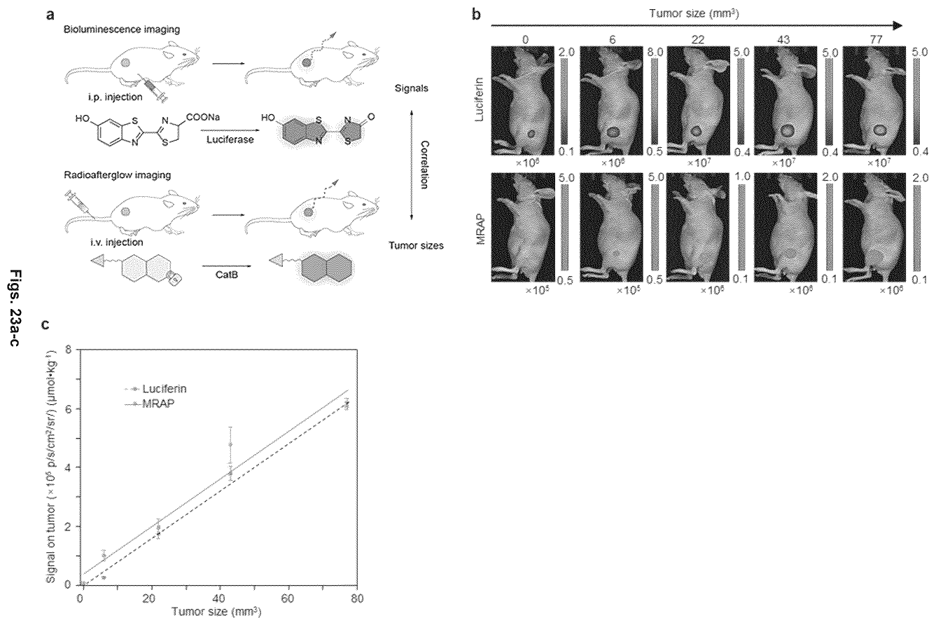

The tumour growth was continuously monitored by a bioluminescence signal from U87-Luc cells in mice intraperitoneally injected with d-luciferin (FIG. 21). After RDT for four times, the tumour growth was completely inhibited, whereas other groups showed no obvious regression or inhibition of the tumour growth (FIG. 19d). Therefore, the survival time for MRAP-mediated RDT groups were increased from 37 days (phosphate-buffered saline (PBS)) to 76 days, showing a 100% survival rate until 67 days (FIG. 19e). As verified by H&E staining on day 21, the tumour tissue was only ~1.61 mm² after MRAP-mediated RDT, which was ~5.74 times smaller than the PBS+X-ray group (~9.24 mm²) (FIG. 19f). Moreover, the density of cancer cells in the tumour tissue for MRAP-mediated RDT was much lower than the other groups (FIGS. 19g and 22a), showing obvious cell death. SOSG immunofluorescence staining confirmed the effective in situ generation of ¹O₂ by MRAP in the tumour under X-ray irradiation (FIG. 19g). Similarly, caspase-1 staining and TUNEL assay revealed strong signals from the MRAP-mediated RDT group, suggesting the pyroptosis and apoptosis of cancer cells. Note that no obvious loss in body weight and sign of tissue damage was detected in major organs of mice after MRAP-mediated RDT (FIGS. 22b-c).

### Example 7. Sensitivity and Precision Studies

The sensitivity of MRAP for cancer detection was tested in a subcutaneous U87-Luc-tumour-bearing mouse model. MRAP was intravenously injected into mice bearing U87-Luc tumours with different sizes, whereas the bioluminescence substrate (d-luciferin) was intraperitoneally

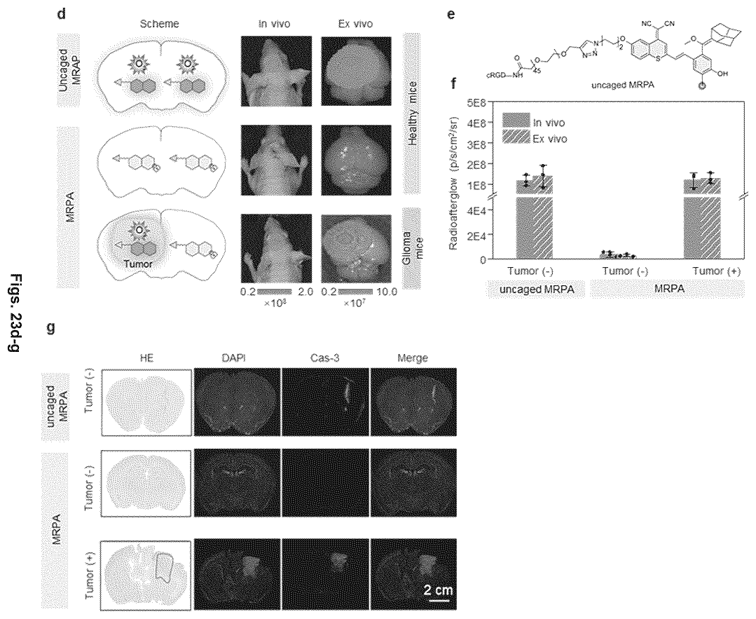

injected for comparison by following the protocols in Example 6 (FIGS. 23a-b). In accordance with bioluminescence imaging, RAI had a linear signal correlation with the tumour size but slightly higher intensities than bioluminescence (FIG. 23c). The LOD for tumour detection was determined to be 1.08 mm³ (diameter, 0.64 mm), which was within the detection range for micrometastatic tumours (<2.00 mm).

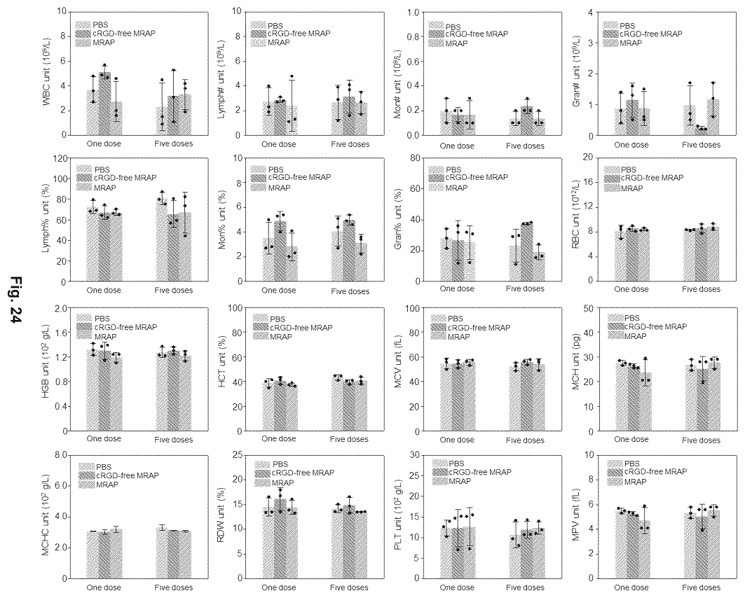

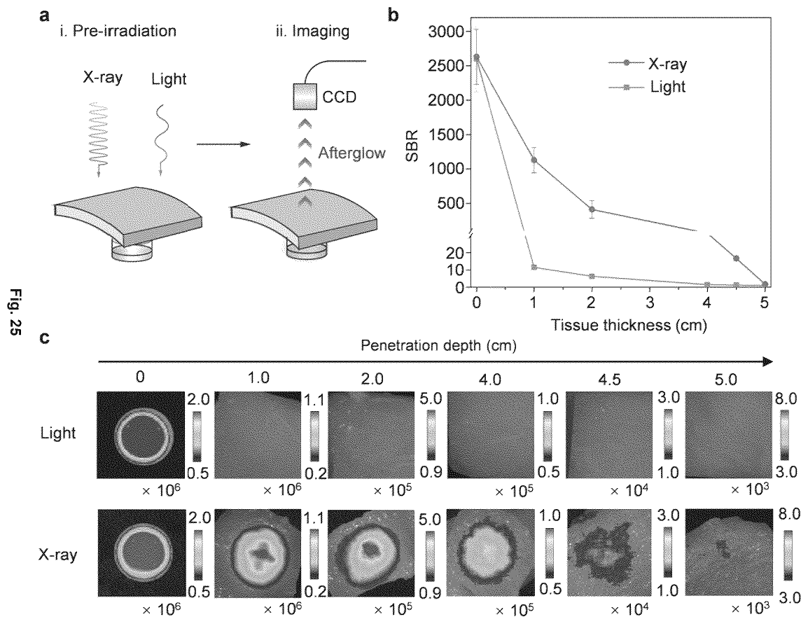

The precision of MRAP-mediated RDT was evaluated and compared with an ‘always-on’ control probe (FIG. 23e, uncaged MIRAP). After confirming the ideal blood compatibility for both MRAP and cRGD-free MRAP at a high total dosage (100 μmol kg⁻¹) by a routine blood analysis by following the protocol in Example 6 (FIG. 24), the probe was intracranially injected into mice with or without intracranial glioblastoma. Due to the CatB selectivity, MRAP was only activated to emit a strong signal in the tumour, whereas its signal remained low for a tumour-free healthy mouse (FIG. 23d); in contrast, the signal was always-on for the control probe in the healthy mouse. This was consistent with ex vivo brain imaging (FIGS. 23d,f). After X-ray irradiation, the mice were immediately sacrificed for tissue analysis. Due to the tumour specificity, the radiodynamic function of MRAP remained inactive in the brain of healthy mouse, which was not possible for the control probe. Thus, MRAP only resulted in tissue damage and apoptotic caspase-3 signal in the tumour area of the glioblastoma mouse (FIG. 23g); in contrast, the ‘always-on’ radiodynamic function of the control probe triggered the non-specific cellular apoptosis in the brain of healthy mice. These data confirmed the high sensitivity and precision of MRAP in cancer therapy.

The utilization of X-rays to induce the photophysical processes of optical agents in deep tissues led to the emergence of RAI and RDT. These hybrid modalities not only tackle the tissue penetration issue of optical imaging and cancer phototherapy but also minimize the radiation damage to normal tissues. However, only inorganic nanophosphors are available for both applications, and their radio afterglow dynamic function is always on, limiting their specificity and efficacy in cancer detection and treatment.

To enable radio afterglow for organic molecules, we proposed a radiodynamic approach based on organic luminophores (DPAs and IDPAs) that were able to harvest X-ray energy for an in situ cycloaddition reaction to form self-luminescent chemical defects (FIG. 4b). This is mechanistically different from inorganic nanophosphors wherein the host materials (for example, fluoride and aluminosilicate) absorb X-rays, lattice defects delay excitons, and rare-earth/transition metal ion dopants (for example, Nd³⁺ and Eu³⁺) emit light (Richard, C. & Viana, B., Light.: Sci. Appl. 2022, 11, 123). Because of the presence of heavy atoms (iodine), IDPAs had higher radiodynamic ¹O₂ generation and stronger radio afterglow emission than the iodine-free counterpart (DPAs). The radio afterglow dynamic processes of IDPAs were inducible even at a tissue depth of 15 cm, which was impossible when using light. Moreover, IDPAs had the afterglow half-life (18.4 min) comparable with inorganic nanophosphors, but 25.4 times higher in vivo intensity based on the same mass (1.27×10⁶ versus 5.00×10⁴ photons s⁻¹ cm⁻² sr⁻¹ μg⁻¹) (Table 3); such a higher brightness resulted in the induction and detection of radio afterglow signal in the tissue depth 2.5 times deeper than inorganic nanophosphors (5.0 versus 2.0 cm) (FIG. 24).

TABLE 3
Summary of X-ray-induced NIR afterglow for in vivo imaging.

| | | | | | | Penetration depth | |
|---|---|---|---|---|---|---|---|
| | | | | | | Detection | Induction |
| | | | | | | with tissue | and |
| | | | | | X-ray | (induction | detection |
| | | λₑₘ | Half- | Life- | dosage | without | through |
| | Material | (nm) | life | time | (Gy) | tissue) | tissue |
| Inorganic | NC-LGO:Cr | ~720 | minutes | 5 | 0.02 | N.A. | 1.5 |
| materials | @mSiO₂ | nm | | h | | | cm |
| | ZnGa₂O₄:Cr | 700 | minutes | 6 | 0.00103 | N.A. | 2 |
| | | nm | | h | | | cm |
| | ZnGa₂O₄:Cr/W | 696 | minutes | hours | 0.18 | N.A. | 2.0 |
| | | nm | | | | | cm |
| | Concave | 690 | ~100 | 360 | 0.5 | N.A. | N.A. |
| | ZnGa₂O₄:Cr³⁺ | nm | s | min | | | |
| | | (c.a.) | | | | | |
| | mZGGOs and | ~710 | hours | 8 h | 1 | N.A. | 0.9 |
| | Si- | nm | | | | | cm |
| | Pc@PEmZGGOs | | | | | | |
| | Mn-ZGGOs | 696 | <1 | 120 | 0.1 | N.A. | 1.0 |
| | | nm | h | min | | | cm |
| | NaYF₄:Er | 1525 | minutes | >72 | 200 | N.A. | 0.2-0.4 |
| | @NaYF₄ | nm | | h | | | cm |
| | MgGeO₃:Yb²⁺, | 670 | minutes | hours | 0.025 | N.A. | 1.5 |
| | Yb³⁺@SiO₂ | nm, | | | | | cm |
| | | 1019 | | | | | |
| | | nm | | | | | |
| Organic | IDPA_{Su} | ~773 | 18.4 | 25 | 0.005 | 15 | 5 |
| materials | | nm | mins | h | | cm | cm |
| | cRGD-free | ~770 | N.A. | N.A. | 0.005 | N.A. | N.A. |
| | MRAP | nm | | | | | |
| | MRAP | ~770 | N.A. | N.A. | 0.005 | N.A. | N.A. |
| | | nm | | | | | |

| | local | Intravenous | | |
|---|---|---|---|---|
| | injection | injection | | |
| | Intensity | Intensity | | |
| | per dose | per dose | | |
| | per mouse | per mouse | SBR | Ref. |
| Inorganic | 5 × 10⁴ | 2.3 × 10³ | N.A. | Mater. Horiz. |
| materials | (p/s/cm²/sr)/ | (p/s/cm²/sr)/μg | | 2017, 4, |
| | (μg) | (H1299 | | 1092-1101 |
| | (healthy | orthotopic | | |
| | nude mice) | tumor-bearing | | |
| | | nude mice) | | |
| | ~1100 | N.A. | N.A. | ACS Appl. Mater. |
| | (p/s/cm²/sr)/ | | | Interfaces |
| | (μg) | | | 2017, 9, |
| | (healthy | | | 22132-22142 |
| | kunming | | | |
| | mice) | | | |
| | N.A. | N.A. | N.A. | Adv. Funct. Mater. |
| | | | | 2018, 28, |
| | | | | 1707496 |
| | N.A. | 8.25 | N.A. | Adv. Mater. |
| | | (p/s/cm²/sr)/μg | | 2019, 31, |
| | | (HepG2 | | 1905087 |
| | | orthotopic | | |
| | | tumor-bearing | | |
| | | nude mice) | | |
| | 100 | 8 × 10⁴ | N.A. | Adv. Funct. Mater. |
| | (p/s/cm²/sr)/ | (p/s/cm²/sr)/μg | | 2020, 30, |
| | (μg) | (HepG2 | | 2001166 |
| | (healthy | orthotopic | | |
| | nude mice) | tumor-bearing | | |
| | | nude mice) | | |
| | N.A. | ~3.9×10⁴ | N.A. | Theranostics |
| | | (p/s/cm²/sr)/μg | | 2021, 11, |
| | | (U87MG | | 7439-7449 |
| | | subcutaneous | | |
| | | tumor-bearing | | |
| | | Balb/c nude | | |
| | | mice) | | |
| | N.A. | N.A. (4T1 | 40.9 | Nat. Nanotechnol. |
| | | subcutaneous | (Balb/c | 2021, 16, |
| | | tumor-bearing | mice) | 1011-1018 |
| | | Balb/c nude | | |
| | | mice) | | |
| | N.A. | 8.5×10³ | | ACS Appl. |
| | | (p/s/cm2/sr)/μg | 5.8 | Mater. Interfaces. |
| | | (HepG2 | | 2021, 13, |
| | | subcutaneous | | 16166-16172 |
| | | tumor-bearing | | |
| | | Balb/c nude | | |
| | | mice) | | |
| Organic | 1.27 × 10⁶ | N.A. | N.A. | The present |
| materials | (p/s/cm2/sr)/ | | | disclosure |
| | (μg) | | | |
| | (U87MG | | | |
| | subcutaneous | | | |
| | tumor-bearing | | | |
| | Balb/c nude | | | |
| | mice) | | | |
| | N.A. | 6.62 × 10⁵ | ~38 | |
| | | (p/s/cm2/sr)/μmol | | |
| | | (U87MG | | |
| | | orthotopic | | |
| | | tumor-bearing | | |
| | | Balb/c nude | | |
| | | mice) | | |
| | ~6.68 × 10⁶ | 3.10 × 10⁶ | ~234 | |
| | (p/s/cm2/sr)/ | (p/s/cm²/sr)/μmol | | |
| | (μg) | (U87MG | | |
| | (2 Gy dose) | subcutaneous | | |
| | (U87MG | tumor-bearing | | |
| | orthotopic | Balb/c nude | | |
| | tumor-bearing | mice) | | |
| | Balb/c nude | 2.14 × 10⁶ | 173 | |
| | mice) | (p/s/cm2/sr)/μmol | | |
| | | (U87MG | | |
| | | orthotopic | | |
| | | tumor-bearing | | |
| | | Balb/c nude | | |
| | | mice) | | |

Note:
SBR: signal-to-background ratio; N.A: not available; Ref.: reference.

To achieve high cancer theranostic specificity, a candidate radio-luminophore (IDPA_{Su}) was developed into a smart probe (MRAP), which only turned on its radio afterglow dynamic function in the presence of a cancer biomarker (CatB). Such a biomarker-activated radio afterglow of MRAP led to the ultrahigh sensitivity, showing a TBR value that was ~5.7 times higher even than a second near-infrared radiodynamic nanophosphor (NaYF₄:Er@NaYF₄) for the imaging of a subcutaneous tumour (234 versus 41) (Pei, P. et al., Nat. Nanotechnol. 2021, 16, 1011-1018). Therefore, MRAP-based RAI detected the ultrasmall tumour (6 mm³, 2.3 mm in diameter), more sensitive than clinical imaging methods (>7.0 mm) and circulating-tumour DNA sensors (>10.0 mm) (Pashayan, N. & Pharoah, P. D. P., Science 2020, 368, 589-590).

As exemplified by the comparison between MRAP and the always-on probe (FIG. 23d), the biomarker activation of MRAP ensured that the radiodynamic toxicity specifically occurred in the tumour tissue and spared the adjacent normal tissues despite the co-existence of MRAP. This is intrinsically superior over existing agents whose radio therapeutic effect is mainly dictated by their distribution and local concentration in tissues. In conjugation with the higher radiodynamic efficiency (>5.7 times higher than the commercially available radio sensitizers; FIG. 4i), IDPASu and MRAP-mediated RDT completely inhibited the tumour growth in subcutaneous and orthotopic glioblastoma mouse models at the respective total dosages of 0.3 Gy (0.038 mg) and 3.2 Gy (2.960 mg), which were much lower than other reported RDT agents, namely, >0.5 Gy (0.125 mg) and >20.0 Gy (>8.000 mg), respectively (Table 4).

TABLE 4
Summary of X-ray-induced NIR afterglow for in vivo imaging.

| | Scintillator/ | | Injection | | | | | |
|---|---|---|---|---|---|---|---|---|
| | Transducer/ | | dosage | | X-ray dosage | Radiation | Tumor | |
| Radiation | radiosensitizer | Photosensitizer | (per mouse) | | (Gy) | mechanism | model | Ref. |
| Conventional | — | — | — | | total 60 | water | Orthotopic | J. Neurosci. |
| radiotherapy | | | | | (2 Gy × 30 | radiolysis | brain tumor | Methods |
| | | | | | fractions) | | (Pangenic | 2003, 125, |
| | | | | | | | Swiss nu/nu | 147-157 |
| | | | | | | | mice) | |
| | — | — | — | | total 10-20 | water | Orthotopic | J. Clin. Invest. |
| | | | | | (2-4 Gy × 5 | radiolysis | glioblastoma | 2010, 120, |
| | | | | | fractions) | | (nude mice) | 694-705 |
| X-PDT therapy | AuNPs | — | 80 mg ([Au]) | | total 30-35 | ROS | Tu-2449 | Nanomedicine |
| (transducer/ | | | | | (30-35 Gy × | | orthotopic | 2013, 8, |
| nanoscintil- | | | | | 1 fraction) | | brain tumor | 1601-1609 |
| lator + | | | | | | | (I.V.) (black | |
| photosensitizer) | | | | | | | B6C3f1 mice) | |
| | PEGylated- | — | 8 mg ([Au]) | | total 20 (20 | ROS | U251 | PLoS One |
| | gold | | | | Gy × 1 | | orthotopic | 2013, 8 |
| | nanoparticles | | | | fraction) | | Glioblastoma | e62425 |
| | (GNPs) | | | | | | tumor | |
| | | | | | | | (I.V.) | |
| | AgNPs | — | 0.01 | mg | total 10 (10 | ROS | Rat C6 | Nanoscale |
| | | | | | Gy × 1 | | glioma | 2013, 5, |
| | | | | | fraction) | | Orthotopic | 11829-11836 |
| | | | | | | | Glioblastoma | |
| | | | | | | | tumor | |
| | | | | | | | (Adult male | |
| | | | | | | | Wistar rats) | |
| | | | | | | | (I.P.) | |
| | GBNs | — | 1 | μmol | total 10 (10 | ROS | SQ20B | Nanomed.: |
| | (GdNs) | | | | Gy × 1 | | subcutaneous | Nanotechnol. |
| | | | | | fraction) | | tumor | Biol. Med. |
| | | | | | | | (athymic | 2015, 11, |
| | | | | | | | nude mice) | 247-257 |
| | | | | | | | (I.T.) | |
| | PAA- | — | 0.013 | mg | total 5 (5 | •OH | MIAPaCa-2 | Radiat. Oncol. |
| | TiOxNPs | | | | Gy × 1 | | subcutaneous | 2016, 11, 91 |
| | | | | | fraction) | | tumor | |
| | | | | | | | (balb/c nude | |
| | | | | | | | mice) (I.T.) | |
| | BSA-coated | — | 0.04 | mg | total 6 (6 | •OH and | BEL-7402 | Adv. Mater. |
| | BiOI@Bi₂S₃ | | | | Gy × 1 | O2^{•-} | subcutaneous | 2017, 29, |
| | | | | | fraction) | | tumor | 1704136 |
| | | | | | | | (balb/c nude | |
| | | | | | | | mice) (I.T.) | |
| | | | | | | | SMMC-7721 | Int. J. |
| | HA-Bi₂O₃ | — | ~0.2 | mg | total 6 (6 | ROS | subcutaneous | Nanomed. |
| | NPs | | | | Gy × 1 | | tumor (ICR | 2017, 12, |
| | | | | | fraction) | | mice) (I.V.) | 5973 |
| | pHLIP-Cu- | — | ~0.016 | mg | total 5 (5 | ¹O₂ | CRL-2116 | Proc. Natl. Acad. |
| | Cy | | | | Gy × 1 | | subcutaneous | Sci. U.S.A. |
| | NPs | | | | fraction) | | tumor | 2019, 116, |
| | | | | | | | (BALB/c) | 16823-16828 |
| | | | | | | | (I.T.) | |
| | T-BiO₂₋ₓ | — | 0.1 | mg | total 8 (8 | •OH and | 4T1 | Inorg. Chem. |
| | | | | | Gy × 1 | O2^{•-} | subcutaneous | 2020, 59, |
| | | | | | fraction) | | tumor | 3482-3493 |
| | | | | | | | (BALB/c | |
| | | | | | | | mice) (I.T.) | |
| | ES-GON- | — | 0.1 mg ([Gd]) | | total 6 (6 | ROS | U87MG | Biomaterials |
| | rBSA-LF- | | | | Gy × 1 | | subcutaneous | 2020, 235, |
| | RGD2 | | | | fraction) | | tumor (I.V.) | 119783 |
| | (Gd₂O₃ NPs) | | | | | | | |
| | TiO₂ | — | total 0.2 mg | | total 8 (4 | •OH | U87MG | J. |
| | (TiO₂:Bi) | | | | Gy × 2 | | subcutaneous | Nanobiotechnology |
| | | | | | fractions) | | tumor (balb/c | 2022, 20, 293 |
| | | | | | | | nude mice) | |
| | | | | | | | (I.V.) | |
| | Cu-Cy NPs | — | 0.05 mg | | total 2 (2 | ¹O₂ | 4T1 | Bioact. Mater. |
| | | | (mice) | | Gy × 1 | | subcutaneous | 2022, 7, |
| | | | | | fraction) | | tumor | 504-514 |
| | | | | | | | (BALB/c | |
| | | | | | | | nude mice) | |
| | | | | | | | (I.T.) | |
| | LiYF₄:Ce | ZnO | 0.1 | mg | total 8 (8 | •OH and | Hela | Angew. Chem. |
| | | | | | Gy × 1 | O2^{•-} | subcutaneous | Int. Ed. Engl. |
| | | | | | fraction) | | tumor | 2015, 54, |
| | | | | | | | (BALB/c | 1770-1774 |
| | | | | | | | nude mice) | |
| | | | | | | | (I.T.) | |
| | SrAl₂O₄:Eu | MC540 | 0.125 | mg | total 0.5 (0.5 | ¹O₂ | U87MG | Nano Lett. |
| | | | | | Gy × 1 | | subcutaneous | 2015, 15, |
| | | | | | fraction) | | tumor (balb/c | 2249-2256 |
| | | | | | | | nude mice) | |
| | | | | | | | (I.T.) | |
| | NaYF4:Yb:Gd:Er:Tm | SiPc(OH)₂; | 0.4 | mg | total 6 (6 | •OH and | HT-1080 | Biomaterials |
| | | PpIX | | | Gy × 1 | O2^{•-} | subcutaneous | 2015, 69, |
| | | | | | fraction) | | tumor (balb/c | 89-98 |
| | | | | | | | nude mice) | |
| | | | | | | | (I. V.) | |
| | SrAl₂O₄:Eu²⁺ | MC540 | 0.085 mg | | total 5 (5 | ¹O₂ | H1299-Luc | Theranostics |
| | | | (subcutaneous | | Gy × 1 | | subcutaneous | 2016, 6, |
| | | | tumor); | | fraction) | | tumor | 2295-2305 |
| | | | 0.125 mg | | | | (I.T.) | |
| | | | (lung | | | | H1299-Luc | |
| | | | orthotopic | | | | orthotopic | |
| | | | tumor) | | | | tumor (I.T.) | |
| | | | | | | | (Harlan nude | |
| | | | | | | | mice) | |
| | Hf₁₂ and Hf₆ | Ir(bpy)[dF(CF₃)ppy]²⁺ | 0.2 | μmol | total 2.5 (0.5 | ¹O₂, •OH | MC38 | J. Am. Chem. Soc. |
| | SBUs | | | | Gy × 5 | and O2^{•-} | subcutaneous | 2018, 140, |
| | | | | | fractions) | | tumor (I.T.) | 16971-16975 |
| | | | | | | | (C57BL/6 | |
| | | | | | | | mice) | |
| | ZnGa₂O₄:Cr/W | ZnPcS4 | total 0.6 mg | | total 0.36 (0.18 | ¹O₂ | S180 | Adv. Funct. Mater. |
| | | | | | Gy × 2 | | subcutaneous | 2018, 28, |
| | | | | | fractions) | | tumor (balb/c | 1707496 |
| | | | | | | | nude mice) | |
| | | | | | | | (I.V.) | |
| | Zn₂SiO₄:Mn | Rose bengal | total ~0.8 mg | | total 2 (1 | ¹O₂ | U87MG | Adv. Mater. |
| | | | | | Gy × 2 | | subcutaneous | 2019, 31, |
| | | | | | fractions) | | tumor (balb/c | 1808024 |
| | | | | | | | nude mice) | |
| | | | | | | | (I.V.) | |
| | Au NPs | verteporfin | 0.2 | mg | total 4-12 (4- | ¹O₂ | HCT 116 | ACS Cent. Sci. |
| | | | | | 12 Gy × 1 | | subcutaneous | 2020, 6, |
| | | | | | fraction) | | tumor (NOD | 715-726 |
| | | | | | | | scid gamma | |
| | | | | | | | mice) (I.T.) | |

| | | | | | | | |
|---|---|---|---|---|---|---|---|
| | Zn₃Ga₂GeO₈:Cr³⁺, | Si-Pc | total 0.8 mg | total 4 (2 | ¹O₂ | HepG2 | Adv. Funct. Mater. |
| | Yb³⁺, Er³⁺ | | | Gy × 2 | | subcutaneous | 2020, 30, |
| | (mZGGOs) | | | fractions) | | tumor (nude | 2001166 |
| | | | | | | mice) (I.V) | |
| | AIE-Au | Rose bengal | total 0.8 mg | total 1 (0.5 | •OH and ¹O₂ | U87MG | Angew. Chem. |
| | | | | Gy × 2 | | subcutaneous | Int. Ed. Engl. |
| | | | | fractions) | | tumor (balb/c | 2020, 132, |
| | | | | | | nude mice) | 10000-10007 |
| | | | | | | (I.V.) | |
| | Hf⁴⁺ | TPEDC-DAC | 0.12 mg | total 16 (8 | ¹O₂ and •OH | 4T1 tumor | Adv. Mater. |
| | | | (Hf) | Gy × 2 | | subcutaneous | 2021, 33, |
| | | | | fractions) | | tumor (Balb/c | 2007888 |
| | | | | | | mice) (I.V.) | |
| X-PDT therapy | Photofrin ® | — | ~0.17 μmol | total 5 (5 | N.A. | human cell | Onkologie |
| (Organic | | | (~0.2 mg) | Gy × 1 | | bladder | 2001, 24, |
| radiosensitizer) | | | | fraction) | | cancer | 482-485 |
| | | | | | | subcutaneous | |
| | | | | | | tumor (nude | |
| | | | | | | mice) (I.V.) | |
| | Acridine Orange | — | 0.829 μmol | total 5 (5 | ¹O₂ | Osteosarcomas | Oncology |
| | | | (0.22 mg) | Gy × 1 | | subcutaneous | 2002, 62, |
| | | | | fraction) | | tumor (C3H | 85-93 |
| | | | | | | mice) (I.P.) | |
| | 5-ALA (PpIX | — | ~53.7 μmol | total 30 (3 | ¹O₂ | B16 | Int. J. Radiat. Biol. |
| | precursor) | | (~0.9 mg) | Gy × 10 | | subcutaneous | 2016, 92, |
| | | | | fractions) | | tumor | 774-789 |
| | | | | | | (C57BL/6J | |
| | | | | | | mice) (I.T.) | |
| | ITC-NPs | — | ~0.06 μmol | total 0.4 (0.4 | ¹O₂ | 4T1 | Nat. Commun. |
| | | | (0.4 mg) | Gy × 1 | | subcutaneous | 2022, 13, |
| | | | | fraction) | | tumor (Balb/c | 5091 |
| | | | | | | mice) (I.V.) | |
| | IDPASu | — | total 0.06 μmol | total 0.3 (0.1 | ¹O₂ | U87MG | The present |
| | | | (0.038 mg) | Gy × 3 | | subcutaneous | disclosure |
| | | | | fractions) | | tumor (balb/c | |
| | | | | | | nude mice) | |
| | | | | | | (I.T.) | |
| | MRAP | — | total ~0.8 μmol | total 3.2 (0.8 | ¹O₂ | U87MG | |
| | | | (~2.96 mg) | Gy × 4 | | orthotopic | |
| | | | | fractions) | | tumor (balb/c | |
| | | | | | | nude mice) | |
| | | | | | | (I.V.) | |

Note:
I.V.: intravenous injection; I.T.: intratumoral injection; I.P.: Intraperitoneal injection, N.A: not available; Ref.: reference.

Here we report the development of organic luminophores that can efficiently undergo a radiodynamic process to generate singlet oxygen (¹O₂) and emit radio afterglow for precision cancer theranostics. In addition to their tunable near-infrared emission, long half-life and efficient ¹O₂ generation, the structure versatility of these radio-luminophores enables the construction of a smart activatable probe that only switches on its radio afterglow dynamic processes in the presence of the targeted cancer biomarker. In the above examples, we have described the synthesis and properties of these radio-luminophores, study the underlying mechanisms and potential advantages over light-induced processes as well as evaluate their potential in precision diagnosis and therapy for deep-seated brain tumours in preclinical settings.

In summary, we have developed a type of organic radioluminophore that outperforms inorganic nanophosphors to emit brighter, near-infrared radio afterglow and autonomously produce radiodynamic ¹O₂ without additives. The modular structure of these radio-luminophores facilitated the precise control of their physiological properties and construction of a smart molecular probe (MRAP) with unprecedented biomarker-activated radio afterglow dynamic function. Such a biomarker specificity of MRAP allowed the detection of a diminutive tumour with superb contrast and perform radiotherapy for deep-seated tumour with molecular precision at a low dosage. In addition, the modification of radioactive organic luminophores can potentially allow for image-guided surgery and monitoring cell therapy. Thus, our work not only reveals molecular guidelines to fill in the gap of organic radio afterglow agents but also highlights a new translational direction towards precision theranostics.

1. A compound of formula Ia or Ib: 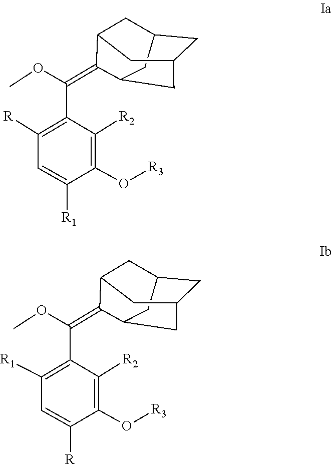
where:
R represents: 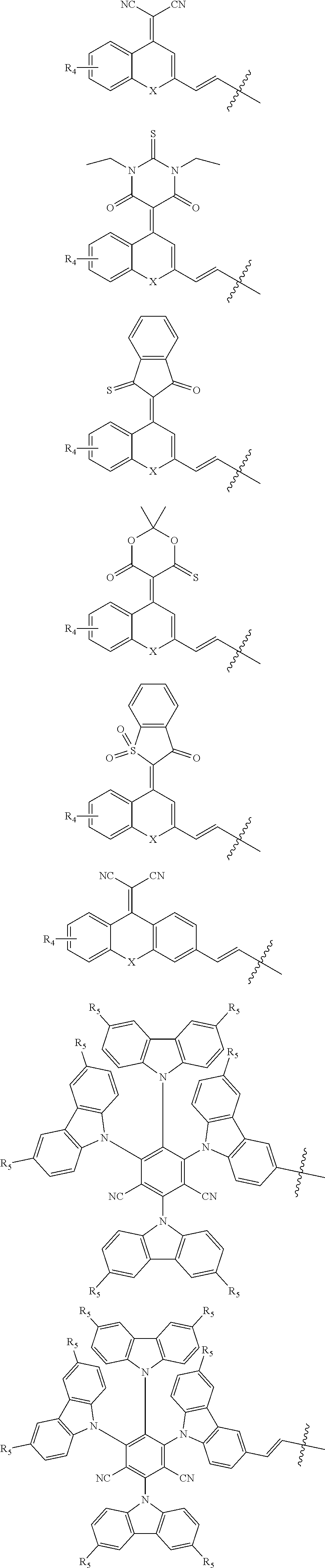
where the wiggly line represents the point of attachment to the rest of the molecule;
X represents NMe, NEt, CMe₂, SiMe₂, O, S or Se;
each R₄ is independently selected from:
H;
halo; 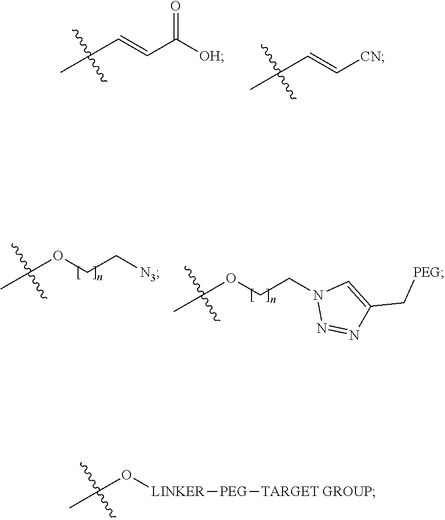
an aromatic or heteroaromatic group that is unsubstituted or substituted by CO₂H or OH,
where:
the wiggly line represents the point of attachment to the rest of the molecule;
PEG is PEG1K, PEG2K, or PEG5K;
n is 0 to 11;
LINKER represents: 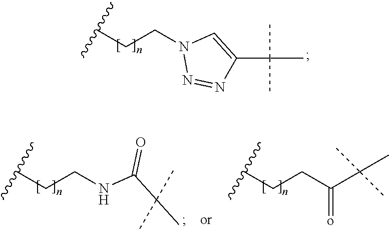
where n is 0 to 11 and the wiggly line represents the point of attachment to the oxygen atom and the dotted line represents the point of attachment to the TARGET GROUP;
TARGET GROUP represents: 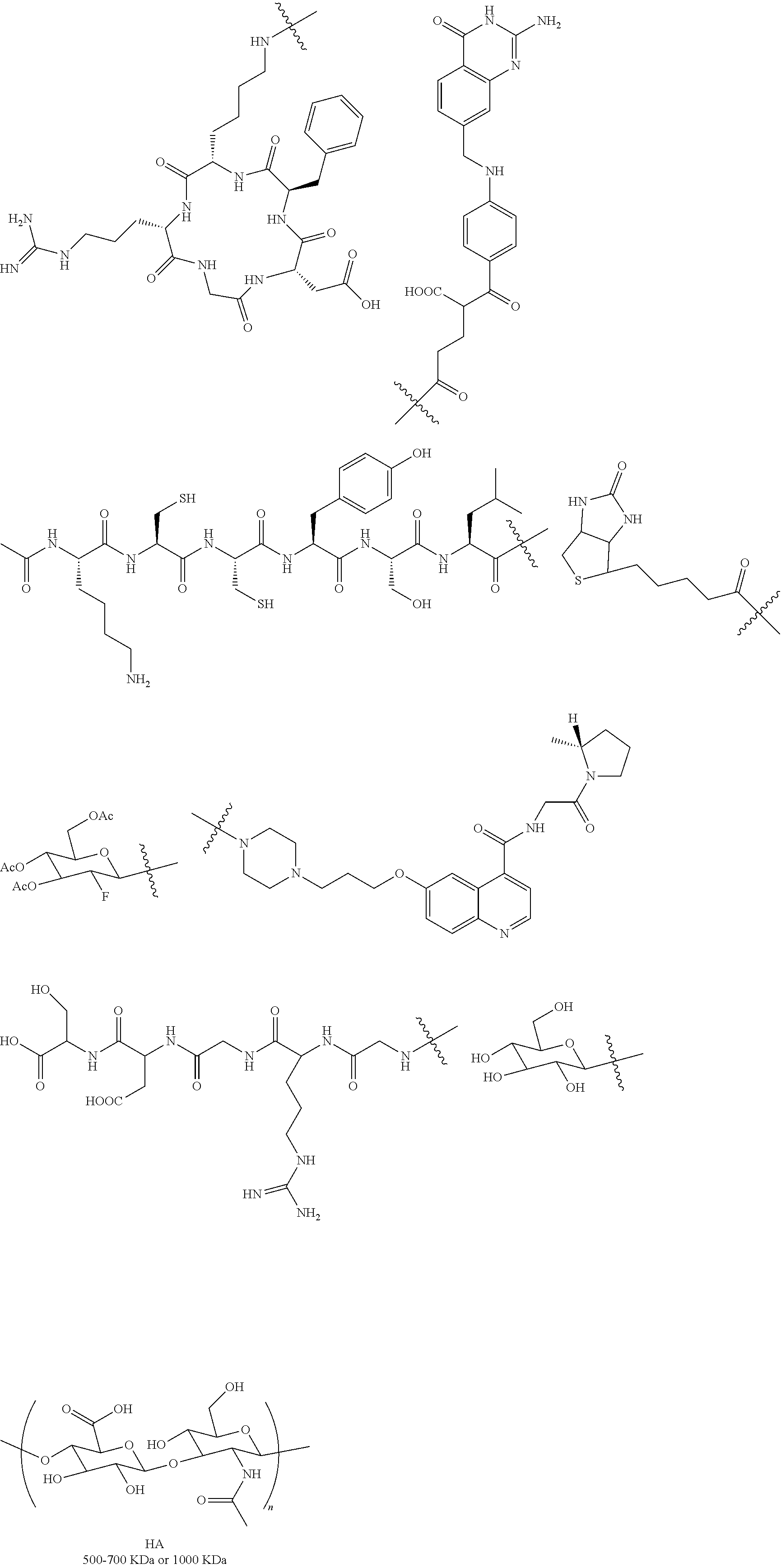
where the wiggly line represents the point of attachment to the rest of the molecule;
each R₅ independently represents H or halo;
each R₁ represents H, Br or I;
each R₂ represents H, CI, Br, or I;
R₃ either represents: 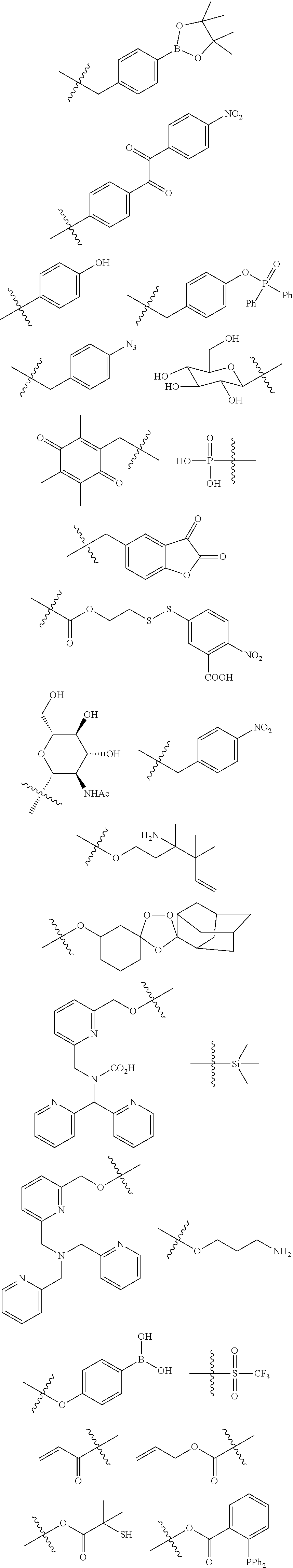
the wavy line represents the point of attachment to the rest of the molecule;
or R₃ represents -SELF IMMOLATIVE LINKER-PEPTIDE, where the self immolative linker is: 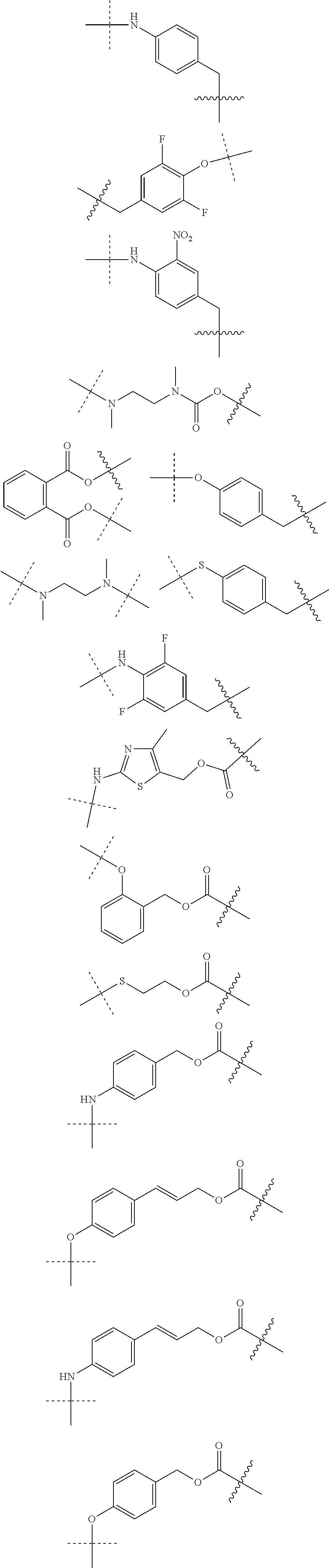
where the dotted line represents the point of attachment to the peptide and the wavy line represents the point of attachment to the rest of the molecule;
and the peptide is selected from: 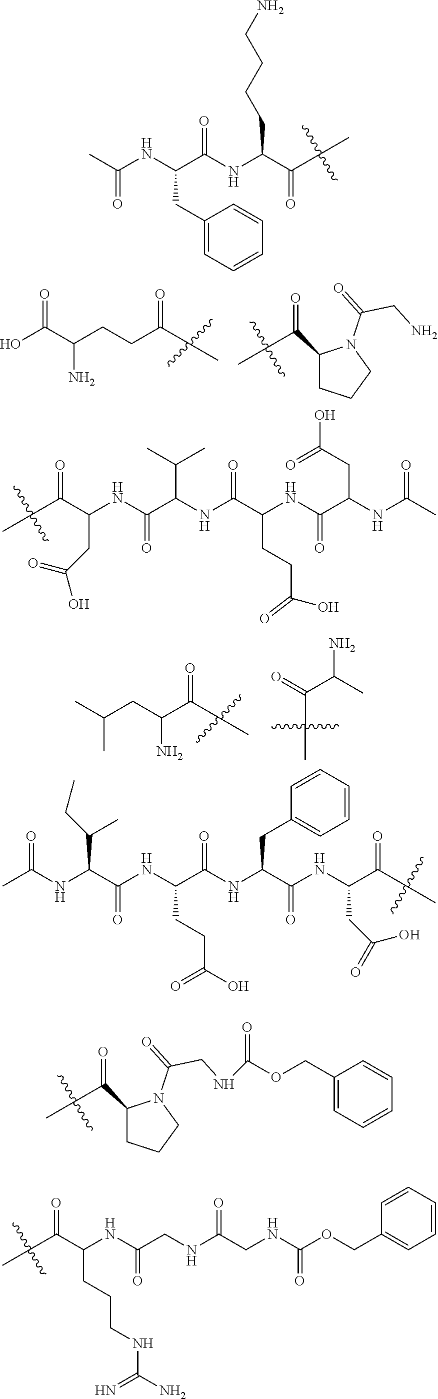
where the wavy line represents the point of attachment to the rest of the molecule,
or a pharmaceutically acceptable salt or solvate thereof.

2. The compound, or pharmaceutically acceptable salt or solvate thereof, according to claim 1, wherein the compound has formula Ia.

3. The compound, or pharmaceutically acceptable salt or solvate thereof, according to claim 1, wherein R represents: 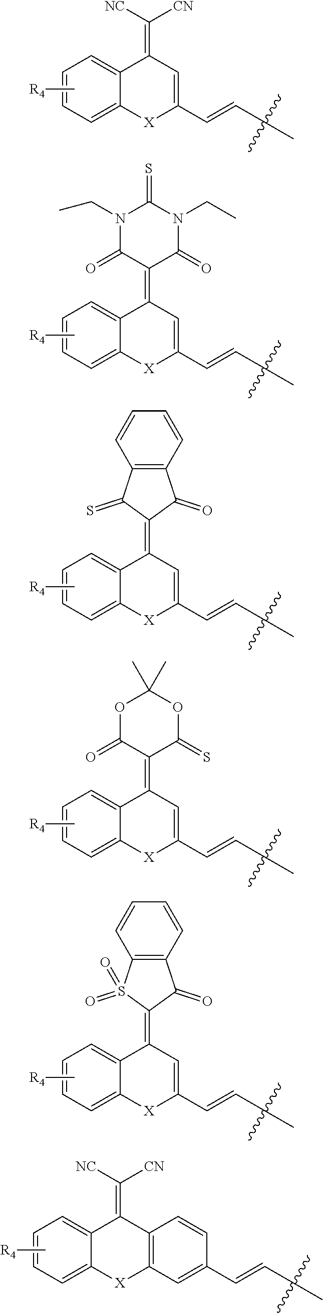
where the wiggly line represents the point of attachment to the rest of the molecule; and
X represents NEt, O, S or Se, such as O, S or Se.

4. The compound, or pharmaceutically acceptable salt or solvate thereof, according to claim 3, wherein R represents: 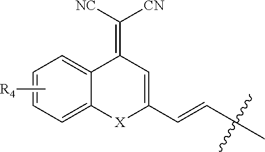
where the wiggly line represents the point of attachment to the rest of the molecule; and
X represents O, S or Se, such as S.

5. The compound, or pharmaceutically acceptable salt or solvate thereof, according to claim 1, wherein R₄ is independently selected from: 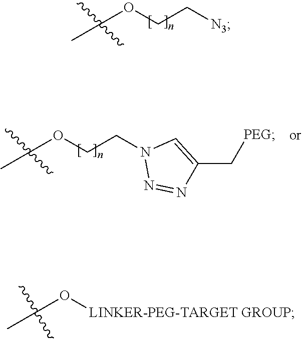
where:
the wiggly line represents the point of attachment to the rest of the molecule;
PEG is PEG1K, PEG2K, or PEG5K; and
n is 0 to 11.

6. The compound, or pharmaceutically acceptable salt or solvate thereof, according to claim 5, wherein R₄ is independently selected from: 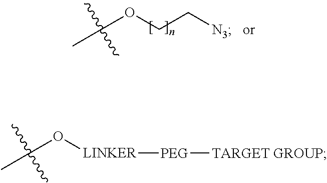
where:
the wiggly line represents the point of attachment to the rest of the molecule;
PEG is PEG1K, PEG2K, or PEG5K; and
n is 3 to 5, such as 4.

7. The compound, or pharmaceutically acceptable salt or solvate thereof, according to claim 1, wherein, when present, LINKER represents: 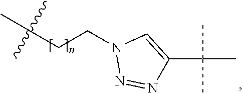
where n is 3 to 5, and the wiggly line represents the point of attachment to the oxygen atom and the dotted line represents the point of attachment to the TARGET GROUP.

8. The compound, or pharmaceutically acceptable salt or solvate thereof, according to claim 1, wherein, when present, TARGET GROUP represents: 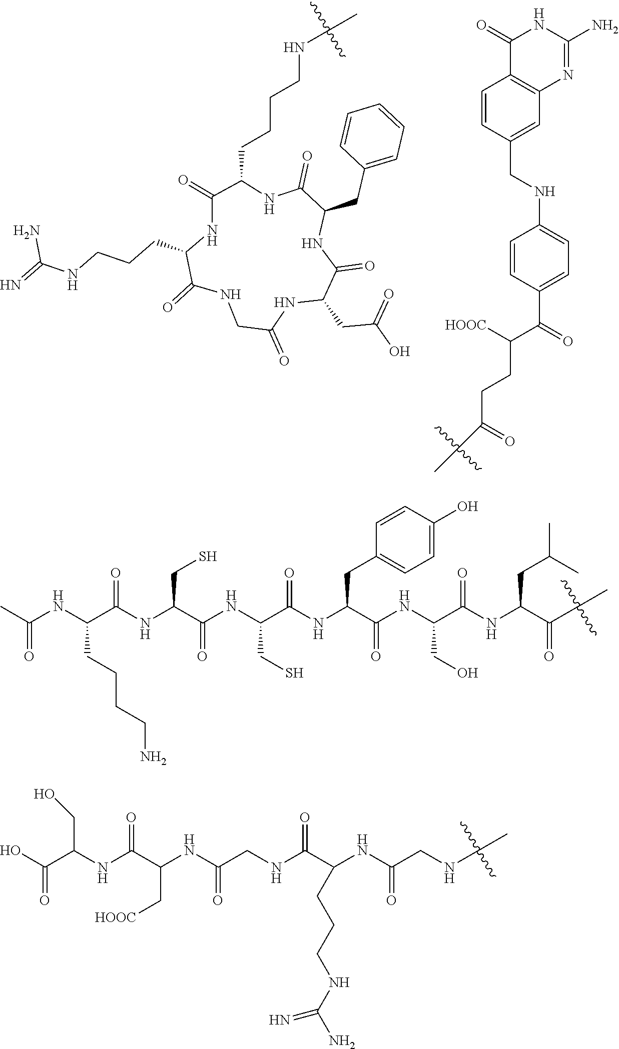 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O ; *C(=O)CCC(C(=O)O)C(=O)c1ccc(NCc2ccc3c(=O)[nH]c(N)nc3c2)cc1 ; *C(=O)[C@H](CC(C)C)NC(=O)[C@H](CO)NC(=O)[C@H](Cc1ccc(O)cc1)NC(=O)[C@H](CS)NC(=O)[C@H](CS)NC(=O)[C@H](CCCCN)NC(C)=O ; *NCC(=O)NC(CCCNC(=N)N)C(=O)NCC(=O)NC(CC(=O)O)C(=O)NC(CO)C(=O)O]
where the wiggly line represents the point of attachment to the rest of the molecule.

9. The compound, or pharmaceutically acceptable salt or solvate thereof, according to claim 8, wherein, when present, TARGET GROUP represents: 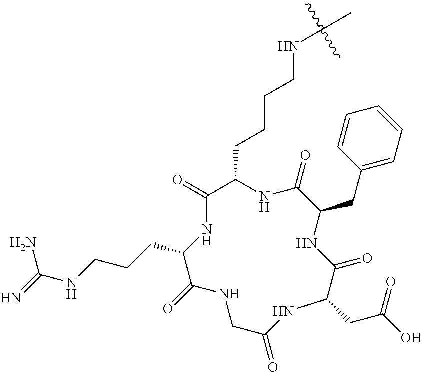 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O]
where the wiggly line represents the point of attachment to the rest of the molecule.

10. The compound, or pharmaceutically acceptable salt or solvate thereof, according to claim 1, wherein R₁ represents H or I.

11. The compound, or pharmaceutically acceptable salt or solvate thereof, according to claim 1, wherein R₂ represents H.

12. The compound, or pharmaceutically acceptable salt or solvate thereof, according to claim 1, wherein R₃ either represents: 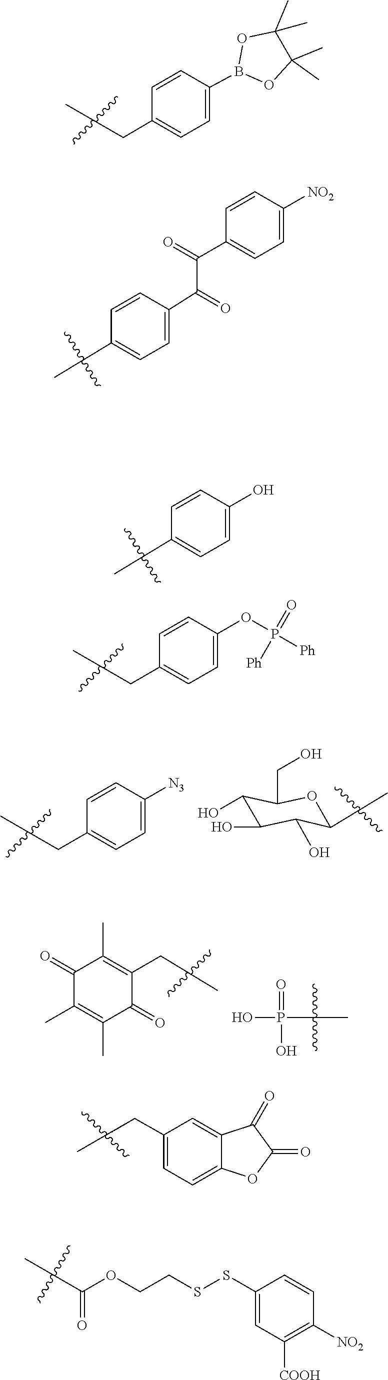 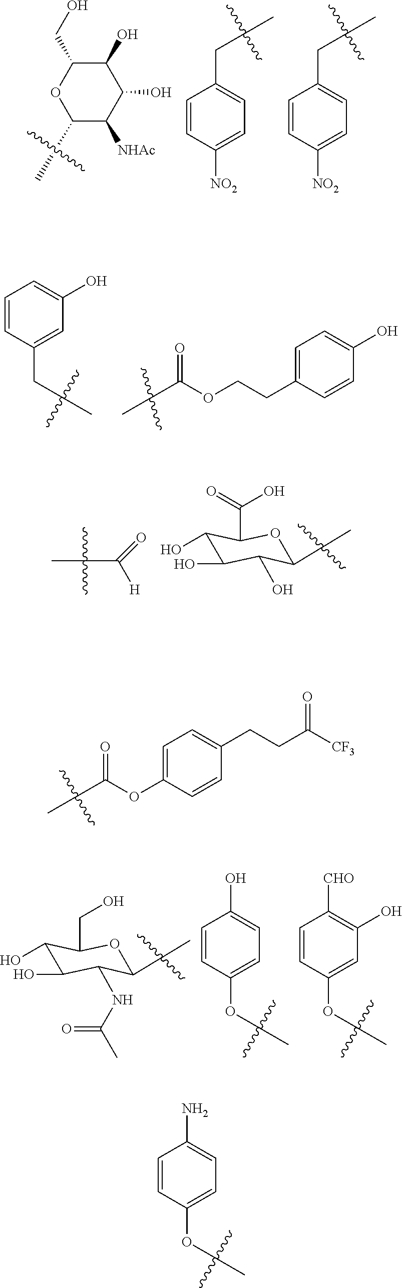
the wavy line represents the point of attachment to the rest of the molecule;
or R₃ represents -SELF IMMOLATIVE LINKER-PEPTIDE, where the self immolative linker is: 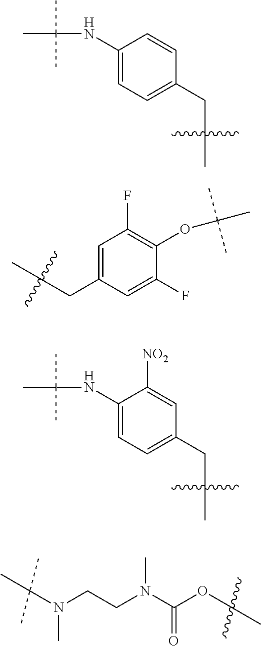
where the dotted line represents the point of attachment to the peptide and the wavy line represents the point of attachment to the rest of the molecule.

13. The compound, or pharmaceutically acceptable salt or solvate thereof, according to claim 1, wherein R₃ represents -SELF IMMOLATIVE LINKER-PEPTIDE, where the self immolative linker is: 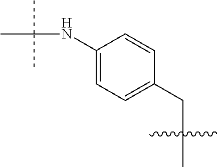
where the dotted line represents the point of attachment to the peptide and the wavy line represents the point of attachment to the rest of the molecule.

14. The compound, or pharmaceutically acceptable salt or solvate thereof, according to claim 1, wherein when R₃ represents 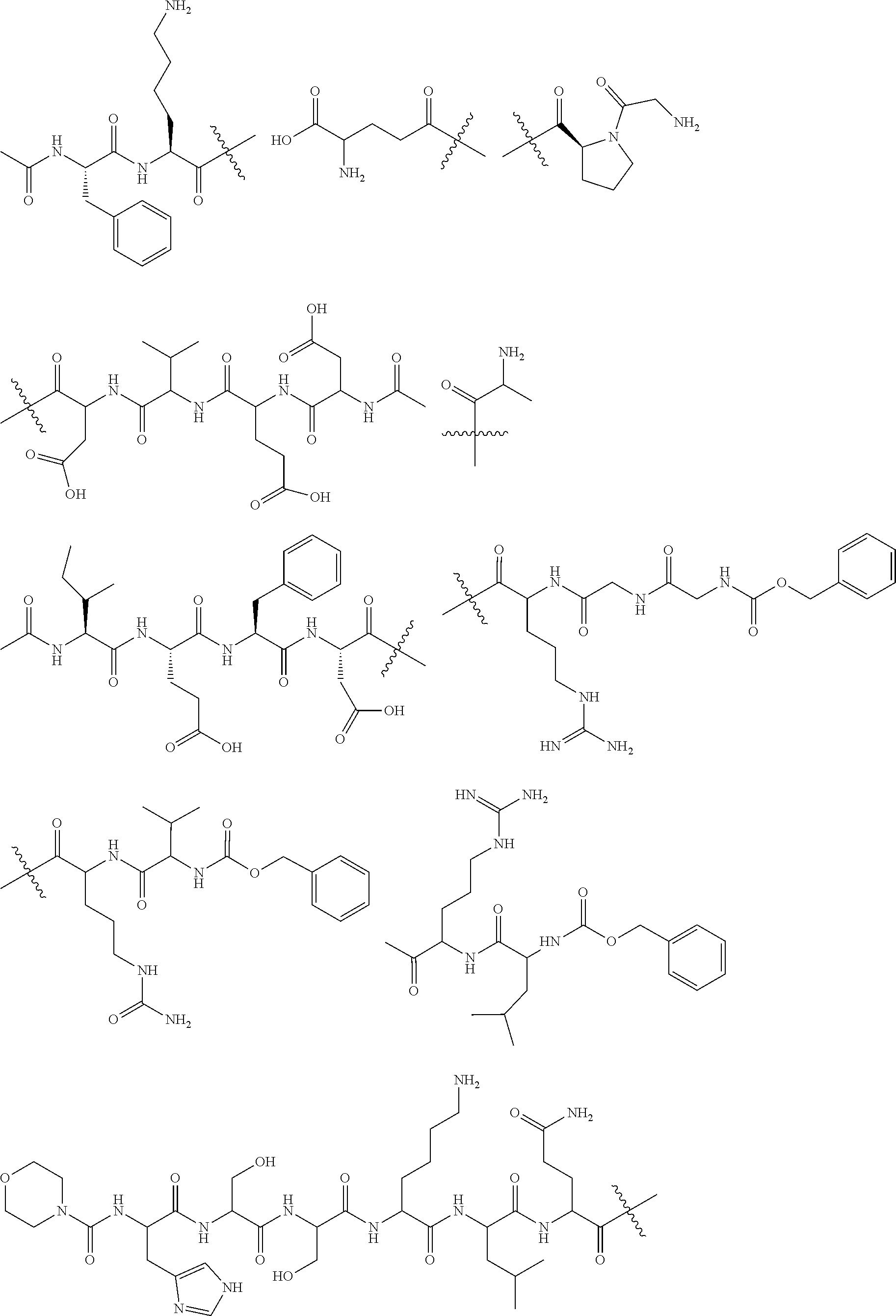
where the wavy line represents the point of attachment to the rest of the molecule.

15. The compound, or pharmaceutically acceptable salt or solvate thereof, according to claim 14, wherein when R₃ represents 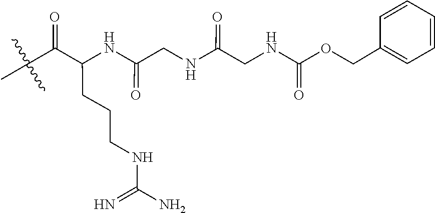 [SMILES: *C(=O)C(CCCNC(=N)N)NC(=O)CNC(=O)CNC(=O)OCc1ccccc1]
where the wavy line represents the point of attachment to the rest of the molecule.

16. The compound, or pharmaceutically acceptable salt or solvate thereof, according to claim 1, wherein:
R₁ is I;
R₂ is H;
R is: 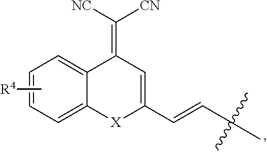
where the wavy line represents the point of attachment to the rest of the molecule;
R₄ is 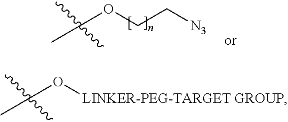
where LINKER is 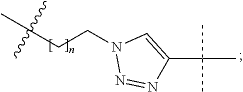
n is 4;
PEG is PEG2K;
Target Group is 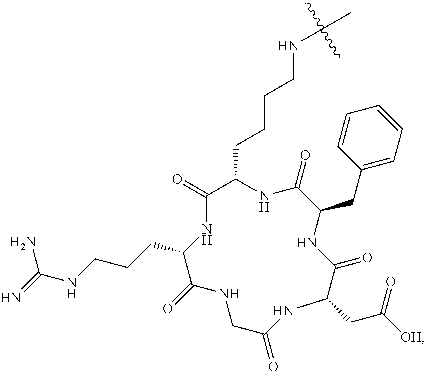 [SMILES: *NCCCC[C@@H]1NC(=O)[C@@H](Cc2ccccc2)NC(=O)[C@H](CC(=O)O)NC(=O)CNC(=O)[C@H](CCCNC(=N)N)NC1=O]
where the wiggly line represents the point of attachment to the rest of the molecule;
R₃ is -SELF IMMOLATIVE LINKER-PEPTIDE, where the self immolative linker is 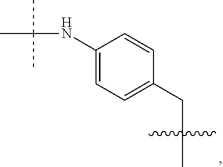
where the dotted line represents the point of attachment to the peptide and the wavy line represents the point of attachment to the rest of the molecule; and
the peptide is 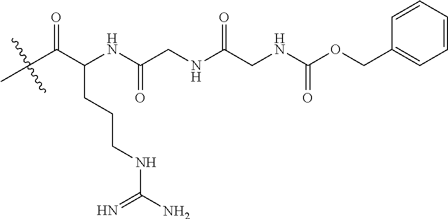 [SMILES: *C(=O)C(CCCNC(=N)N)NC(=O)CNC(=O)CNC(=O)OCc1ccccc1]
where the wavy line represents the point of attachment to the rest of the molecule.

17. A method for detection of cancer in vivo, the method comprising the following steps:
(ai) administering a compound of formula Ia or Ib, or pharmaceutically acceptable salt or solvate thereof, according to claim 1 to a subject; and
(aii) detecting any chemiluminescence, wherein the presence of cancer in vivo is indicated by chemiluminescence.

18. A method of treating cancer, the method comprising the following steps:
(bi) administering a compound of formula Ia or Ib, or pharmaceutically acceptable salt or solvate thereof, according to claim 1 to a subject; and
(bii) subjecting the subject to X-ray irradiation.

19. The method according to claim 18, wherein the compound of formula Ia or formula Ib, or salt or solvate thereof, is a compound in which R₄ is [chemical structure]
